(19) United States
(12) Patent Application Publication
(10) Pub. No.: US 2010/0286191 A1
(43) Pub. Date: Nov. 11, 2010

(54) 2-Arylthiazole Derivatives as CXCR3 Receptor Modulators

(76) Inventors: Rachel G. Samuel, Ridgefield, NJ (US); Conrad Santini, Stirling, NJ (US)

Correspondence Address: MERCK, P O BOX 2000, RAHWAY, NJ, 07065-0907, US

(21) Appl. No.: 12/086,361

(22) PCT Filed: Dec. 8, 2006
(86) PCT No.: PCT/US06/47065

§ 371 Date: Jun. 11, 2008

Related U.S. Application Data
(60) Provisional application No. 60/749,419, filed on Dec. 12, 2005.

(51) Int. Cl. A61K 31/454; C07D 471/04; A61K 31/444; C07D 417/14; A61P 17/06; A61P 25/00; A61P 29/00; A61P 37/06

(52) U.S. Cl. 514/303; 546/118; 546/209; 514/326; 546/199; 514/322; 546/193; 514/318

(57) ABSTRACT

The invention encompasses compounds of Formula I or pharmaceutically acceptable salts thereof, which are modulators of the CXCR3 chemokine receptor function useful for the treatment or prevention of pathogenic inflammatory processes, autoimmune diseases or graft rejection processes. Methods of use and pharmaceutical compositions are also encompassed.

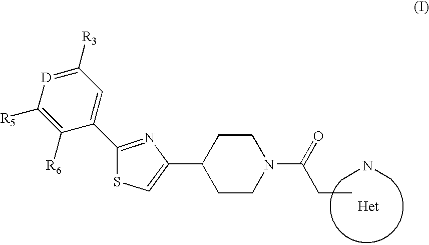

### BACKGROUND OF THE INVENTION

The chemokines are a family of small (70-120 amino acids), pro-inflammatory cytokines, with potent chemotactic activities. As their name implies, one function of chemokines, which are released by a wide variety of cells at sites of inflammation, is to attract leukocytes, including monocytes, macrophages, T lymphocytes, eosinophils, basophils and neutrophils and to promote their migration through endothelial layers. (reviewed in Schall, Cytokine, 3, 165-183 (1991) and Murphy, Rev. Immun., 12, 593-633 (1994)). In addition to their well characterized role in leukocyte trafficking, it is now also appreciated that that chemokines play a role in a number of other biological processes including cellular proliferation, hematopoiesis, angiogenesis, tumor metastasis and host defense.

These polypeptides were originally defined as having four conserved aminoterminal cysteines, and divided into two major and two minor subfamilies based on the spacing arrangement of the first cysteine pair. The two major subfamilies consist of the CXC (or α) and CC (or β) chemokines. In the CXC-chemokine family, which includes CXCL1 (MGSA or GROα), CXCL7 (NAP-2), CXCL8 (interleukin-8 or IL-8), CXCL9 (MIG), CXCL10 (IP-10) and CXCL11 (I-TAC), these two cysteines are separated by a single amino acid, while in the CC-chemokine family, which includes CCL5 (RANTES), CCL2 (monocyte chemotactic protein-1 or MCP-1), CCL8 (MCP-2), CCL7 (MCP-3), CCL3 (MIP-1α), CCL4 (MIP-1β) and CCL11 (eotaxin), these two residues are adjacent.

Some CXC-chemokines, such as CXCL1, CXCL7 and CXCL-8 are chemotactic primarily for neutrophils while another subset of CXC chemokines, including CXCL9, CXCL10 and CXCL11, are chemotactic primarily for T-lymphocytes. In comparision, the CC_chemokines, such as CCL5, CCL3, CCL4, CCL2, CCL8, CCL7 and CCL11, are more broad in their action and are chemotactic for macrophages, monocytes, T-lymphocytes, eosinophils and basophils (Deng, et al., Nature, 381, 661-666 (1996), Murphy et al. Pharmacol Revw. 52(1) 145-176, (2000).).

The chemokines bind to specific G-protein coupled receptors (GPCRs) present on leukocytes and other cells. (reviewed in Horuk, Trends Pharm. Sci., 15, 159-165 (1994), Murphy et al. Pharmacol Revw. 52(1) 145-176, (2000).) Upon interaction with their cognate ligands, chemokine receptors transduce an intracellular signal though their associated heterotrimeric G proteins, resulting in a rapid cellular responses, including an increase in intracellular calcium concentration. These chemokine receptors form a sub-family of GPCRs, which, at present, consists of a number of well characterized members with known ligands as well as a number of orphans. Unlike receptors for promiscuous classical chemoattractants such as C5a, fMLP, PAF, and LTB4, chemokine receptors are more selectively expressed on subsets of leukocytes. Thus, generation of specific chemokines provides a mechanism for recruitment of particular leukocyte subsets. The restricted expression and defined function of the chemokine receptors has focused attention on intervention in the chemokine signaling pathways as a method for highly selective intervention in pathological immunological and inflammatory processes.

Chemokine receptors, such as CCR1, CCR2A, CCR2B, CCR3, CCR4, CCR5, CXCR3, CXCR4, have been implicated as important mediators of inflammatory diseases and immunoregulatory disorders, including asthma, allergic rhinitis and and atherosclerosis. They are also purported to play a role in the pathogenesis of autoimmune disorders such as rheumatoid arthritis, psoriasis, multiple sclerosis. An extensive review of the role of chemokines in disease is provided by in Seminars in Immunology., 15(1), 1-55 (2003).

A subset of chemokines are potent chemoattractants for lymphocytes. For example CXCR3 (CD183) is expressed in activated T lymphocytes, some B lymphocytes and NK cells. Expression and receptor responsiveness are both increased by activation of the T lymphocytes. The potent inflammatory cytokines CXCL10 and CXCL11 are chemoattractant for T lymphocytes and tumor infiltrating lymphocytes. The relatively restricted expression of the CXCR3 expression on these pro-inflammatory cell types mark CXCR3 as a very promising target for selective intervention in the inflammatory process. A connection with disease processes, particularly Th-1 mediated processes, is indicated by the presence of the CXCR3 on most activated T lymphocytes within inflamed joint synovium in rheumatoid arthritis as well as within inflamed tissue present in other inflammatory disorders including ulcerative colitis, Graves' disease, MS and rejecting graft tissues. (Qin, J. Clin. Invest., 101(4), 746-754 (1998), Garcia-Lopez, Lab. Investig. 81(3), 409-418 (2001), Balashov, PNAS, 96, 6873-6878 (1999), DeVries, Seminars in Immunology, 15(1), 33-48 (2003)). A similar but somewhat less pronounced association is shown with the CCR5 receptor and its ligand CCL5

Accordingly, agents which inhibit or modulate the function of chemokine receptors such as the CXCR3 receptor would be useful in treating or preventing such disorders and diseases. Data from animal models of inflammation further supports the hypothesis regarding the effectiveness of chemokine blockade, specifically CXCR3 inhibition, in diseases with clear T-lymphocyte mediated tissue damage such as transplant rejection, graft versus host disease, multiple sclerosis, optic neuritis and rheumatoid or psoriatic arthritis. Many other diseases are characterized by T lymphocyte infiltrates, and by inference are therefore also good candidates for interventions which prevent the migration of T lymphocytes. These diseases include psoriasis and other chronic inflammatory diseases of the skin such as atopic dermatitis, lichen planus and bullous pemphigoid, inflammatory bowel diseases such as ulcerative colitis and Crohn's disease and autoimmune diseases such as systemic and cutaneous lupus erythematosus, Behcet's disease, type I diabetes or Graves' disease.

Many inflammatory lung diseases such as chronic obstructive pulmonary disease, hypersensitivity pneumonitis, chronic eosinophilic pneumonia, pulmonary sarcoidosis, bronchiolitis obliterans syndrome, asthma, kidney diseases such as glomerulonephritis, pathogenesis of chronic HCV infection and atherosclerosis show a dependence on T lymphocytes and are promising targets for agents which modulate the function of chemokine receptors such as the CXCR3 receptor.

The expression of CXCR3 in some B cell tumors indicates that intervention in CXCR3 function could have beneficial effects in these cancers, particularly in suppressing metastasis.

Several methods are under investigation for modulation of chemokine receptor function. These include antibodies binding to and neutralizing the chemokine ligands, antibodies binding to and modulating the function of the chemokine receptors and small molecules which bind to and inhibit function of the chemokine receptor. The ideal method for intervention in CXCR3 mediated chemotaxis is the binding of orally bioavailable small molecules which prevent the function of the receptor. Molecules with affinity for the CXCR3 chemokine receptor and ability to modulate the function of the receptor are described here.

### SUMMARY OF THE INVENTION

The invention encompasses compounds of Formula I

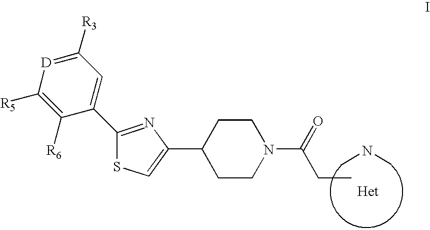

or pharmaceutically acceptable salts thereof, which are modulators of the CXCR3 chemokine receptor function useful for the treatment or prevention of pathogenic inflammatory processes, autoimmune diseases or graft rejection processes. Methods of use and pharmaceutical compositions are also encompassed.

### DETAILED DESCRIPTION OF THE INVENTION

The invention encompasses a genus of compounds of Formula I

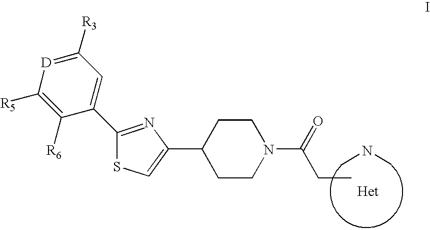

or a pharmaceutically acceptable salt thereof, wherein:
D is CR₄ or N;
R₃ is selected from the group consisting of: H, halo, C₁₋₄alkyl, —CF₃, —OCF₃ and —S(O)ₙCF, wherein n is 0 or 2;
R₄ is selected from the group consisting of: H, halo, —OH, C₁₋₄alkyl, —OCH₃, —OCH₂CF₃ and —CF₃;
or R₃ and R₄ may be joined together with the carbon atoms to which they are attached to form a five- or six-membered monocyclic ring, said rings containing oxygen or tetra-substituted with methyl groups as follows:

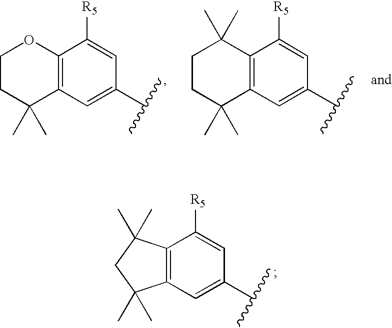

R₅ is selected from the group consisting of: —H, halo, C₁₋₄alkyl, C₃₋₆cycloalkyl, CF₃, —CF₂CH₃, —OCF₃ and —SCF₃;
R₆ is selected from the group consisting of —H and —OCH₃,
or R₅ and R₆ may be joined together with the carbon atoms to which they are attached to form a monocyclic 5-membered ring, said ring di-substituted with methyl as follows:

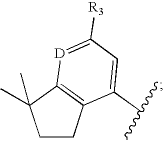

and

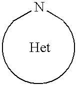

is a 5 membered non-aromatic or aromatic ring or a 9 membered fused bicyclic partially aromatic or aromatic ring, each ring containing at least 1 nitrogen atom and optionally up to 3 additional heterotaoms selected from S, O and N, said rings optionally substituted with 1 to 3 substituents independently selected from the group consisting of: oxo, hydroxy, carboxy, —CF₃, halo, —S(O)ₚ—CH₃, phenyl, C₁₋₃alkoxy and C₁₋₃alkyl, said C₁₋₃alkyl optionally substituted with carboxy or hydroxy; and
p is 0, 1 or 2.

Within this genus, the invention encompasses a sub-genus of compounds of Formula I wherein:

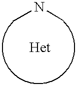

is selected from the group consisting of:

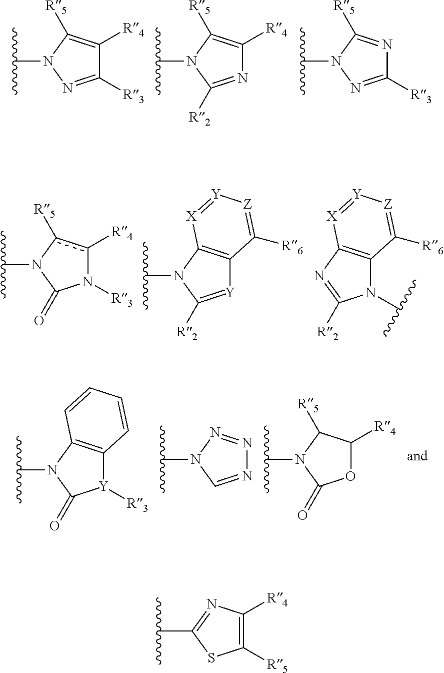

wherein X, Y and Z are independently C or N,
R″₂, R″₃, R″₄ and R″₅ are independently selected from the group consisting of: —H, carboxy, —CF₃, halo, methylthio, methylsulfonyl, phenyl, C₁₋₃alkoxy and C₁₋₃alkyl, said C₁₋₃alkyl optionally substituted with carboxy or hydroxy,
R″₆ is H or OH, and
------ is an optional double bond.

Also within this genus, the invention encompasses a sub-genus of compounds of Formula I wherein D is N.

Also within this genus, the invention encompasses a sub-genus of compounds of Formula I wherein D is CR₄.

Within this sub-genus, the invention encompasses a class of compounds of Formula I wherein:
R₃ and R₄ are joined together with the carbon atoms to which they are attached to form a six-membered monocyclic ring as follows:

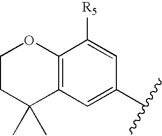

Also within this sub-genus, the invention encompasses a class of compounds of Formula I wherein:
R₃ is selected from the group consisting of: H, halo, C₁₋₄alkyl and —CF₃;
R₄ is selected from the group consisting of: H, halo, C₁₋₄alkyl, —OCH₃ and —CF₃;
R₅ is selected from the group consisting of: H, halo, C₁₋₄alkyl, CF₃ and —SCF₃; and
R₆ is H.

Within this class, the invention encompasses a sub-class of compounds of Formula I wherein:
R₃ is selected from the group consisting of: H, Cl, Br, tert-butyl and —CF₃;
R₄ is selected from the group consisting of H, Cl, Br, tert-butyl, —OCH₃ and —CF₃;
R₅ is selected from the group consisting of: H, Cl, Br, tert-butyl, CF₃ and —SCF₃; and
R₆ is H.

Another embodiment of the invention encompasses a sub-genus of compounds of Formula I within the above-described genus wherein R₆ is —H.

Another embodiment of the invention encompasses a sub-genus of compounds of Formula I within the above-described genus wherein:
D is CR₄;
R₃ is selected from the group consisting of: H, Cl, Br, tert-butyl and —CF₃;
R₄ is selected from the group consisting of H, Cl, Br, tert-butyl, —OCH₃ and —CF₃;
or R₃ and R₄ may be joined together with the carbon atoms to which they are attached to form a six-membered monocyclic ring as follows:

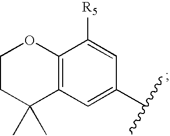

R₅ is selected from the group consisting of H, Cl, Br, tert-butyl, CF₃ and —SCF₃; and
R₆ is —H.

Within this sub-genus, the invention encompasses a class of compounds of Formula I wherein:
(1) R₃ is tert-butyl, R₄ is H and R₅ is tert-butyl;
(2) R₃ is tert-butyl, R₄ is —OCH₃ and R₅ is tert-butyl;
(3) R₃ is —CF₃, R₄ is —H and R₅ is —CF₃;
(4) R₃ is H, R₄ is —OCH₃ and R₅ is H;
(5) R₃ is H, R₄ is tert-butyl and R₅ is H;
(6) R₃ is H, R₄ is Cl and R₅ is H;
(7) R₃ is H, R₄ is Br and R₅ is H;
(8) R₃ is H, R₄ is —CF₃ and R₅ is H;
(9) R₃ is H, R₄ is H and R₅ is Cl;
(10) R₃ is H, R₄ is H and R₅ is Cl;
(11) R₃ is H, R₄ is H and R₅ is —CF₃; or
(12) R₃ is Cl, R₄ is H and R₅ is Cl.

Also within this sub-genus, the invention encompasses a class of compounds of Formula I wherein

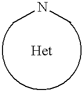

is selected from the group consisting of:

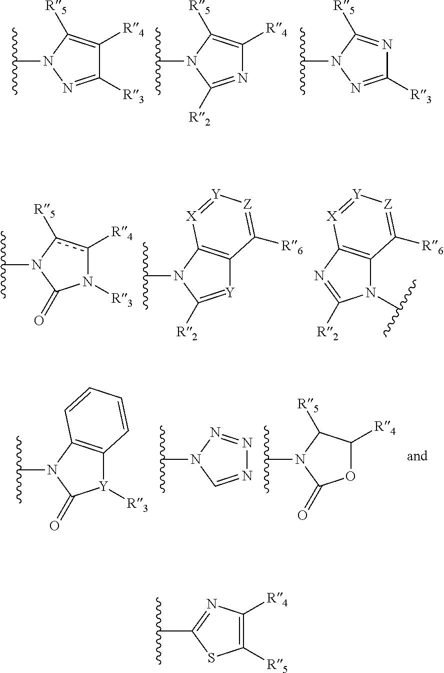

wherein X, Y and Z are independently C or N,
R″₂, R″₃, R″₄ and R″₅ are independently selected from the group consisting of: —H, arboxy, —CF₃, halo, methylthio, methylsulfonyl, phenyl, C₁₋₃alkoxy and C₁₋₃alkyl, said C₁₋₃alkyl optionally substituted with carboxy or hydroxy,
R″₆ is H or OH, and
------ is an optional double bond.

Within this class, the invention encompasses a sub-class of compounds of Formula I wherein:

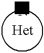

is

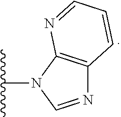

Also within this sub-genus, the invention encompasses a class of compounds of Formula I wherein:
D is CR₄;
R₃ is tert-butyl;
R₄ is H or —OCH₃;
R₅ is tert-butyl; and
R₆ is H.

In another embodiment, the invention encompasses a compound selected from the following group:
(1) 3-(2-{4-[2-(3,5-di-tert-butyl-4-methoxyphenyl)-1,3-thiazol-4-yl]piperidin-1-yl}-2-oxoethyl)-3H-imidazo[4,5-b]pyridine;
(2) 4-[2-(3,5-di-tert-butyl-4-methoxyphenyl)-1,3-thiazol-4-yl]-1-[(2,5-dimethyl-1H-imidazol-1-yl)acetyl]piperidine;
(3) [1-(2-{4-[2-(3,5-di-tert-butyl-4-methoxyphenyl)-1,3-thiazol-4-yl]piperidin-1-yl}-2-oxoethyl)-2-methyl-1H-imidazol-5-yl]methanol;
(4) 1-(2-{4-[2-(3,5-di-tert-butyl-4-methoxyphenyl)-1,3-thiazol-4-yl]piperidin-1-yl}-2-oxoethyl)-3-methyl-1,3-dihydro-2H-imidazol-2-one;
(5) 3-(2-{4-[2-(3,5-di-tert-butyl-4-methoxyphenyl)-1,3-thiazol-4-yl]piperidin-1-yl}-2-oxoethyl)-1,4-dimethyl-1,3-dihydro-2H-imidazol-2-one;
(6) [1-(2-{4-[2-(3,5-di-tert-butyl-4-methoxyphenyl)-1,3-thiazol-4-yl]piperidin-1-yl}-2-oxoethyl)-4-methyl-1H-pyrazol-3-yl]acetate bis(hydrotrifluoroacetate);
(7) 4-[2-(3,5-di-tert-butyl-4-methoxyphenyl)-1,3-thiazol-4-yl]-1-{[4-methyl-2-(methylsulfonyl)-1H-imidazol-1-yl]acetyl}piperidine;
(8) 1-(2-{4-[2-(3,5-di-tert-butyl-4-methoxyphenyl)-1,3-thiazol-4-yl]piperidin-1-yl}-2-oxoethyl)-1,3-dihydro-2H-imidazol-2-one;
(9) Potassium_{—}[3-(2-{4-[2-(3,5-di-tert-butyl-4-methoxyphenyl)-1,3-thiazol-4-yl]piperidin-1-yl}-2-oxoethyl)-2-oxo-2,3-dihydro-1H-imidazol-1-yl]acetate;
(10) 1-(2-{4-[2-(3,5-di-tert-butyl-4-methoxyphenyl)-1,3-thiazol-4-yl]piperidin-1-yl}-2-oxoethyl)-3-phenylimidazolidin-2-one;
(11) 3-{2-[4-(2-{3-tert-butyl-5-[(trifluoromethyl)thio]phenyl}-1,3-thiazol-4-yl)piperidin-1-yl]-2-oxoethyl}-3H-imidazo[4,5-b]pyridine;
(12) 4-[2-(3,5-di-tert-butyl-4-methoxyphenyl)-1,3-thiazol-4-yl]-1H-[(4-methoxy-1-imidazol-1-yl)acetyl]piperidine;
(13) 4-[2-(8-tert-butyl-4,4-dimethyl-3,4-dihydro-2H-chromen-6-yl)-1,3-thiazol-4-yl]-1-[(2,4-dimethyl-1H-imidazol-1-yl)acetyl]piperidine;
(14) 1-[(4-chloro-2,5-dimethyl-1H-imidazol-1-yl)acetyl]-4-[2-(3,5-di-tert-butyl-4-methoxyphenyl)-1,3-thiazol-4-yl]piperidine;
(15) 3-(2-{4-[2-(3,5-di-tert-butylphenyl)-1,3-thiazol-4-yl]piperidin-1-yl}-2-oxoethyl)-3H-imidazo[4,5-c]pyridine;
(16) 1-(2-{4-[2-(3,5-di-tert-butylphenyl)-1,3-thiazol-4-yl]piperidin-1-yl}-2-oxoethyl)-1H-imidazo[4,5-c]pyridine;
(17) 3-(2-{4-[2-(3,5-di-tert-butylphenyl)-1,3-thiazol-4-yl]piperidin-1-yl}-2-oxoethyl)-3H-imidazo[4,5-b]pyridinel;
(18) 1-(2-{4-[2-(3,5-di-tert-butylphenyl)-1,3-thiazol-4-yl]piperidin-1-yl}-2-oxoethyl)-1H-imidazo[4,5-b]pyridine;
(19) 3-(2-{4-[2-(3,5-di-tert-butyl-4-methoxyphenyl)-1,3-thiazol-4-yl]piperidin-1-yl}-2-oxoethyl)-1-ethyl-4-methyl-1,3-dihydro-2H-imidazol-2-one;
(20) 1-(2-{4-[2-(3,5-di-tert-butylphenyl)-1,3-thiazol-4-yl]piperidin-1-yl}-2-oxoethyl)-3-ethyl-4-methyl-1,3-dihydro-2H-imidazol-2-one;
(21) 1-(2-{4-[2-(3,5-di-tert-butyl-4-methoxyphenyl)-1,3-thiazol-4-yl]piperidin-1-yl}-2-oxoethyl)-3-methyl-1,3-dihydro-2H-benzimidazol-2-one;
(22) 4-[2-(3,5-di-tert-butyl-4-methoxyphenyl)-1,3-thiazol-4-yl]-1-[(2-ethyl-4-methyl-1H-imidazol-1-yl)acetyl]piperidine;
(23) 2,6-di-tert-butyl-4-{4-[1-(1H-imidazo[4,5-c]pyridin-1-ylacetyl)piperidin-4-yl]-1,3-thiazol-2-yl}phenol;
(24) 2,6-di-tert-butyl-4-{4-[1-(3H-imidazo[4,5-c]pyridin-3-ylacetyl)piperidin-4-yl]-1,3-thiazol-2-yl}phenol;
(25) 2,6-di-tert-butyl-4-{4-[1-(3H-imidazo[4,5-b]pyridin-3-ylacetyl)piperidin-4-yl]-1,3-thiazol-2-yl}phenol;
(26) 2,6-di-tert-butyl-4-(4-{1-[(3,5-dimethyl-1H-1,2,4-triazol-1-yl)acetyl]piperidin-4-yl}-1,3-thiazol-2-yl)phenol;
(27) [1-(2-{4-[2-(3,5-di-tert-butyl-4-methoxyphenyl)-1,3-thiazol-4-yl]piperidin-1-yl}-2-oxoethyl)-4-methyl-1H-imidazol-5-yl]methanol;
(28) 4-[2-(3,5-di-tert-butyl-4-methoxyphenyl)-1,3-thiazol-4-yl]-1-{2-(methylthio)-1H-imidazol-1-yl]acetyl}piperidine;
(29) 4-[2-(3,5-di-tert-butyl-4-methoxyphenyl)-1,3-thiazol-4-yl]-1-{2-(methylsulfonyl)-1H-imidazol-1-yl]acetyl}piperidine;
(30) [1-(2-{4-[2-(3,5-di-tert-butyl-4-methoxyphenyl)-1,3-thiazol-4-yl]piperidin-1-yl}-2-oxoethyl) 5-methyl-1H-imidazol-4-yl]acetic dihydrochloride;
(31) [1-(2-{4-[2-(3,5-di-tert-butyl-4-methoxyphenyl)-1,3-thiazol-4-yl]piperidin-1-yl}-2-oxoethyl)-4-methyl-1H-imidazol-5-yl]acetic dihydrochloride;
(32) 4-[2-(3,5-di-tert-butyl-4-methoxyphenyl)-1,3-thiazol-4-yl]-1-[(3,5-dimethyl-1H-1,2,4-triazol-1-yl)acetyl]piperidine;
(33) 4-[2-(3,5-di-tert-butylphenyl)-1,3-thiazol-4-yl]-1-[(3,5-dimethyl-1H-1,2,4-triazol-1-yl)acetyl]piperidine;
(34) 4-[2-(3,5-di-tert-butyl-4-methoxyphenyl)-1,3-thiazol-4-yl]-1-[(4-methyl-1-imidazol-1-yl)acetyl]piperidine;
(35) 4-[2-(3,5-di-tert-butyl-4-methoxyphenyl)-1,3-thiazol-4-yl]-1-[(5-methyl-1-imidazol-1-yl)acetyl]piperidine;
(36) 1-(2-{4-[2-(3,5-di-tert-butyl-4-methoxyphenyl)-1,3-thiazol-4-yl]piperidin-1-yl}-2-oxoethyl)-3-methylimidazolidin-2-one;
(37) 3-(2-{4-[2-(3,5-di-tert-butyl-4-methoxyphenyl)-1,3-thiazol-4-yl]piperidin-1-yl}-2-oxoethyl)-1,3-oxazolidin-2-one;
(38) 4-[2-(3,5-di-tert-butyl-4-methoxyphenyl)-1,3-thiazol-4-yl]-1-[(3,5-dimethyl-1H-pyrazol-1-yl)acetyl]piperidine;
(39) 4-[2-(3,5-di-tert-butyphenyl)-1,3-thiazol-4-yl]-1-[(3,5-dimethyl-1H-pyrazol-1-yl)acetyl]piperidine;
(40) 1-(2-{4-[2-(3,5-di-tert-butyl-4-methoxyphenyl)-1,3-thiazol-4-yl]piperidin-1-yl}-2-oxoethyl)-1H-pyrrolo[2,3-b]pyridine;
(41) 1-{2-[4-(2-{3-tert-butyl-5-[(trifluoromethyl)thio]phenyl}-1,3-thiazol-4-yl)piperidin-1-yl]-2-oxoethyl}-1H-pyrrolo[2,3-b]pyridine;
(42) 1-(2-{4-[2-(3,5-di-tert-butyl-4-methoxyphenyl)-1,3-thiazol-4-yl]piperidin-1-yl}-2-oxoethyl)-1H-benzimidazole;
(43) 1-(2-{4-[2-(3,5-di-tert-butyl-4-methoxyphenyl)-1,3-thiazol-4-yl]piperidin-1-yl}-2-oxoethyl)-2-methyl-1H-benzimidazole;
(44) 2-[4-(2-{3-tert-butyl-5-[(trifluoromethyl)thio]phenyl}-1,3-thiazol-4-yl)piperidin-1-yl]-2-oxoethyl}-1H-benzimidazole;
(45) 1-{2-[4-(2-{3-tert-butyl-5-[(trifluoromethyl)thio]phenyl}-1,3-thiazol-4-yl)piperidin-1-yl]-2-oxoethyl}-2-methyl-1H-benzimidazole;
(46) 4-[2-(3,5-di-tert-butylphenyl)-1,3-thiazol-4-yl]-1-{[5-methyl-3-(trifluoromethyl)-1H-pyrazol-1-yl]acetyl}piperidine;
(47) 1-{[3,5-bis(trifluoromethyl)-1H-pyrazol-1-yl]acetyl}-4-[2-(3,5-di-tert-butylphenyl)-1,3-thiazol-4-yl]piperidine;
(48) 4-bromo-3,5-dimethyl-1H-pyrazol-1-yl)acetyl]-4-[2-(3,5-di-tert-butylphenyl)-1,3-thiazol-4-yl]piperidine;
(49) 1-[(4-iodo-3,5-dimethyl-1H-pyrazol-1-yl)acetyl]-4-[2-(3,5-di-tert-butylphenyl)-1,3-thiazol-4-yl]piperidine;
(50) 1-(2-{4-[2-(3,5-di-tert-butyl-4-methoxyphenyl)-1,3-thiazol-4-yl]piperidin-1-yl}-2-oxoethyl)imidazolidin-2-one;
(51) [1-(2-{4-[2-(3,5-di-tert-butyl-4-methoxyphenyl)-1,3-thiazol-4-yl]piperidin-1-yl}-2-oxoethyl)-1H-imidazol-2-yl]metanol;
(52) [1-(2-{4-[2-(3,5-di-tert-butyl-4-methoxyphenyl)-1,3-thiazol-4-yl]piperidin-1-yl}-2-oxoethyl)-1H-benzimidazol-2-yl]metanol;
(53) 4-[2-(3,5-di-tert-butyl-4-methoxyphenyl)-1,3-thiazol-4-yl]-1-{[2-(trifluoromethyl)-1H-imidazol-1-yl]acetyl}piperidine;
(54) 4-[2-(3,5-di-tert-butyl-4-methoxyphenyl)-1,3-thiazol-4-yl]-1-{[2-methyl-1H-imidazol-1-yl]acetyl}piperidine;
(55) 4-[2-(3,5-di-tert-butyl-4-methoxyphenyl)-1,3-thiazol-4-yl]-1-(1H-imidazol-1-ylacetyl)piperidine;
(56) [1-(2-{4-[2-(3,5-di-tert-butylphenyl)-1,3-thiazol-4-yl]piperidin-1-yl}-2-oxoethyl)-5-methyl-1H-pyrazol-3-yl]acetic acid;
(57) Potassium (1-{2-[4-(2-{3-tert-butyl-5-[(trifluoromethyl)thiol]phenyl}-1,3-thiazol-4-yl)piperidin-1-yl]-2-oxoethyl}-5-methyl-1H-pyrazol-3-yl)acetate;
(58) 4-[2-(3,5-di-tert-butylphenyl)-1,3-thiazol-4-yl]-1-[(2,4-dimethyl-1H-imidazol-1-yl)acetyl]piperidine;
(59) 1-(2-{4-[2-(3,5-di-tert-butylphenyl)-1,3-thiazol-4-yl]piperidin-1-yl}-2-oxoethyl) -3-ethyl-1,3-dihydro-2H-imidazol-2-one;
(60) 3-[2-(4-{2-[3,5-bis(trifluoromethyl)phenyl]-1,3-thiazol-4-yl}piperidin-1-yl)-2-oxoethyl]-3H-imidazo[4,5-b]pyridine;
(61) 4-{2-[3,5-bis(trifluoromethyl)phenyl]-1,3-thiazol-4-yl}-1-[(3,5-dimethyl-1H-1,2,4-triazol-1-yl)acetyl]piperidine;
(62) 3-[2-(4-{2-[3,5-bis(trifluoromethyl)phenyl]-1,3-thiazol-4-yl}piperidin-1-yl)-2-oxoethyl]-3H-imidazo[4,5-c]pyridine;
(63) 1-[2-(4-{2-[3,5-bis(trifluoromethyl)phenyl]-1,3-thiazol-4-yl}piperidin-1-yl)-2-oxoethyl]-1H-imidazo[4,5-c]pyridine;
(64) 1-[2-(4-{2-[3,5-bis(trifluoromethyl)phenyl]-1,3-thiazol-4-yl}piperidin-1-yl)-2-oxoethyl]-2-methyl-1H-imidazo[4,5-c]pyridine;
(65) 1-(2-{4-[2-(3,5-di-tert-butyl-4-methoxyphenyl)-1,3-thiazol-4-yl]piperidin-1-yl}-2-oxoethyl)-1H-imidazole-2-carboxylic acid dihydrochloride;
(66) 1-ethyl-3-{2-oxo-2-[4-(2-phenyl-1,3-thiazol-4-yl)piperidin-1-yl]ethyl}-1,3-dihydro-2H-imidazol-2-one;
(67) 1-ethyl-3-(2-{4-[2-(4-methoxyphenyl)-1,3-thiazol-4-yl]piperidin-1-yl}-2-oxoethyl)-1,3-dihydro-2H-imidazol-2-one;
(68) 1-(2-{4-[2-(4-tert-butylphenyl)-1,3-thiazol-4-yl]piperidin-1-yl}-2-oxoethyl)-3-ethyl-1,3-dihydro-2H-imidazol-2-one;
(69) 1-(2-{4-[2-(4-chlorophenyl)-1,3-thiazol-4-yl]piperidin-1-yl}-2-oxoethyl)-3-ethyl-1,3-dihydro-2H-imidazol-2-one;
(70) 1-(2-{4-[2-(4-bromophenyl)-1,3-thiazol-4-yl]piperidin-1-yl}-2-oxoethyl)-3-ethyl-1,3-dihydro-2H-imidazol-2-one;
(71) 1-(2-{4-[2-(4-trifluoromethylphenyl)-1,3-thiazol-4-yl]piperidin-1-yl}-2-oxoethyl)-3-ethyl-1,3-dihydro-2H-imidazol-2-one;
(72) 1-(2-{4-[2-(3-chlorophenyl)-1,3-thiazol-4-yl]piperidin-1-yl}-2-oxoethyl)-3-ethyl-1,3-dihydro-2H-imidazol-2-one;
(73) 1-(2-{4-[2-(3-bromophenyl)-1,3-thiazol-4-yl]piperidin-1-yl}-2-oxoethyl)-3-ethyl-1,3-dihydro-2H-imidazol-2-one;
(74) 1-(2-{4-[2-(3-trifluoromethylphenyl)-1,3-thiazol-4-yl]piperidin-1-yl}-2-oxoethyl)-3-ethyl-1,3-dihydro-2H-imidazol-2-one;
(75) 1-(2-{4-[2-(3,5-dichlorophenyl)-1,3-thiazol-4-yl]piperidin-1-yl}-2-oxoethyl)-3-ethyl-1,3-dihydro-2H-imidazol-2-one; and
(76) 1-ethyl-3-{2-oxo-2-[4-(2-pyridin-4-yl-1,3-thiazol-4-yl)piperidin-1-yl]ethyl}-1,3-dihydro-2H-imidazol-2-one.

The invention also encompasses a pharmaceutical composition comprising a compound of Formula I in combination with a pharmaceutically acceptable carrier.

The invention also encompasses a method for treating a disease or condition mediated by the CXCR3 chemokine receptor comprising administering to a patient in need of such treatment a therapeutically effective amount of a compound of Formula I.

The invention also encompasses a method for treating a disease or condition mediated by the CXCR3 chemokine receptor comprising administering to a patient in need of such treatment a therapeutically effective amount of a compound of Formula I, wherein the disease or condition is selected from the group consisting of: acute and chronic transplant rejection, psoriasis, rheumatoid arthritis and multiple sclerosis.

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl and 1,1-dimethylethyl.

The term “cycloalkyl” means mono-, bi- or tri-cyclic structures, optionally combined with linear or branched structures, having the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, cyclobutylmethyl, cyclopropylmethyl 1-methylcyclopropyl and the like.

### Optical Isomers—Diastereomers—Geometric Isomers—Tautomers

Some of the compounds described herein may exists as mixtures of tautomers. The term “tautomers” embraces the standard meaning of the term, i.e. a type of isomerism in which two or more isomers are rapidly interconverted so that they ordinarily exist together in equilibrium. Tautomers include, e.g., compounds that undergo facile proton shifts from one atom of the compound to another atom of the compound. Some of the compounds described herein may exist as tautomers with different points of attachment of hydrogen. Such an example might be a ketone and its enol form known as keto-enol tautomers or an amide and its hydroxy imine tautomer. The individual tautomers of the compounds of Formula I, as well as mixtures thereof, are included in the scope of this invention. By way of illustration, tautomers included in this definition include, but are not limited to:

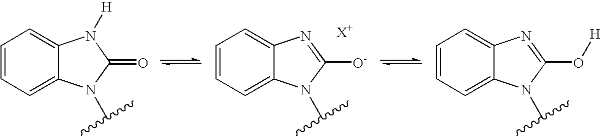

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Utilities

The compounds of the present invention are modulators of CXCR3 chemokine receptor function and are of use in antagonizing chemokine mediated cell signalling and in particular are of use in the prophylaxis and/or treatment of diseases or disorders involving inappropriate T-cell trafficking. The invention extends to such a use and to the use of the compounds of Formula I for the manufacture of a medicament for treating such diseases and disorders. Particular diseases include inflammatory, autoimmune and immunoregulatory disorders.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases or conditions of humans or other species which can be treated with compounds of Formula I, include, but are not limited to: autoimmune mediated inflammatory or allergic diseases and conditions, including respiratory diseases such as asthma, particularly bronchial asthma, systemic lupus erythematosus, ankylosing spondylitis, systemic sclerosis, autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; acute and chronic graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis); vasculitis (e.g., necrotizing, cutaneous, and hypersensitivity vasculitis); cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, and polymyositis.

The compounds of the present invention are accordingly useful in treating, preventing, ameliorating, controlling or reducing the risk of a wide variety of inflammatory and immunoregulatory disorders and diseases as well as autoimmune pathologies. In a specific embodiment, the present invention is directed to the use of the subject compounds for treating, preventing, ameliorating, controlling or reducing the risk of autoimmune diseases, such as rheumatoid arthritis, multiple sclerosis, psoriasis or psoriatic arthritis.

In another aspect, the instant invention may be used to evaluate putative Specific agonists or antagonists of chemokine receptors, including CXCR3. Accordingly, the present invention is directed to the use of these compounds in the preparation and execution of screening assays for compounds which modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CXCR3. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for treating CXCR3 mediated diseases in humans and animals comprising combining a compound of the present invention with a pharmaceutical carrier or diluent.

In a preferred aspect of the present invention, a subject compound may be used in a method of inhibiting the binding of a chemokine to a chemokine receptor, such as CXCR3, of a target cell, which comprises contacting the target cell with an amount of the compound which is effective at inhibiting the binding of the chemokine to the chemokine receptor.

The subject treated in the methods above is a mammal, preferably a human being, male or female, in whom modulation of chemokine receptor activity is desired. “Modulation” as used herein is intended to encompass antagonism, agonism, partial antagonism, inverse agonism and/or partial agonism. In a preferred aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

### Dose Ranges

The magnitude of prophylactic or therapeutic dose of a compound of Formula I will, of course, vary with the nature and severity of the condition to be treated, and with the particular compound of Formula I used and its route of administration. The dose will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0.1 mg to about 10 mg) of a compound of Formula I per kg of body weight per day.

In the case where an oral composition is employed, a suitable dosage range is, e.g. from about 0.01 mg to about 100 mg of a compound of Formula I per kg of body weight per day, preferably from about 0.1 mg to about 10 mg per kg.

For use where a composition for sublingual administration is employed, a suitable dosage range is from 0.01 mg to about 25 mg (preferably from 0.1 mg to about 5 mg) of a compound of Formula I per kg of body weight per day.

### Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or non-aqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206, as well as natalizumab; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) immunomodulaltory antibody therapies including anti-TNF therapies such as Etanercept (Enbrel®), Infliximab (Remicade®), Adalimumab (Humira®) or other TNF peptide or receptor sequestrants; Efalizumab (Raptiva®), Daclizumab (Zenapax®), Basiliximab (Simulect®), Rituximab (Rituxan®), visilizumab (Nuvion®), Abatacept (Orencia®) or other interleukin peptide or receptor binding antibodies; (e) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (f) non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (g) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac and zidometacin), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine, olsalazine, mesalamine and balsalazide) and the pyrazolones (apazone, bezpiperylon, feprazone, mofebutazone, oxyphenbutazone, phenylbutazone); (h) cyclooxygenase-2 (COX-2) inhibitors such as celecoxib, rofecoxib, and parecoxib; (i) inhibitors of phosphodiesterase type IV (PDE-TV); (j) antagonists of the other chemokine receptors, especially CCR1, CCR2, CCR5 and CCR3; (k) cholesterol lowering agents such as HMG-CoA reductase inhibitors (lovastatin, simvastatin, pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (1) anti-diabetic agents such as insulin, sulfonylureas, biguanides (metformin), a-glucosidase inhibitors (acarbose), glitazars (muraglitazar) and glitazones (troglitazone, pioglitazone, englitazone, MCC-555, BRL49653 and the like); (m) preparations of interferon beta (Avonex®, Rebif®, interferon beta-1a, Betaseron®, interferon beta-1b); (n) anticholinergic agents such as muscarinic antagonists (ipratropium and tiatropium); (o) current treatments for multiple sclerosis, including prednisolone, glatiramer, deoxyadenosine, mitoxantrone, methotrexate, and cyclophosphamide; (p) p38 kinase inhibitors; (q) DMARDs, such as methotrexate, leflunamide or plaquenil; (r) other compounds such as 5-aminosalicylic acid and prodrugs thereof, antimetabolites such as azathioprine, mycophenolate and 6-mercaptopurine, cytotoxic cancer chemotherapeutic agents and cytokine sequestrants.

The weight ratio of the compound of the Formula I to the second active ingredient may be varied and will depend upon the effective dose of each ingredient. Generally, an effective dose of each will be used. Thus, for example, when a compound of the Formula I is combined with an NSAID the weight ratio of the compound of the Formula I to the NSAID will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Methods of Synthesis

The following abbreviations are used in the synthetic schemes:

Ac is acetyl [CH₃C(O)—]; Ac₂O is acetic anhydride; 9-BBN is 9-borabicyclo[3.3.1]nonane; Bn is benzyl; DIAD is diisopropylazodicarboxylate; DIBAL is diisobutylaluminum hydride; DMF is N,N-dimethylformamide; DMSO is dimethyl sulfoxide; EDAC (or EDC) is 1-ethyl-3-[3-(dimethylamino)propyl]-carbodiimide HCl; Et₃N is triethylamine; Et is ethyl; EtOAc is ethyl acetate; EtOH is ethanol; HCl is hydrochloric acid; HOBt is 1-hydroxybenzotriazole; HPLC is high performance liquid chromatography; LG is leaving group; M is molar; mmol is millimole; Me is methyl; MeOH is methanol; MsCl methanesulfonyl chloride; N is normal; NaHMDS is sodium hexamethyldisiliazide; NaOAc is sodium acetate; NaOtBu is sodium tert-butoxide; NMO is N-methylmorpholine N oxide; PG is protecting group; Pd(dba)₂ is tris(dibenzylideneacetone)dipalladium; PdCl₂(Ph₃P)₂ is dichloro bis-(triphenylphosphine)palladium; Ph is phenyl; PhMe is toluene; PPh₃ is triphenylphosphine; PMB is para-methoxybenzyl; RT is room temperature; TBAF is tetrabutyl ammonium fluoride; TBS is tert-butyldimethylsilyl; tBu is tert-butyl; Tf is triflate; TFA is trifluoroacetic acid; THF is tetrahydrofuran; TLC is thin layer chromatography; TMS is trimethylsilyl; TPAP is tetrapropylammonium perruthenate;

### General Schemes

These compounds are prepared by the convergent assembly of three molecular fragments. The synthesis of these fragments is depicted in Schemes 1, 2 and 3 below.

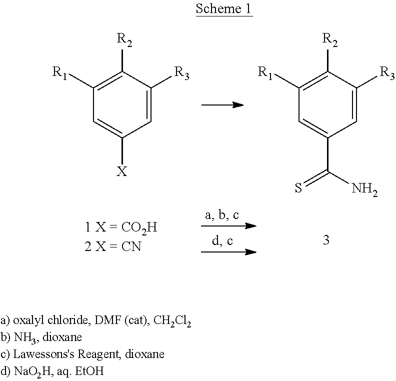

Using known methods substituted thioamides 3 are prepared from their corresponding benzoic acids 1 or benzonitriles 2 as shown in Scheme 1. Benzoic acids 1 are converted to their acid chlorides and ammonolyzed to the amide. The amide is converted to the thioamide with Lawesson's reagent. Benzonitiles 2 are converted to their amides by partial hydrolysis and then to the thioamides 3 as above.

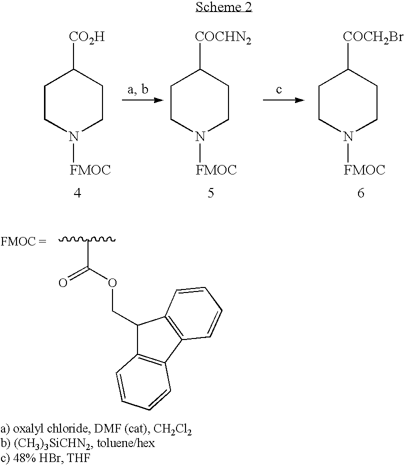

Bromoketone 6 is prepared from N-FMOC isonipecotic acid 4 as shown in Scheme 2. Acid 4 is converted to its acid chloride and exposed to trimethylsilyldiazomethane, giving the diazoketone 5. Decomposition of 5 with 48% HBr gives the bromoketone 6.

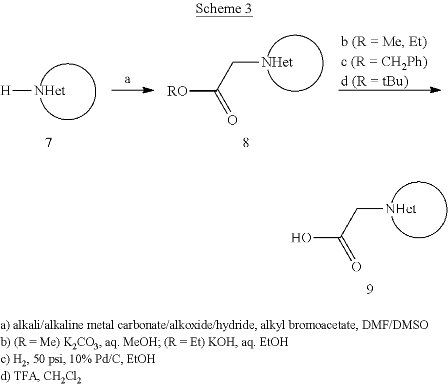

The heterocyclic acetic acids 9 are prepared from the corresponding known heterocycles 7 as shown in Scheme 3. Exposure of 7 to a bromoacetic acid ester in the presence of alkali or alkaline metal bases in polar aprotic solvents affords the esters 8. Basic hydrolysis, reduction or solvolysis as appropriate gives the desired 9.

Antagonists I are assembled as shown in Scheme 4.

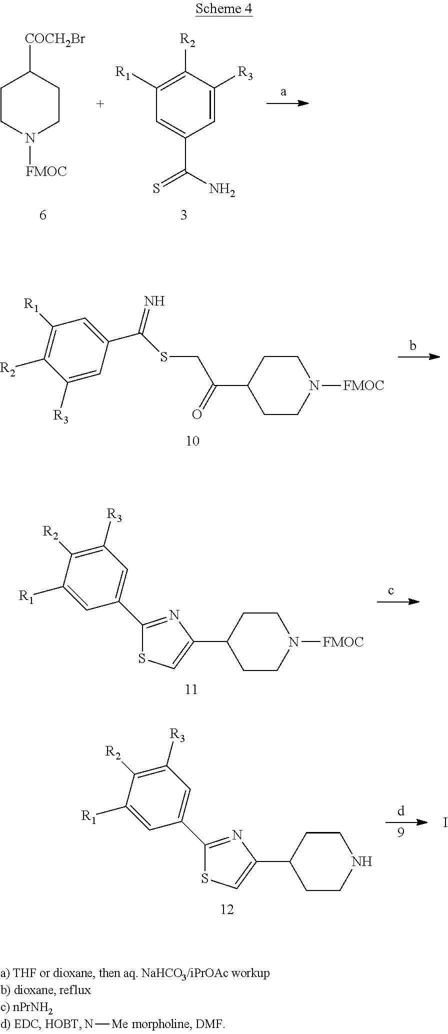

Combination of 6 and 3 in ethereal solvents such as THF or dioxane followed by basic workup affords the imino-thioether 10. Thermal cyclization gives the thiazole 11. Removal of the FMOC protecting group gives the piperidine 12. Amide formation using acids 9 affords the target antagonists I.

Specific examples are described below.

### Example 1

### Step A 9H-fluoren-9-ylmethyl-4-(chlorocarbonyl)piperidine-1-carboxylate

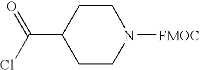

A 0° solution of 1-[(9H-fluoren-9-ylmethoxy)carbonyl]piperidine-4-carboxylic acid (FMOC-isonipecotic acid; 25.01 g; 71.17 mmol) in dry CH₂Cl₂ (225 mL) was treated dropwise with oxalyl chloride (8.69 mL; 99.64 mmol). The cold bath was removed and the solution stirred at ambient temperature for 3 h. The solution was recovered and evaporated to an oil, then flushed with toluene (2×50 mL). The residue was used without further purification.

### Step B 9H-fluoren-9-ylmethyl-4-(diazoacetyl)piperidine-1-carboxylate

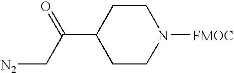

A solution of 9H-fluoren-9-ylmethyl-4-(chlorocarbonyl)piperidine-1-carboxylate in toluene (125 mL) was added dropwise to a 0° solution of trimethylsilyldiazomethane (144 mL; 2M in hexane; 288 mmol). The cold bath was removed and the solution stirred at ambient temperature for 16 h. The solution was recovered and evaporated to a residue that was used without further purification. ¹H NMR (CDCl₃): δ 7.80 (d, 2H, J=7.5 Hz), 7.60 (d, 2H, J=7.5 Hz), 7.43 (t, 2H, J=7.3 Hz), 7.35 (dt, 2H, Jₜ=7.6 Hz, J_{d}=0.9 Hz), 4.47 (bs, 2H), 4.28 (t, 1H, J=6.8 Hz), 4.23 (vbs, 1H), 4.17 (vbs, 1H), 2.91 (vbs, 3H), 2.77 (vbs, 1H), 1.92-1.60 (bmult, 4H).

### Step C 9H-fluoren-9-ylmethyl-4-(bromoacetyl)piperidine-1-carboxylate

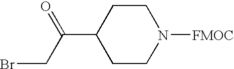

A 0° solution of 9H-fluoren-9-ylmethyl-4-(diazoacetyl)piperidine-1-carboxylate in THF (150 mL) was treated dropwise with 48% HBr (50 mL). The mixture was warmed to ambient temperature and partitioned between water (100 mL) and isopropyl acetate (500 mL). The organic was dried over MgSO₄, filtered and evaporated to a heavy oil. The crude product was chromatographed over silica gel (50% to 100% CH₂Cl₂/hexane; linear gradient), affording the title bromoketone as a white solid (27.88 g). ¹H NMR (CDCl₃): δ 7.81 (d, 2H, J=7.4 Hz), 7.60 (d, 2H, J=7.4 Hz), 7.44 (t, 2H, J=7.3 Hz), 7.37 (t, 2H, J=7.2 Hz), 4.50 (vbs, 2H), 4.26 (t, J=6.7 Hz), 4.22 (vbs, 1H), 4.04 (vbs, 1H), 4.00 (s, 2H), 2.93 (bmult, 3H), 1.91 (vbs, 2H), 1.60 (vbs, 2H).

### Step D 3,5-di-tert-butyl-4-methoxybenzonitrile

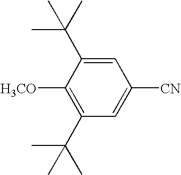

A solution of 3,5-di-tert-butyl-4-hydroxybenzonitrile (15.13 g; 65.40 mmol) in dry DMF (200 mL) was sequentially treated with methyl iodide (12.22 mL; 196.2 mmol) and cesium carbonate (29.83 g; 91.55 mmol). The mixture was stirred for 8 h. The reaction mixture was diluted with isopropyl acetate (500 mL) and washed with water (3×200 mL). The organic was dried over MgSO₄, filtered and evaporated to a solid (16.12 g) that was used without further purification. ¹H NMR (CDCl₃): δ 7.80 (s, 2H), 3.79 (s, 3H), 1.49 (s, 18H).

### Step E 3,5-di-tert-butyl-4-methoxybenzamide

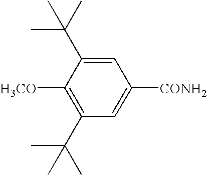

A solution of 3,5-di-tert-butyl-4-methoxybenzonitrile (16.12 g; 65.40 mmol based on 100% yield above) in ethanol (300 mL) was treated with an aqueous solution of sodium hydroperoxide (65.4 mL; 2M; 130.8 mmol). The solution was stirred at ambient temperature for 16 h, during which a white solid precipitated. The reaction mixture was diluted with isopropyl acetate (500 mL) and washed with water (100 mL). The organic was dried over MgSO₄, filtered and evaporated to a white solid. The solid was digested in methylcyclohexane (100 mL) with stirring at reflux, then cooled to ambient. Filtration afforded the title compound as a white solid (15.97 g). ¹H NMR (CDCl₃): δ 7.78 (s, 2H), 6.02 (vbs, 1H), 5.71 (vbs, 1H), 3.76 (s, 3H), 1.48 (s, 18H).

### Step F 3,5-di-tert-butyl-4-methoxybenzthioamide

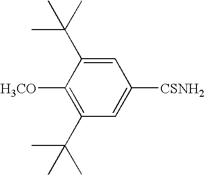

A solution of 3,5-di-tert-butyl-4-methoxybenzamide (2235 g; 84.86 mmol) in dioxane (250 mL) was treated with Lawesson's Reagent (51.49 g; 127.3 mmol). The mixture was stirred at ambient temperature for 16 h. The now yellow mixture was filtered and the filtrate concentrated to an oil. The crude product was chromatographed over silica gel (0% to 100% CH₂Cl₂/hexane; linear gradient). The yellow product fractions were combined and evaporated to give the title compound as a yellow solid (23.44 g). ¹H NMR (CDCl₃): δ 7.80 (s, 2H), 7.62 (vbs, 1H), 7.17 (vbs, 1H), 3.74 (s, 3H), 1.42 (s, 18H).

### Step G 9H-fluoren-9-ylmethyl-4-[2-(3,5-di-tert-butyl-4-methoxyphenyl)-1,3-thiazol-4-yl]piperidine-1-carboxylate

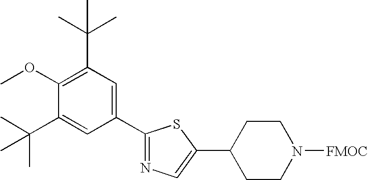

A solution of 3,5-di-tert-butyl-4-methoxybenzthioamide (15.26 g; 56.61 mmol) in dioxane (425 mL) was treated in portions with 9H-fluoren-9-ylmethyl-4-(bromoacetyl)piperidine-1-carboxylate (23.40 g; 54.63 mmol) at ambient temperature. The bromoketone dissolved in 15 min. The reaction mixture was stirred for 3 h during which it became a thick suspension of solid. The solid was recovered by filtration and pull-dried in the filtering funnel until it was sufficiently granular to enable facile removal. The solid was transferred to a 5 L motor stirred flask containing isopropyl acetate (2 L). The mixture was vigorously stirred for 1 h until the mixture was a fine solid suspension. Saturated aqueous sodium bicarbonate (1 L) was added and the mixture stirred until dissolution was complete (about 10 min). The organic was recovered and dried over MgSO₄. Filtration and concentration afforded a heavy oil (33.40 g). The crude product (consisting of a mixture of the title compound and its imino thioether precursor) was dissolved in dioxane (250 mL) and refluxed for 45 min. The solvent was evaporated to give the title compound (32.04 g) which was used without further purification. ¹H NMR (CD₃OD): δ 7.84 (s, 2H), 7.81 (d, 2H, J=7.4 Hz), 7.64 (d, 2H, J=7.4 Hz), 7.41 (bt, 2H, J=7.3 Hz), 7.37 (bt, 2H, J=7.2 Hz), 7.13 (s, 1H), 4.61 (vbs, 3H), 4.29 (t, 1H, J=6.7 Hz), 3.76 (s, 3H), 3.68 (s, 2H), 3.00 (vbt, 11H), 2.93 (bmult, 1H), 2.13-1.90 (vbmult, 2H, 1.49 (s, 18H). LRMS calc: 608.3 obs: 609.4 (M+H).

### Step H 4-[2-(3,5-di-tert-butyl-4-methoxyphenyl)-1,3-thiazol-4-yl]piperidine

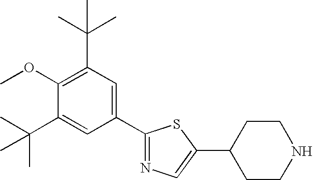

A solution of 9H-fluoren-9-ylmethyl-4-[2-(3,5-di-tert-butyl-4-methoxyphenyl)-1,3-thiazol-4-yl]piperidine-1-carboxylate (32.04 g; 52.70 mmol) in CH₂Cl₂ (250 mL) was added dropwise to n-propylamine (250 mL) at ambient temperature. The solution was stirred for 1 h. The reaction was evaporated to about ¼ its volume and applied to a silica gel column (2 L c.v.; packed with 40:1 CH₂Cl₂/MeOH). The column was eluted without fractionation (40:1 CH₂Cl₂/MeOH) until the title compound appeared in the eluant (TLC R_{f}=0 with 20:1 CH₂Cl₂/MeOH). Fractionation was initiated and continued until all more mobile products no longer appeared in the eluant. The eluant was switched (100:10:1 CH₂Cl₂/MeOH/Et₃N) and elution continued without fractionation until the title compound was recovered. Mixed fractions were rechromatographed using the same method, affording the title compound (16.91 g). ¹H NMR (CD₃OD): δ 7.80 (s, 2H), 7.12 (s, 1H), 3.71 (s, 3H), 3.23 (dt, 2H, Jₜ=12.6, J_{d}=1.3 Hz), 3.00 (tt, 1H, J=11.8, 3.5 Hz), 2.85 (dt, 2H, Jₜ=12.4, J_{d}=2.4 Hz), 2.12 (bd, 2H, J=13.4 Hz), 1.78 (dquart, 2H, J_{q}=12.5, J_{d}=4.0 Hz), 1.46 (s, 18H).

### Step J 3H-imidazo[4,5-b]pyridine-3-ylacetic acid benzyl ester

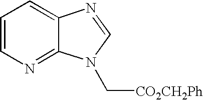

A solution of 4-azabenzimidazole (13.68 g; 114.8 mmol) in dry DMF (350 mL) was sequentially treated with benzyl bromoacetate (18.01 mL; 114.8 mmol) and cesium carbonate (37.40 g; 114.8 mmol) at ambient temperature. The mixture was stirred for 1.5 h. The reaction was diluted with isopropyl acetate (700 mL) and filtered. The filtrate was washed with water (3×350 mL). The organic was dried over MgSO₄, filtered and evaporated to an oil. The crude product was chromatographed over silica gel (700 mL c.v.; EtOAc) affording the title compound (12.70 g). ¹H NMR (CDCl₃): δ 8.40 (dd, 1H, J=4.6, 1.3 Hz), 8.13 (s, 1H), 8.11 (dd, 1H, J=7.9, 1.3 Hz), 7.39-7.35 (mult, 3H), 7.34-7.32 (mult, 2H), 7.28 (dd, 1H, J=8.0, 4.7 Hz), 5.23 (s, 2H), 5.13 (s, 2H).

### Step K 3H-imidazo[4,5-b]pyridine-3-ylacetic acid

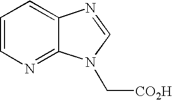

A solution of 3H-imidazo[4,5-b]pyridine-3-ylacetic acid benzyl ester (12.70 g; 47.52 mmol) in ethanol (150 mL) was treated with 10% Pd/C hydrogenation catalyst (5.08 g). The mixture was shaken under a hydrogen atmosphere (50 psi) for 5 h. The mixture was poured into methanol (500 mL) and stirred for 30 min. The mixture was filtered through Celite and the filtrate concentrated to a pink solid (9.13 g). The solid was recrystallized from ethanol (100 mL) to afford the title compound as a white solid (7.79 g). ¹H NMR (CD₃OD) δ 8.42 (s, 1H), 8.40 (dd, 1H, J=4.6, 1.3 Hz), 8.14 (dd, 1H, J=7.9, 1.3 Hz), 7.38 (dd, 1H, J=8.0, 4.7 Hz), 5.19 (s, 2H).

### Step L 3-(2-{4-[2-(3,5-di-tert-butyl-4-methoxyphenyl)-1,3-thiazol-4-yl]piperidin-1-yl}-2-oxoethyl)-3H-imidazo[4,5-b]pyridine

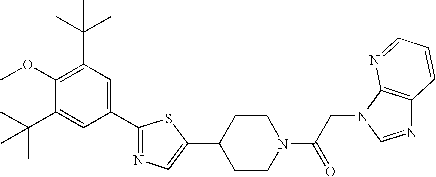

A solution of 4-[2-(3,5-di-tert-butyl-4-methoxyphenyl)-1,3-thiazol-4-yl]piperidine (5.70 g; in dry DMF (50 mL) was treated sequentially with 3H-imidazo[4,5-b]pyridine-3-ylacetic acid (3.91 g; 22.06 mmol), 4-methylmorpholine (3.23 mL; 29.41 mmol), 1-hydroxybenzotriazole hydrate (3.97 g; 29.41 mmol) and EDC (5.64 g; 29.41 mmol). The solution was stirred at ambient temperature for 3 h. Saturated aqueous sodium bicarbonate (75 mL) was added dropwise to the reaction with vigorous stirring. The precipitated solid was recovered by filtration (8.67 g). The solid was added to vigorously stirred water (450 mL) and heated to reflux momentarily. Cooling to ambient and filtration afforded the title compound (7.95 g). ¹H NMR (CD₃OD): δ 8.37 (dd, 1H, J=4.8, 1.3 Hz), 8.35 (s, 1H), 8.10 (dd, 1H, J=8.0, 1.3 Hz), 7.81 (s, 2H), 7.35 (dd, 1H, J=8.0, 4.8 Hz), 7.16 (s, FM 5.42 (½AB, 1H, J=17.0 Hz), 5.36 (½AB, 1H, J=16.9 Hz), 4.55 (bd, 1H, J=13.3 Hz), 4.20 (bd, 1H, J=13.4 Hz), 3.71 (s, 3H), 3.42 (bt, 1H, J=12.2 Hz), 3.15 (tt, 1H, J=11.6, 3.2 Hz), 2.90 (bt, 1H, J=11.9 Hz), 2.24 (bd, 1H, J=13.2 Hz), 2.12 (bd, 1H, J=13.3 Hz), 1.94 (dquart, 1H, J_{q}=12.7, J_{d}=3.7 Hz), 1.73 (dquart, 1H, J_{q}=12.6, J_{d}=3.6 Hz), 1.47 (s, 18H). LRMS calc: 545.3 obs: 546.4 (M+H).

### Example 2

### Step A 2,4-dimethyl-1H-imidazol-1-ylacetic acid tert-butyl ester and 2,5-dimethyl-1H-imidazol-1-ylacetic acid tert-butyl ester

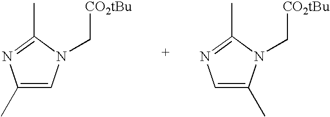

A solution of 2,4-dimethylimidazole (8.40 g; 87.38 mmol) in dry DMF (250 mL) was treated with potassium tert-butoxide (9.806 g; 87.38 mmol). The mixture was stirred at ambient temperature until homogenous. tert-Butyl bromoacetate (15.29 mL; 104.9 mmol) was added dropwise. The solution was stirred for 15 min, then diluted with isopropyl acetate (400 mL) and washed with pH7 phosphate buffer (3×250 mL). The organic was dried over MgSO₄, filtered and evaporated to an oil. The crude product (consisting of the two title compounds) was chromatographed over silica gel (0% to 10% MeOH/CH₂Cl₂; linear gradient). All fractions containing the two products were combined. The residue was rechromatographed on Chiralcel OD stationary phase (Daicel Chemical Industries Ltd., Chiralcel Technologies Inc.; 10% ethanol/heptane; λ=220 nM). The more mobile 2,5 isomer and less mobile 2,4 isomer were obtained. ¹H NMR (CDCl₃): (2,4 isomer) δ 6.49 (s, 1H), 439 (s, 2H), 2.24 (s, 3H), 2.17 (s, 3H), 1.42 (s, 9H); (2,5 isomer) δ 6.67 (s, 1H), 4.41 (s, 2H), 2.35 (s, 3H), 2.15 (s, 3H), 1.48 (s, 9H).

### Step B 2,4-dimethyl-1H-imidazol-1-ylacetic acid hydrotrifluoroacetate

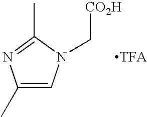

A solution of 2,4-dimethyl-1H-imidazol-1-ylacetic acid tert-butyl ester (1.07 g; 5.09 mmol) in CH₂Cl₂ (15 mL) was treated with trifluoroacetic acid (30 mL) at ambient temperature. The solution was stirred for 1 h. The volatiles were removed and the residue flushed with toluene (2×15 mL) affording the title compound as an oil (1.32 g). ¹H NMR (CD₃OD): δ 7.20 (s, 1H), 5.03 (s, 2H), 2.59 (s, 3H), 2.32 (s, 3H).

### Step C 4-[2-(3,5-di-tert-butyl-4-methoxyphenyl)-1,3-thiazol-4-yl]-1-[(2,4-dimethyl-1H-imidazol-1-yl)acetyl]piperidine

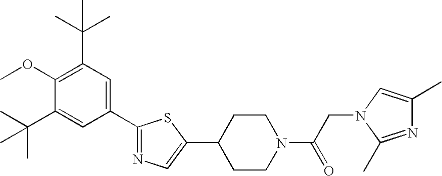

Using the method of Example 1, Step L and starting with the products of Example 1, Step H and Example 2, Step B the title compound was obtained. ¹H NMR (CD₃OD): δ 7.88 (s, 2H), 7.21 (s, 1H), 6.67 (bs, 1H), 5.03 (½AB, 1H, J=17.0 Hz), 4.96 (½AB, 1H, J=17.0 Hz), 4.59 (bd, 1H, J=13.4 Hz), 4.11 (bd, 1H, J=12.9 Hz), 3.76 (s, 3H), 3.37 (bt, 1H, J=12.9 Hz), 3.15 (tt, 1H, J=11.4, 3.8 Hz), 2.88 (bt, 1H, J=12.8 Hz), 2.26 (s, 3H), 2.21 (bd, 1H, J=12.9 Hz), 2.16 (bd, 1H, J=15.0 Hz), 2.11 (s, 3H), 1.84 (dquart, 1H, J_{q}=12.5, J_{d}=3.5 Hz), 1.72 (dquart, 1H, J_{q}=12.7, J_{d}=3.7 Hz), 1.49 (s, 18H). LRMS calc: 522.3 obs: 523.4 (M+H).

### Step D 2,5-dimethyl-1H-imidazol-1-ylacetic acid hydrotrifluoroacetate

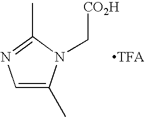

Using the method of Example 2, Step B with 2,5-dimethyl-1H-imidazol-1-ylacetic acid tert-butyl ester (Example 2, Step A) as the starting material the title compound was obtained.

### Step E 4-[2-(3,5-di-tert-butyl-4-methoxyphenyl)-1,3-thiazol-4-yl]-1-[(2,5-dimethyl-1H-imidazol-1-yl)acetyl]piperidine

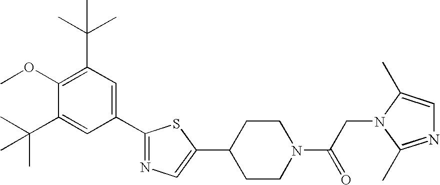

Using the method of Example 1, Step L and starting with the products of Example 1, Step H and Example 2, Step D the title compound was obtained. ¹H NMR (CDCl₃): δ 7.79 (s, 2H), 6.85 (s, 1H), 6.73 (vbs, 1H), 4.72 (vbs, 2H), 4.64 (bd, 1H, J=13.6 Hz), 3.98 (bd, 1H, J=13.5 Hz), 3.70 (s, 3H), 3.34 (bt, 1H, J=12.3 Hz), 3.11 (bt, 1H, J=11.6 Hz), 2.85 (bt, 1H, J=12.0 Hz), 2.40 (bs, 3H), 2.30 (bd, 1H, J=13.2 Hz), 2.20-2.15 (bs overlapping bd, 4H), 1.76 (bpent, 2H, J=12.6 Hz), 1.47 (s, 18H). LRMS calc: 522.3 obs: 523.4 (M+H).

### Example 3

### Step A (2-methyl-1H-imidazol-5-yl)methanol hydrochloride

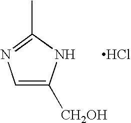

A solution of dihydroxyacetone (1.905 g; 21.16 mmol) in aqueous ammonia (28%; 15 mL) was placed in a microwavable vessel. Acetamidine hydrochloride (2.00 g; 21.16 mmol) was added, the vessel sealed and the solution heated via microwave irradiation at 120° for 10 min. The solution was lyophilized. The derived residue was digested in refluxing acetone (100 mL), cooled to ambient temperature and filtered. The filtrate was evaporated and the residue dissolved in aqueous HCl (50 mL; 1N). The solution was lyophilized to a heavy oil. The oil was flushed with methanol (2×20 mL), then diluted with acetone (100 mL) and allowed to stand at −10° for 8 hours. The supernatant was decanted and the crystallized solid rinsed with cold acetone, affording the title compound (1.25 g). ¹H NMR (CD₃OD): δ 7.30 (s, 1H), 4.61 (s, 2H), 2.62 (s, 3H).

### Step B [4-(hydroxymethyl)-2-methyl-1H-imidazol-1-yl]acetic acid methyl ester and [5-(hydroxymethyl)-2-methyl-1H-imidazol-1-yl]acetic acid methyl ester

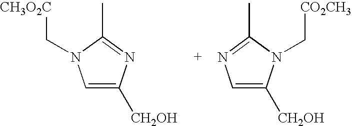

A solution of (2-methyl-1H-imidazol-5-yl)methanol hydrochloride (990 mg; 6.66 mmol) in dry DMF (20 mL) was sequentially treated with cesium carbonate (5.43 g; 16.66 mmol) and methyl bromoacetate (633 μL; 6.66 mmol). The mixture was stirred at ambient temperature for 16 h. The reaction was diluted with isopropyl acetate (100 mL) and filtered. The filtrate was concentrated and flushed with xylene (3×50 mL; 1 mm vacuum; 60° bath). The residue was chromatographed on Chiralcel OJ stationary phase (15% ethanol/heptane, λ=220 nM). The more mobile 5-hydroxymethyl and less mobile 4-hydroxymethyl title isomers were obtained. ¹H NMR (CD₃OD): δ 6.93 (bs, 1H), 4.82 (s, 2H), 4.46 (s, 2H), 3.77 (s, 3H), 2.29 (s, 3H).

### Step C Potassium [4-(hydroxymethyl)-2-methyl-1H-imidazol-1-yl]acetate

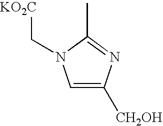

A solution of [4-(hydroxymethyl)-2-methyl-1H-imidazol-1-yl]acetic acid methyl ester (58 mg; 0.315 mmol) in methanol (2 mL) was treated with aqueous potassium carbonate (315 μL; 1M; 0.315 mmol). The solution was stirred at ambient temperature for 24 h. All volatiles were removed and the resultant solid digested in hot ethanol (5 mL). The mixture was cooled to ambient and filtered. The filtrate was evaporated to give the title compound. ¹H NMR (CD₃OD): δ 6.87 (bs, 1H), 4.44 (s, 2H), 4.42 (s, 2H), 2.29 (s, 3H).

### Step D [1-(2-{4-[2-(3,5-di-tert-butyl-4-methoxyphenyl)-1,3-thiazol-4-yl]piperidin-1-yl}-2-oxoethyl)-2-methyl-1H-imidazol-4-yl]methanol

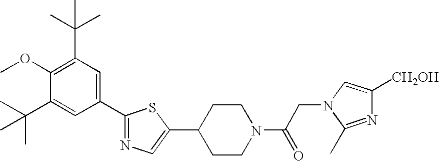

Using the method of Example 1, Step L and starting with the products of Example 1, Step H and Example 3, Step C the title compound was obtained. ¹H NMR (CD₃OD): δ 7.80 (s, 2H), 7.16 (s, 1H), 6.99 (s, 1H), 5.09 (½AB, 1H, J=17.4 Hz), 5.03 (½AB, 1H, J=17.3 Hz), 4.58 (bd, 1H, J=12.6 Hz), 4.49 (bs, 2H), 4.03 (bd, 1H, J=13.2 Hz), 3.72 (s, 3H), 3.34 (bt, 1H, J=12.1 Hz), 3.13 (tt, 1H, J=11.8, 4.0 Hz), 2.90 (bt, 1H, J=11.8 Hz), 2.34 (s, 3H), 2.20 (bd, 1H, J=12.6 Hz), 2.13 (bd, 1H, J=13.2 Hz), 1.85 (dquart, 1H, J_{q}=12.6, 3.7 Hz), 1.71 (dquart, 1H, J_{q}=12.8, J_{d}=3.5 Hz), 1.46 (s, 18H). LRMS calc: 538.3 obs: 539.3 (M+H).

### Step E Potassium [5-(hydroxymethyl)-2-methyl-1H-imidazol-1-yl]acetate

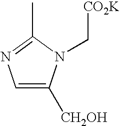

Using the method of Example 3, Step C with [5-(hydroxymethyl)-2-methyl-1H-imidazol-1-yl]acetic acid methyl ester (Example 3, Step B) as starting material the title compound was obtained.

### Step F [1-(2-{4-[2-(3,5-di-tert-butyl-4-methoxyphenyl)-1,3-thiazol-4-yl]piperidin-1-yl}-2-oxoethyl)-2-methyl-1H-imidazol-5-yl]methanol

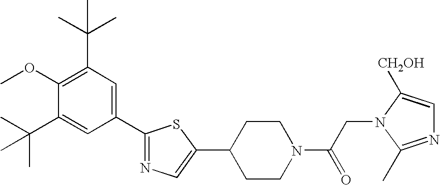

Using the method of Example 1, Step L and starting with the products of Example 1, Step H and Example 3, Step E the title compound was obtained. ¹H NMR (CD₃OD): δ 7.81 (s, 2H), 7.16 (s, 1H), 6.77 (s, 1H), 5.08 (½AB, 1H, J=17.6 Hz), 5.01 (½AB, 1H, J=17.4 Hz), 4.57 (bd, 1H, J=13.7 Hz), 4.47 (bs, 2H), 4.03 (bd, 1H, J=12.8 Hz), 3.72 (s, 3H), 3.37 (bt, 1H, J=13.6 Hz), 3.14 (bt, 1H, J=11.7 Hz), 2.90 (bt, 1H, J=12.4 Hz), 2.29 (s, 3H), 2.22 (bd, 1H, J=12.6 Hz), 2.14 (bd, 1H, J=12.5 Hz), 1.87 (dquart, 1H, J_{q}=12.7, J_{d}=4.0 Hz), 1.71 (dquart, 1H, J_{q}=12.8, J_{d}=4.0 Hz), 1.47 (s, 18H). LRMS calc: 538.3 obs: 539.3 (M+H).

### Example 4

### Step A 1-ethyl-1,3-dihydro-2H-imidazol-2-one

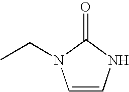

A solution of aminoacetaldehyde dimethyl acetal (2.72 g; 22.86 mmol) in dry THF (50 mL) was treated with ethyl isocyanate (1.81 mL; 22.86 mmol). The solution was stirred at ambient temperature for 15 min. The reaction was evaporated and the residue dissolved in dry benzene (50 mL). Trifluoroacetic acid (10 mL) was added and the solution refluxed for 2 h. Cooling and evaporation gave a residue that was flushed with toluene (2×10 mL). The crude product was adsorbed onto silica gel (CH₂Cl₂) and eluted without fractionation (100:5:1 CH₂Cl₂/MeOH/Et₃N) to give the title compound (1.92 g). ¹H NMR (CD₃OD): δ 6.49 (d, 1H, J=2.9 Hz), 6.41 (d, 1H, J=3.0 Hz), 3.68 (quart, 2H, J=7.3 Hz), 1.29 (t, 3H, J=7.3 Hz).

### Step B (3-ethyl-2-oxo-2,3-dihydro-1H-imidazol-1-yl)acetic acid tert-butyl ester

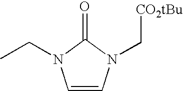

A solution of 1-ethyl-1,3-dihydro-2H-imidazol-2-one (4.26 g; 37.99 mmol) in dry DMF (100 mL) was sequentially treated with tert-butyl bromoacetate (5.54 mL; 37.99 mmol) and cesium carbonate (12.38 g; 37.99 mmol). The reaction was stirred at ambient temperature for 16 h. The reaction mixture was diluted with isopropyl acetate (300 mL) and washed with pH7 phosphate buffer (3×100 mL). The organic was dried over MgSO₄, filtered and evaporated to a residue. The crude product was chromatographed over silica gel (50% to 100% MTBE/hexane; linear gradient) to give the title compound (4.97 g). ¹H NMR (CD₃OD): δ 6.52 (d, 1H, J=2.7 Hz), 6.48 (d, 1H, J=2.7 Hz), 4.33 (s, 2H), 3.67 (quart, 2H, J=7.3 Hz), 1.47 (s, 9H), 1.27 (t, 3H, J=7.3 Hz).

### Step C (3-ethyl-2-oxo-2,3-dihydro-1H-imidazol-1-yl)acetic acid

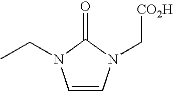

Using the method of Example 2, Step B with the product of Example 4, Step B as the starting material the title compound was obtained. ¹H NMR (CD₃OD): δ 6.52 (d, 1H, J=3.0 Hz), 6.49 (d, 1H, J=2.9 Hz), 4.41 (s, 2H), 3.67 (quart, 2H, J=7.2 Hz), 1.27 (t, 3H, J=7.2 Hz).

### Step D 1-(2-{4-[2-(3,5-di-tert-butyl-4-methoxyphenyl)-1,3-thiazol-4-yl]piperidin-1-yl}-2-oxoethyl)-3-ethyl-1,3-dihydro-2H-imidazol-2-one

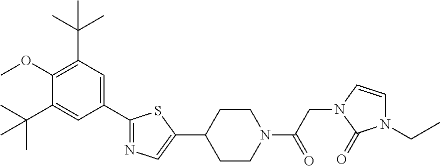

Using the method of Example 1, Step L and starting with the products of Example 1, Step H and Example 4, Step C the title compound was obtained. ¹H NMR (CD₃OD): δ 7.84 (s, 2H), 7.18 (s, 1H), 6.55 (d, 1H, J=3.0 Hz), 6.48 (d, 1H, J=3.0 Hz), 4.68 (½AB, 1H, J=16.7 Hz), 4.59 (½AB, 1H, J=16.8 Hz), 4.60 (bd, 1H, J=13.0 Hz), 4.09 (bd, 1H, J=13.9 Hz), 3.70 (quart, 2H, J=7.3 Hz), 3.31 (bt, 1H, J=12.0 Hz), 3.15 (tt, 1H, J=11.7, 3.2 Hz), 2.90 (bt, 1H, J=11.8 Hz), 2.22-2.14 (bmult, 2H), 1.83 (dquart, 1H, J_{q}=12.6, J_{d}=3.7 Hz), 1.73 (dquart, 1H, J_{q}=12.6, J_{d}=3.8 Hz), 1.50 (s, 18H), 1.30 (t, 3H, J=7.2 Hz). LRMS calc: 538.3 obs: 539.4 (M+H).

### Step E (3-methyl-2-oxo-2,3-dihydro-1H-imidazol-1-yl)acetic acid tert-butyl ester

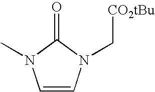

Using the method of Example 4, Step B with 1-methyl-1,3-dihydro-2H-imidazol-2-one as the starting material the title compound was obtained.

### Step F (3-methyl-2-oxo-2,3-dihydro-1H-imidazol-1-yl)acetic acid

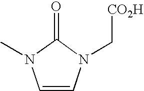

Using the method of Example 2, Step B with the product of Example 4, Step E as the starting material the title compound was obtained.

### Step G 1-(2-{4-[2-(3,5-di-tert-butyl-4-methoxyphenyl)-1,3-thiazol-4-yl]piperidin-1-yl}-2-oxoethyl)-3-methyl-1,3-dihydro-2H-imidazol-2-one

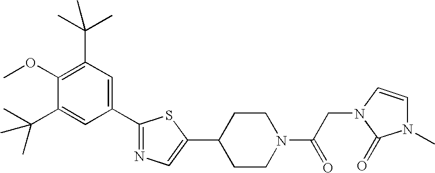

Using the method of Example 1, Step L and starting with the products of Example 1, Step H and Example 4, Step F the title compound was obtained. ¹H NMR (CD₃OD): δ 7.80 (s, 2H), 7.14 (s, 1H), 6.45 (d, 1H, J=3.0 Hz), 6.43 (d, 1H, J=3.0 Hz), 4.63 (½AB, 1H, J=16.9 Hz), 4.55 (½AB, 1H, J=16.8 Hz), 4.56 (bd, 1H, J=13.1 Hz), 4.04 (bd, 1H, J=13.7 Hz), 3.71 (s, 3H), 3.31 (dt, 1H, Jₜ=12.9, J_{d}=2.5 Hz), 3.25 (s, 3H), 3.10 (tt, 1H, J=11.7, 3.6 Hz), 2.85 (dt, 1H, Jₜ=12.9, J_{d}=2.6 Hz), 2.16 (bd, 1H, J=13.5 Hz), 2.10 (bd, 1H, J=13.3 Hz), 1.80 (dquart, 1H, J_{q}=12.5, J_{d}=4.2 Hz), 1.68 (dquart, 1H, J_{q}=12.6, J_{d}=4.3 Hz), 1.46 (s, 18H). LRMS calc: 524.3 obs: 525.3 (M+H).

### Example 5

### Step A N-Methylaminoacetone hydrochloride

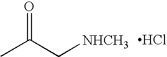

A solution of chloroacetone (1.07 mL; 13.42 mmol) in dry THF (50 mL) was sequentially treated with N-methyl benzylamine (1.63 g; 13.42 mmol), triethylamine (2.07 mL; 14.76 mmol) and tetrabutylammonium bromide (211 mg; 0.654 mmol). The solution was stirred at ambient temperature for 72 h. The now heterogenous mixture was filtered and evaporated to an oil (2.61 g). The oil was dissolved in ethanol (25 mL). Concentrated Ha was added (3 mL) followed by 10% Pd/C hydrogenation catalyst (560 mg). The mixture was shaken under a hydrogen atmosphere (50 psi) for 4 h. The mixture was filtered through Celite and the filtrate evaporated, then allowed to stand under high vacuum for 1 hour. The residue was flushed with acetone (2×30 mL) causing a solid to precipitate. Trituration from acetone gave the title compound (1.29 g).

### Step B 1,4-dimethyl-1,3-dihydro-2H-imidazol-2-one

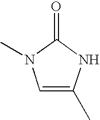

A solution of N-methylaminoacetone hydrochloride (462 mg; 3.74 mmol) in methanol (10 mL) was treated with KOCN (334 mg; 4.11 mmol). The mixture was refluxed 1 h. After cooling to ambient temperature, the mixture was filtered and the filtrate evaporated. The residue was adsorbed onto silica gel (CH₂Cl₂) and eluted without fractionation (20:1 CH₂Cl₂/MeOH). Evaporation of the eluant gave the title compound (351 mg).

### Step C (3,5-dimethyl-2-oxo-2,3-dihydro-1H-imidazol-1-yl)acetic acid tert-butyl ester

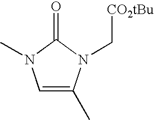

Using the method of Example 4, Step B with the product of Example 5, Step B the title compound was obtained.

### Step D (3,5-dimethyl-2-oxo-2,3-dihydro-1H-imidazol-1-yl)acetic acid

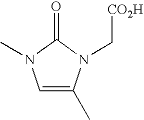

Using the method of Example 2, Step B with the product of Example 5, Step C the title compound was obtained.

### Step E 3-(2-{4-[2-(3,5-di-tert-butyl-4-methoxyphenyl)-1,3-thiazol-4-yl]piperidin-1-yl}-2-oxoethyl)-1,4-dimethyl-1,3-dihydro-2H-imidazol-2-one

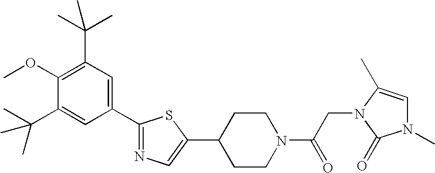

Using the method of Example 1, Step L with the products of Example 1, Step H and Example 5, Step D as starting materials the title compound was obtained. ¹H NMR (CD₃OD): δ 7.82 (s, 2H), 7.16 (s, 1H), 6.19 (d, 1H, J=1.1 Hz), 4.65 (½AB, 1H, J=17.2 Hz), 4.58 (½AB, 1H, J=17.0 Hz), 4.56 (bd, 1H, J=13.1 Hz), 4.10 (bd, 1H, J=13.9 Hz), 3.73 (s, 3H), 3.31 (dt, 1H, Jₜ=12.9, J_{d}=2.5 Hz), 3.22 (s, 3H), 3.13 (tt, 1H, J=11.7, 3.7 Hz), 2.88 (dt, 1H, Jₜ=12.6, J_{d}=2.3 Hz), 2.20 (bd, 1H, J=12.5 Hz), 2.13 (bd, 1H, J=12.6 Hz), 1.84 (dquart, 1H, J_{q}=12.6, J_{d}=4.1 Hz), 1.71 (dquart, 1H, J_{q}=12.6, J_{d}=4.3 Hz), 1.48 (s, 18H). LRMS: calc. 538.3 obs: 539.4 (M+H).

### Example 6

### Step A 3,3-dimethoxy-2-methylpropionic acid methyl ester

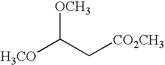

A solution of methyl 2-bromopropionate (23.92 g; 143.3 mmol) and trimethylorthoformate (18.8 mL; 171.9 mmol) in dry benzene (50 mL) was added dropwise to a refluxing suspension of powdered zinc (71.7 g; 1.096 mol) in dry benzene (150 mL). After the reactants were combined more zinc (17.9 g; 0.274 mol) was added and the mixture refluxed 6 h. The mixture was cooled to ambient temperature and decanted, then filtered through Celite. The filtrate was concentrated to a residue. The residue was fractionally distilled under high vacuum (1 mm). The fraction distilling at 45°-47° was recovered, giving the title compound (4.02 g).

### Step B 3-hydroxy-4-methylthiophene-2-carboxylic acid methyl ester

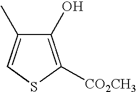

3,3-dimethoxy-2-methylpropionic acid methyl ester (4.02 g; 24.79 mmol) and methyl mercaptoacetate (5.26 g; 49.57 mmol) were combined and treated with BF₃ etherate (94 μL; 0.74 mmol). A short path distillation head was attached and the flask immersed in a 130° bath. Heating was continued until methanol stopped distilling (approx. 10 min). The residue was cooled to ambient and dissolved in methanolic sodium methoxide (62 mL; 1M). The solution was stirred for 16 h. The reaction was evaporated to a residue and partitioned between isopropyl acetate (200 mL) and aq. HCl (100 mL; 1N). The organic was dried over MgSO₄, filtered and concentrated. The crude product was chromatographed over silica gel (50% to 100% CH₂Cl₂/hexane; linear gradient) to give the title compound (3.15 g).

### Step C (4-methyl-1H-pyrazol-3-yl)acetic acid methyl ester hydrochloride

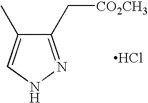

A solution of the product of Example 6, Step B (3.15 g; 18.29 mmol) in n-butanol (50 mL) was treated with hydrazine monohydrate (8.90 mL; 183 mmol). The solution was refluxed for 16 h. The reaction was concentrated to a residue and dissolved in aq. NaOH (75 mL; 2.5N). The solution was refluxed for 8 h. The solution was lyophilized and the resultant solids digested in hot ethanol (50 mL). The mixture was cooled to ambient and filtered. The filtrate was concentrated and dissolved in methanol (50 mL). Dry gaseous HCl was bubbled into the solution causing the precipitation of a solid. The mixture was stirred until it returned to ambient temperature, then filtered and the filtrate concentrated to afford the title compound (3.04 g).

### Step D tert-butyl methyl 2,2′-(4-methyl-1H-pyrazole-1,3-diyl)diacetate

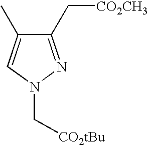

A solution of the product of Example 6, Step C (3.04 g; 15.95 mmol) in thy DMSO (50 mL) was treated with tert-butyl bromoacetate (3.49 mL; 23.92 mmol) followed by cesium carbonate (10.39 g; 31.89 mmol). The mixture was stirred at ambient temperature for 24 h. The mixture was diluted with isopropyl acetate (150 mL) and filtered. The filtrate was washed with pH7 phosphate buffer solution (3×50 mL). The organic was dried over MgSO₄, filtered and concentrated to a residue. The crude product, consisting of the title compound and its regioisomer, was chromatographed on Chiralcel OJ stationary phase (10% ethanol/heptane; λ=230 nM). The less mobile, major isomer was isolated affording the title compound (1.21 g).

### Step E [3-(2-methoxy-2-oxoethyl)-4-methyl-1H-pyrazol-1-yl]acetic acid hydrotrifluoroacetate

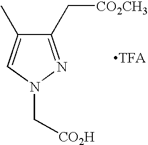

Using the method of Example 2, Step B and the product of Example 6, Step D the title compound was obtained.

### Step F [1-(2-{4-[2-(3,5-di-tert-butyl-4-methoxyphenyl)-1,3-thiazol-4-yl]piperidin-1-yl}-2-oxoethyl)-4-methyl-1H-pyrazol-3-yl]acetic acid methyl ester

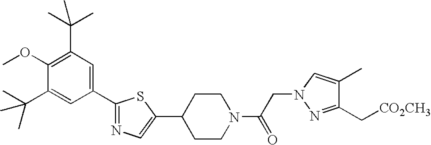

Using the method of Example 1, Step L with the products of Example 1, Step H and the product of Example 6, Step E the title compound was obtained.

### Step G [1-(2-{4-[2-(3,5-di-tert-butyl-4-methoxyphenyl)-1,3-thiazol-4-yl]piperidin-1-yl}-2-oxoethyl)-4-methyl-1H-pyrazol-3-yl]acetate bis(hydrotrifluoroacetate)

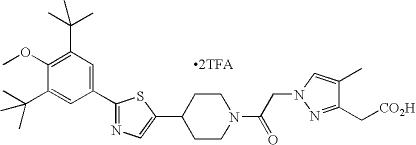

A solution of the product of Example 6, Step F (127 mg; 0.219 mmol) in methanol (4 mL) was treated with aq. K₂CO₃ (590 μL; 1M; 0.59 mmol). The solution was stirred at ambient temperature for 24 h. All volatiles were removed and the residue digested in hot ethanol (5 mL). The mixture was cooled to ambient temperature and filtered. The filtrate was evaporated and the derived solid purified on C8 reverse phase HPLC (CH₃CN/H₂O/0.1% TFA). Evaporation of the appropriate fractions afforded the title compound (120 mg). ¹H NMR (CD₃OD): δ 7.81 (s, 2H), 7.47 (vbs, 1H), 7.21 (s, 1H), 5.15 (½AB, 11H, J=16.4 Hz), 5.06 (½AB, 1H, J=16.6 Hz), 4.57 (bd, 1H, J=12.9 Hz), 4.04 (bd, 1H, J=13.3 Hz), 3.71 (s, 3H), 3.64 (½AB, 1H, J=15.7 Hz), 3.59 (½AB, 1H, J=15.5 Hz), 3.31 (bt, 1H, J=11.0 Hz), 3.13 (tt, 1H, J=11.8, 3.6 Hz), 2.85 (bt, 1H, J=11.9 Hz), 2.14 (bd, 1H, J=15.5 Hz), 2.11 (bd, 1H, J=15.7 Hz), 2.04 (s, 3H), 1.76 (dquart, 1H, J_{q}=12.6, J_{d}=3.6 Hz), 1.66 (dquart, 1H, J_{q}=12.4, J_{d}=3.3 Hz), 1.46 (s, 3H). LRMS calc: 566.3 obs: 567.4 (M+H).

### Example 7

### Step A 4-methyl-2-(methylthio)-1H-imidazole

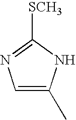

A solution of 2-mercapto-4-methyl-1H-imidazole hydrochloride (prepared using the method of Smith, et. al. JCS 1951, p. 2217; 320 mg; 2.80 mmol) in dry acetone (6 mL) was treated with potassium carbonate (774 mg; 5.60 mmol) followed by methyl iodide (174 μL; 2.80 mmol). The mixture was stirred at ambient temperature for 8 h. The mixture was diluted with isopropyl acetate (20 mL) and washed with pH7 phosphate buffer solution (20 mL). The organic was dried over MgSO₄, filtered and evaporated to give the title compound (352 mg).

### Step B [4-methyl-2-(methylthio)-1H-imidazol-1-yl]acetic acid methyl ester

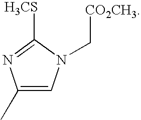

A solution of 4-methyl-2-(methylthio)-1H-imidazole (352 mg; 2.75 mmol) in dry DMF (8 mL) was treated sequentially with cesium carbonate (895 mg; 2.75 mmol) and methyl bromoacetate (261 μL; 2.75 mmol). The mixture was stirred at ambient temperature for 16 h. The reaction mixture was diluted with isopropyl acetate (30 mL) and filtered. The filtrate was washed with pH7 phosphate buffer solution (3×10 mL). The organic was dried over MgSO₄, filtered and evaporated to a residue. The crude product was chromatographed over silica gel (30:1 CH₂Cl₂/MeOH). The derived mixture of isomers (163 mg) was rechromatographed on Chiralcel OJ stationary phase (10% ethanol/heptane; λ=220 nM). The major, more mobile product was isolated affording the title compound (80 mg).

### Step C [4-methyl-2-(methylsulfonyl)-1H-imidazol-1-yl]acetic acid methyl ester

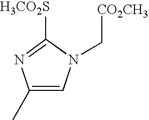

A solution of [4-methyl-2-(methylthio)-1H-imidazol-1-yl]acetic acid methyl ester (80 mg; 0.40 mmol) in CH₂Cl₂ (3 mL) was treated with mCPBA (276 mg; titer=75 wt %; 1.20 mmol). The mixture was stirred at ambient temperature for 16 h. The mixture was filtered and evaporated. The derived solids were slurried in methanol and filtered. The filtrate was evaporated to give a residue (132 mg) which was chromatographed over silica gel (30:1 CH₂Cl₂/MeOH). The major product was recovered, dissolved in isopropyl acetate and washed once with aq. sodium bicarbonate. Drying, filtration and evaporation afforded the title compound (51 mg).

### Step D [4-methyl-2-(methylsulfonyl)-1H-imidazol-1-yl]acetic acid hydrochloride

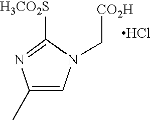

A solution of [4-methyl-2-(methylsulfonyl)-1H-imidazol-1-yl]acetic acid methyl ester (51 mg; 0.22 mmol) in methanol (2 mL) was treated with aq. potassium carbonate (220 μL; 0.22 mmol). The solution was stirred at ambient temperature for 16 h. The volatiles were removed and the resultant solids digested in hot ethanol (5 mL). The mixture was allowed to cool to ambient and filtered, then diluted with aq. HCl (2 mL; 1N). Lyophilization afforded the title compound (46 mg).

### Step E 4-[2-(3,5-di-tert-butyl-4-methoxyphenyl)-1,3-thiazol-4-yl]-1-{4-methyl-2-(methylsulfonyl)-1H-imidazol-1-yl]acetyl}piperidine

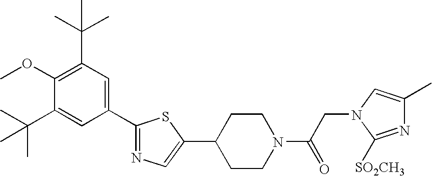

Using the method of Example 1, Step L with the products of Example 1, Step H and Example 7, Step D as starting materials the title compound was obtained. ¹H NMR (CDCl₃): δ 7.81 (s, 2H), 6.88 (s, 1H), 6.75 (s, 1H), 5.20 (½AB, 1H, J=16.5 Hz), 5.11 (½AB, 1H, J=16.4 Hz), 4.62 (bd, 1H, J=13.2 Hz), 3.86 (bd, 1H, J=13.3 Hz), 3.72 (s, 3H), 3.32 (bt, 1H, J=12.1 Hz), 3.26 (s, 3H), 3.13 (vbt, 1H, J=11.3 Hz), 2.89 (bt, 1H, J=11.8 Hz), 2.27-2.25 (s overlapping bd, 4H total), 2.21 (bd, 1H, J=11.8 Hz), 2.19 (bd, 1H, J=12.8 Hz), 1.84 (dquart, 1H, J_{q}=12.6, J_{d}=3.5 Hz), 1.74 (dquart, 1H, J_{q}=12.6, J_{d}=3.5 Hz), 1.48 (s, 18H). LRMS calc: 586.3 obs: 587.4 (M+H).

### Example 8

### Step A (2-oxo-2,3-dihydro-1H-imidazol-1-yl)acetic acid ethyl ester

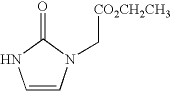

Using the method of Example 4, Step A with ethyl isocyanatoacetate in place of ethyl isocyanate as one of the starting materials the title compound was obtained.

### Step B Potassium (2-oxo-2,3-dihydro-1H-imidazol-1-yl)acetate

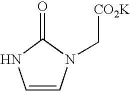

A solution of (2-oxo-2,3-dihydro-1H-imidazol-1-yl)acetic acid ethyl ester (180 mg; 1.06 mmol) in ethanol (5 mL) was treated with aq. KOH (1.06 mL; 1M). The solution was stirred at ambient temperature for 6 h. All volatiles were removed to give the title compound.

### Step C 1-(2-{4-[2-(3,5-di-tert-butyl-4-methoxyphenyl)-1,3-thiazol-4-yl]piperidin-1-yl)-2-oxoethyl)-1,3-dihydro-2H-imidazol-2-one

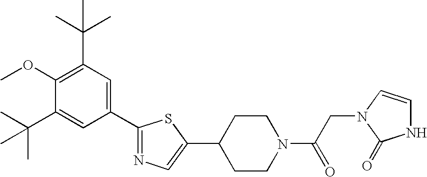

Using the method of Example 1, Step L with the products of Example 1, Step H and Example 8, Step B as starting materials the title compound was obtained. ¹H NMR (CD₃OD): δ 7.82 (s, 2H), 7.15 (s, 1H), 6.43 (d, 1H, J=3.0 Hz), 6.41 (d, 1H, J=3.0 Hz), 4.64 (½AB, 1H, J=16.7 Hz), 4.56 (½AB, 1H, J=16.7 Hz), 4.60 (bd, 1H, J=13.1 Hz), 4.07 (bd, 1H, J=13.2 Hz), 3.73 (s, 3H), 3.31 (dt, 1H, Jₜ=12.9, J_{d}=2.5 Hz), 3.12 (tt, 1H, J=11.6, 3.6 Hz), 2.87 (dt, 1H, Jₜ=13.0, J_{d}=2.5 Hz), 2.18 (bd, 1H, J=12.7 Hz), 2.13 (bd, 1H, J=12.6 Hz), 1.81 (dquart, 1H, J_{q}=12.5, J_{d}=4.2 Hz), 1.70 (dquart, 1H, J_{q}=12.6, J_{d}=4.3 Hz), 1.49 (s, 18H). LRMS calc: 510.3 obs: 511.3 (M+H).

### Example 9

### Step A tert-butyl ethyl 2,2′-(2-oxo-1H-imidazole-1,3-diyl)diacetate

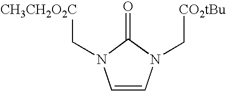

Using the method of Example 4, Step B with the product of Example 8, Step A as starting material the title compound was obtained. Silica gel chromatography was carried out using 40:1 CH₂Cl₂/MeOH.

### Step B [3-(2-ethoxy-2-oxoethyl)-2-oxo-2,3-dihydro-1H-imidazol-1-yl]acetic acid

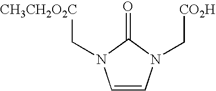

Using the method of Example 2, Step B with the product of Example 9, Step A as starting material the title compound was obtained.

### Step C [3-(2-{4-[2-(3,5-di-tert-butyl-4-methoxyphenyl)-1,3-thiazol-4-yl]piperidin-1-yl}-2-oxoethyl)-2-oxo-2,3-dihydro-1H-imidazol-1-yl]acetic acid ethyl ester

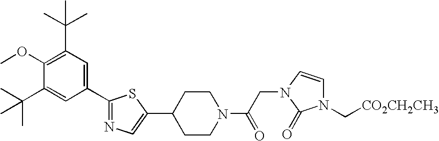

Using the method of Example 1, Step L with the products of Example 1, Step H and Example 9, Step B as starting materials the title compound was obtained.

### Step D Potassium [3-(2-{4-[2-(3,5-di-tert-butyl-4-methoxyphenyl)-1,3-thiazol-4-yl]piperidin-1-yl}-2-oxoethyl)-2-oxo-2,3-dihydro-1H-imidazol-1-yl]acetate

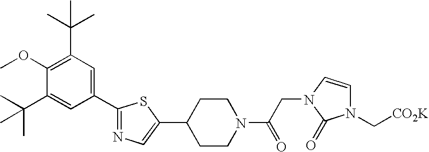

A solution of the product of Example 9, Step C (52 mg; 0.087 mmol) in ethanol/DMF (2.5 mL; 4:1) was treated with aq. KOH (122 μL; 1M). The solution was stirred at ambient temperature for 16 h. The solution was flushed with xylene (3×10 mL; 1 mm vacuum; 60° bath). The derived solids were digested in hot ethanol (5 mL), allowed to cool and filtered. The filtrate was evaporated and the resulting solid triturated with MTBE (10 mL) affording the title compound (47 mg). ¹H NMR (CD₃OD): δ 7.82 (s, 2H), 7.16 (s, 1H), 6.48 (d, 1H, J=2.8 Hz), 6.43 (d, 1H, J=2.9 Hz), 4.66 (½AB, 1H, J=16.7 Hz), 4.58 (½AB, 1H, J=16.8 Hz), 4.60 (bd, 1H, J=13.1 Hz), 4.20 (s, 2H), 4.07 (bd, 1H, J=14.4 Hz), 3.73 (s, 3H), 3.31 (dt, 1H, Jₜ=12.9, J_{d}=2.5 Hz), 3.12 (tt, 1H, J=11.7, 3.6 Hz), 2.87 (dt, 1H, Jₜ=13.0, J_{d}=2.3 Hz), 2.18 (bd, 1H, J=13.5 Hz), 2.12 (bd, 1H, J=12.0 Hz), 1.82 (dquart, 1H, J_{q}=12.9, J_{d}=4.2 Hz), 1.70 (dquart, 1H, J_{q}=12.6, J_{d}=4.3 Hz), 1.48 (s, 18H). LRMS calc: 568.3 obs: 569.3 (M+H).

### Example 10

### Step A 1-phenylimidazolidin-2-one

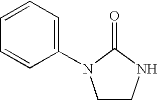

A solution of 2-chloroethylamine hydrochloride (270 mg; 2.33 mmol) in dry DMF (3 mL) was treated sequentially with phenyl isocyanate (254 μL; 2.33 mmol) and cesium carbonate (758 mg; 2.33 mmol). The mixture was stirred at ambient temperature for 16 h. The mixture was treated with potassium tert-butoxide (261 mg; 2.33 mmol) and stirred an additional 2 h. The reaction was diluted with pH7 phosphate buffer (10 mL) and filtered. The derived solid was chromatographed over silica gel (40:1 CH₂Cl_{2/}MeOH) to afford the title compound (195 mg).

### Step B (2-oxo-3-phenylimidazolidin-1-yl)acetic acid tert-butyl ester

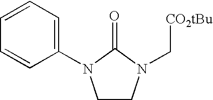

Using the method of Example 4, Step B with the product of Example 10, Step A the title compound was obtained. Isolation was effected by silica gel chromatography (40:1 CH₂Cl₂/MeOH).

### Step C (2-oxo-3-phenylimidazolidin-1-yl)acetic acid

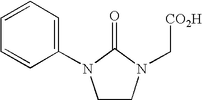

Using the method of Example 2, Step B and the product of Example 10, Step B the title compound was obtained.

### Step D 1-(2-{4-[2-(3,5-di-tert-butyl-4-methoxyphenyl)-1,3-thiazol-4-yl]piperidin-1-yl}-2-oxoethyl)-3-phenylimidazolidin-2-one

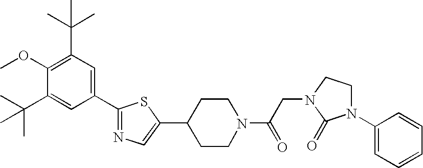

Using the method of Example 1, Step L and the products of Example 1, Step I-1 and Example 10, Step C as the starting materials the title compound was obtained. ¹H NMR (CD₃OD): δ 7.82 (s, 2H), 7.55 (d, 2H, J=8.0 Hz), 7.33 (t, 2H, J=7.9 Hz), 7.17 (s, 1H), 7.05 (t, 1H, J=7.5 Hz), 4.62 (bd, 1H, J=13.1 Hz), 4.28 (½AB, 1H, J=16.9 Hz), 4.19 (½AB, 1H, J=16.7 Hz), 4.06 (bd, 1H, J=13.9 Hz), 3.94 (t, 2H, J=8.1 Hz), 3.74 (s, 3H), 3.64 (t, 2H, J=8.4 Hz), 3.30 (dt, 1H, Jₜ=14.8, J_{d}=2.3 Hz), 3.13 (tt, 1H, J=11.9, 3.6 Hz), 2.88 (bt, 1H, J=11.9 Hz), 2.19 (bd, 1H, J=13.1 Hz), 2.14 (bd, 1H, J=12.9 Hz), 1.82 (dquart, 1H, J_{q}=12.6, J_{d}=3.6 Hz), 1.72 (dquart, 1H, J_{q}=12.4, J_{d}=3.5 Hz), 1.48 (s, 18H). LRMS calc: 588.3 obs: 589.3 (M+H).

### Example 11

### Step A 2-iodo-4-tert-butylaniline

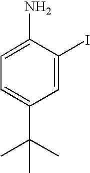

A solution of 4-tert-butylaniline (75.00 g; 0.503 mol) in methanol (140 mL) was treated with water (140 mL) and potassium carbonate (72.93 g; 0.528 mol). A solution of iodine monochloride (528 mL; 1M in CH₂Cl₂) was added dropwise to the reaction mixture and stirred at ambient temperature for 2 h. The dark solution was recovered and evaporated. Isopropyl acetate (2L) was added and the organic washed with water until the aqueous layer was clear. The organic was dried over MgSO₄, filtered and evaporated onto silica gel. The silica gel was exhaustively eluted (3:1 hexane/MTBE) without fractionation. The eluant was evaporated giving the partially purified title compound which was used as is (121.4 g).

### Step B 2-iodo-4-tert-butylnitrobenzene

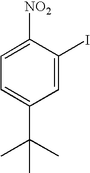

A solution of 2-iodo-4-tert-butylaniline (121.4 g; 0.442mol) in CH₂Cl₂ (4.2L) was treated at ambient temperature with mCPBA (305 g; titer=75 wt %; 1.326 mol) in portions. The mixture was stirred for 16 h. The thick suspension was filtered and evaporated. Isopropyl acetate (3L) was added and the organic washed with saturated aq. sodium bicarbonate until all mCBA was removed from the organic (by LC analysis; 5×750 mL). The organic was dried over MgSO₄, filtered and evaporated onto silica gel. The silica gel was exhaustively eluted (6:1 hexane/MTBE) without fractionation. The eluant was evaporated giving the partially purified title compound which was used as is (82.46 g).

### Step C 2-trifluoromethylthio-4-tert-butylnitrobenzene

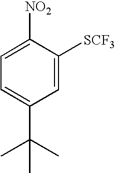

A solution of 2-iodo-4-tert-butylnitrobenzene (76.89 g; 0.252 mol) in dry NMP (830 mL) was treated with CuSCF₃ (31.08 g; 0.189 mol). The mixture was heated to 160° for 1 h. After cooling to ambient, the reaction mixture was diluted with isopropyl acetate (2L) and filtered. The filtrate was washed with pH7 phosphate buffer solution (3×1L). The organic was dried over MgSO₄, filtered and evaporated onto silica gel. The silica gel was exhaustively eluted (10:1 hexane/MTBE) without fractionation. The eluant was evaporated giving the partially purified title compound which was used as is (57.28 g).

### Step D 2-trifluoromethylthio-4-tert-butylaniline

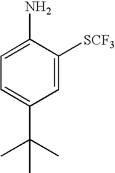

A solution of 2-trifluoromethylthio-4-tert-butylnitrobenzene (57.28 g; 0.205 mol) in methanol (570 mL) was treated with powdered zinc (134.9 g; 2.05 mol). Acetic acid (145 mL) was added dropwise through an attached condenser. The mixture was stirred for 1 h at ambient temperature. The reaction mixture was filtered through Celite and the filtrate concentrated. The resulting residue was slurried in CH₂Cl₂ (500 mL) and refiltered through Celite. The filtrate was evaporated onto silica gel. The silica gel was exhaustively eluted (10:1 hexane/MTBE) without fractionation. The eluant was concentrated to an oil. The oil was chromatographed over silica gel (2L c.v.; 20:1 hexane/MTBE) to give the title compound (16.04 g).

### Step E 2-trifluoromethylthio-4-tert-butyl-6-bromoaniline

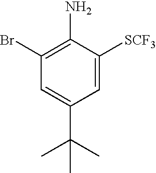

A solution of 2-trifluoromethylthio-4-tert-butylaniline (16.04 g; 64.34 mmol) in CH₂Cl₂ (300 mL) was treated with NBS (11.45 g; 64.34 mmol) in portions. The mixture was stirred at ambient temperature for 45 min. The reaction was recovered and evaporated to a residue. The crude product was digested in hot methylcyclohexane (320 mL). The mixture was cooled to ambient and filtered. The filtrate was evaporated to give the title compound (21.2 g).

### Step F 3-trifluoromethylthio-5-tert-butyl-bromobenzene

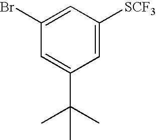

A 70° solution of isoamyl nitrite (17.2 mL; 129.2 mmol) in DMF (10 mL) was treated dropwise with a solution of 2-trifluoromethylthio-4-tert-butyl-6-bromoaniline (21.2 g; 64.6 mmol) in DMF (110 mL) over a 1 h period. Heating was continued for an additional 1.5 h. The reaction was diluted with isopropyl acetate (500 mL) and washed with pH7 phosphate buffer solution (3×200 mL). The organic was dried over MgSO₄, filtered and evaporated onto silica gel. The silica gel was placed atop a packed silica gel column and eluted (800 mL c.v.; hexane) to give the title compound as a pale rose colored oil (13.0 g).

### Step G 3-trifluoromethylthio-5-tert-butyl-benzonitrile

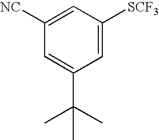

A solution of 3-trifluoromethylthio-5-tert-butyl-bromobenzene (313 mg; 1.00 mmol) in NMP (5 mL) was treated with CuCN (143 mg; 1.60 mmol). The mixture was heated at 150° for 1 h. The reaction was cooled to ambient temperature and diluted with isopropyl acetate (30 mL). The organic was washed with pH7 phosphate buffer solution (3×15 mL). The organic was dried over MgSO₄, filtered and concentrated to an oil. The oil was adsorbed onto silica gel (CH₂Cl₂). The silica gel was loaded onto a packed silica gel column and eluted (100 mL c.v.; 1:1 hexane/CH₂Cl₂) affording the title compound (232 mg).

### Step H 3-trifluoromethylthio-5-tert-butyl-benzamide

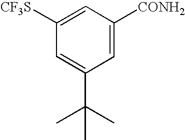

Using the method of Example 1, Step E and the product of Example 11, Step G the title compound was obtained.

### Step J 3-trifluoromethylthio-5-tert-butyl-benzthioamide

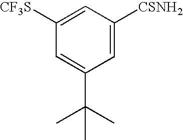

Using the method of Example 1, Step F and the product of Example 11, Step H the title compound was obtained.

### Step K 9H-fluoren-9-ylmethyl-4-(2-{3-tert-butyl-5-[(trifluoromethyl)thiolphenyl}-1,3-thiazol-4-yl)piperidine-1-carboxylate

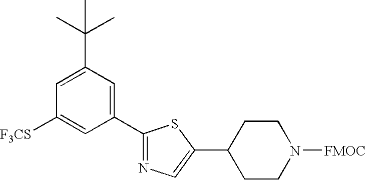

Using the method of Example 1, Step G and the products of Example 1, Step C and Example 11, Step J as the starting materials the title compound was obtained.

### Step L 4-(2-{3-tert-butyl-5-[(trifluoromethyl)thio]phenyl}-1,3-thiazol-4-yl)piperidine

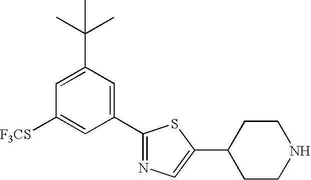

Using the method of Example 1, Step H with the product of Example 11, Step K the title compound was obtained.

### Step M 3-{2-[4-(2-{3-tert-butyl-5-{(trifluoromethyl)thio]phenyl}-1,3-thiazol-4-yl)piperidin-1-yl]-2-oxoethyl}-3H-imidazo[4,5-b]pyridine

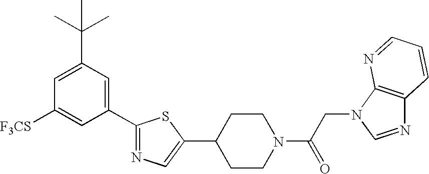

Using the method of Example 1, Step L with the products of Example 1, Step K and Example 11, Step L as starting materials the title compound was obtained. ¹H NMR (CD₃OD): δ 8.39 (dd, 1H, J=4.8, 1.1 Hz), 8.38 (s, 1H), 8.15 (t, 1H, J=1.6 Hz), 8.12 (dd, 1H, J=8.0, 1.3 Hz), 8.07 (s, 1H), 7.80 (s, 1H), 7.37 (dd, 1H, J=8.0, 4.8 Hz), 7.32 (s, 1H), 5.45 (½AB, 1H, J=17.0 Hz), 5.39 (½AB, 1H, J=16.9 Hz), 4.58 (bd, 1H, J=13.3 Hz), 4.23 (bd, 1H, J=13.5 Hz), 3.46 (dt, 1H, Jₜ=12.4, J_{d}=2.5 Hz), 3.21 (tt, 1H, J=11.5, 3.6 Hz), 2.95 (dt, 1H, Jₜ=12.8, J_{d}=2.5 Hz), 2.28 (bd, 1H, J=12.0 Hz), 2.15 (bd, 1H, J=12.5 Hz), 1.94 (dquart, 1H, J_{q}=12.6, J_{d}=3.6 Hz), 1.79 (dquart, 1H, J_{q}=12.6, J_{d}=3.6 Hz), 1.42 (s, 18H). LRMS calc: 559.2 obs: 560.3 (M+H).

### Example 12

### Step A 4-methoxy-1H-imidazole

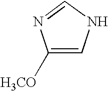

A solution of potassium carbonate (7.5 g, 54.3 mmol) in water (20 mL) was treated with aminoacetonitrile hydrochloride (9.0 g, 97.3 mmol) in portions with stirring. After effervescence stopped, the solution was extracted in a liq/liq continuous extractor for 2 h (EtOAc). The organic was dried over MgSO₄, filtered and concentrated to a residue (2.70 g). Sodium sulfate (7.0 g; 50 mmol) was placed in a 3-neck flask followed by trimethylorthoformate (70 mL) and con. H₂SO₄ (2 drops). A variable take-off distillation head was attached and the mixture heated to reflux. Aminoacetonitrile (2.7 g, 50 mmol) was added dropwise via syringe over 5 min. Methanol was distilled at a reflux ratio of 10:1 for 20 min. The solution was cooled, filtered and concentrated to a residue (4.9 g). The crude methyl (cyanomethyl)imidoformate was dissolved in methanolic NaOCH₃ (50 mL; 1M). The solution was refluxed for 2 h. The now black solution was neutralized with con HCl (4 mL). The mixture was filtered and evaporated to a residue which was adsorbed onto silica gel (CH₂Cl₂). The silica gel was eluted (50:50:1 CH₂C₁₂/acetone/triethylamine) without fractionation to give the title compound (1.16 g).

### Step B (4-methoxy-1H-imidazol-1-yl)acetic acid methyl ester

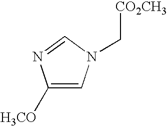

Using the method of Example 7, Step B and the product of Example 12, Step A the title compound was obtained.

### Step C (4-methoxy-1H-imidazol-1-yl)acetic acid hydrochloride

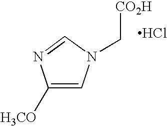

Using the method of Example 7, Step D and the product of Example 12, Step B the title compound was obtained.

### Step D 4-[2-(3,5-di-tert-butyl-4-methoxyphenyl)-1,3-thiazol-4-yl]-1H-[(4-methoxy-1-imidazol-1-yl)acetyl]piperidine

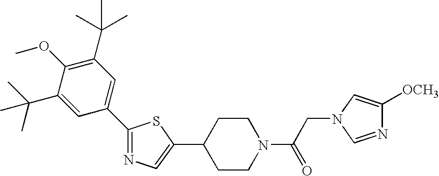

Using the method of Example 1, Step L with the products of Example 1, Step H and Example 12, Step C as starting materials the title compound was obtained. ¹H NMR (CD₃OD): δ 7.80 (s, 2H), 7.26 (s, 1H), 7.13 (s, 1H), 6.41 (s, 1H), 5.01 (½AB, 1H, J=16.7 Hz), 4.95 (½AB, 1H, J=16.7 Hz), 4.59 (bd, 1H, J=12.8 Hz), 4.09 (bdd, 1H, J=13.0, 9.3 Hz), 3.73 (s, 3H), 3.71 (s, 3H), 3.30 (bt, 1H, J=12.0 Hz), 3.11 (dt, 1H, Jₜ=11.8, J_{d}=3.6 Hz), 2.87 (bt, 1H, J=12.9 Hz), 2.17 (bd, 1H, J=13.2 Hz), 2.11 (bd, 1H, J=12.5 Hz), 1.79 (dquart, 1H, J_{q}=12.5, J_{d}=3.4 Hz), 1.70 (dquart, 1H, J_{q}=12.7, J_{d}=4.2 Hz), 1.46 (s, 18H). LRMS 524.3 obs: 525.3 (M+H).

### Example 13

### Step A 3-tert-butyl-4-hydroxybenzoic acid methyl ester

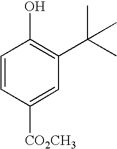

A solution of 3-tert-butyl-4-hydroxybenzoic acid (10.0 g; 51.5 mmol) in methanol (100 mL) was treated with con. H₂SO₄ (1 mL) and refluxed for 1 h. The reaction was partitioned between water (150 mL) and isopropyl acetate (200 mL). The organic was dried over MgSO₄, filtered and concentrated to afford the title compound (10.7 g).

### Step B 3-tert-butyl-4[(3-methylbut-2-en-1-yl)oxy]benzoic acid methyl ester

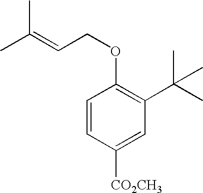

A solution of 3-tert-butyl-4-hydroxybenzoic acid methyl ester (10.7 g; 51.3 mmol) and 1-bromo-3-methylbut-2-ene (6.00 mL; 51.3 mmol) in DMF (150 mL) was treated with Cs₂CO₃ (30.10 g; 92.3 mmol). The mixture was stirred for 48 h at ambient temperature. The reaction was partitioned between pH4 phthalate buffer solution (200 mL) and isopropyl acetate (200 mL). The organic was washed with water (2×150 mL), dried over MgSO₄, filtered and concentrated to a residue. A portion of the crude (5 g) was chromatographed on SiO₂ (1:1 hexane/CH₂Cl₂). The derived material was flushed with xylene (3×10 mL) affording the title compound (1.38 g).

### Step C 8-tert-butyl-4,4-dimethylchromane-6-carboxylic acid methyl ester

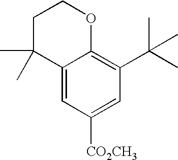

A −78° solution of 3-tert-butyl-4[(3-methylbut-2-en-1-yl)oxy]benzoic acid methyl ester (1.2 g; 4.3 mmol) in dry CH₂Cl₂ (20 mL) was treated with trifluoromethanesulfonic acid (0.2 mL). The reaction was warmed to ambient temperature and stirred 1 h. The reaction was partitioned between pH7 phosphate buffer solution (30 mL) and isopropyl acetate (30 mL). The organic was dried over MgSO₄, filtered and concentrated to a residue. The residue was chromatographed on SiO₂ (4:1 hexane/CH₂Cl₂) giving the title compound (475 mg).

### Step D 8-tert-butyl-4,4-dimethylchromane-6-carboxylic acid

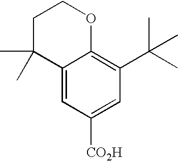

A solution of 8-tert-butyl-4,4-dimethylchromane-6-carboxylic acid methyl ester (475 mg, 1.70 mmol) in methanol (5 mL) was treated with aq NaOH (2.5N; 1 mL). THF (1.5 mL) was added and the reaction was refluxed 2 h. The reaction was partitioned between 0.1N HCl (50 mL) and isopropyl acetate (100 mL). The organic was dried over MgSO₄, filtered and evaporated to afford the title compound (438 mg).

### Step E 8-tert-butyl-4,4-dimethylchromane-6-carbonyl chloride

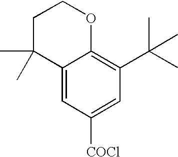

Using the method of Example 1, Step A and the product of Example 13, Step D (309 mg; 1.20 mmol) as starting material the title compound was obtained.

### Step F 8-tert-butyl-4,4-dimethylchromane-6-carboxamide

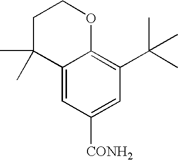

A solution of 8-tert-butyl-4,4-dimethylchromane-6-carbonyl chloride (approx. 1.20 mmol) in dioxane (5 mL) was added to a solution of ammonia in dioxane (3.5 mL; 0.5M) at ambient temperature. The resultant mixture was partitioned between water (10 mL) and isopropyl acetate (10 mL). The organic was dried over MgSO₄, filtered and concentrated to a residue affording the title compound (307 mg).

### Step G 8-tert-butyl-4,4-dimethylchromane-6-carboxthioamide

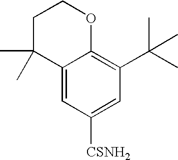

Using the method of Example 1, Step F with the product of Example 13, Step F as starting material the title compound was obtained.

### Step H 9H-fluoren-9-ylmethyl4-[2-(8-tert-butyl-4,4-dimethyl-3,4-dihydro-2H-chromen-6-yl)-1,3-thiazol-4-yl]piperidine-1-carboxylate

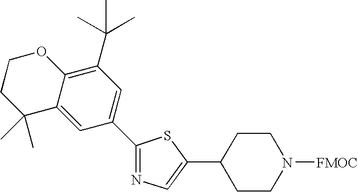

Using the method of Example 1, Step G with the products of Example 1, Step C and Example 13, Step G as starting materials the title compound was obtained.

### Step J 4-[2-(8-tert-butyl-4,4-dimethyl-3,4-dihydro-2H-chromen-6-yl)-1,3-thiazol-4-yl]piperidine

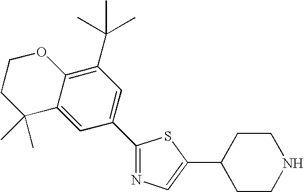

Using the method of Example 1, Step H with the product of Example 13, Step H as starting material the title compound was obtained.

### Step K 4-[2-(8-tert-butyl-4,4-dimethyl-3,4-dihydro-2H-chromen-6-yl)-1,3-thiazol-4-yl]-1-[(2,4-dimethyl-1H-imidazol-1-yl)acetyl]piperidine

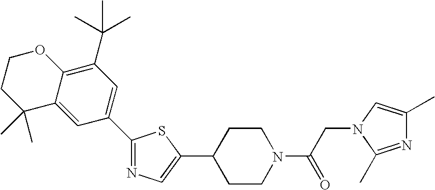

Using the method of Example 1, Step L with the products of Example 13, Step J and Example 2, Step B as starting materials the title compound was obtained. 1H NMR (CDCl₃): δ 7.63 (s, 1H), 7.52 (s, 1H), 6.78 (s, 1H), 6.59 (bs, 1H), 4.72 (vbs, 2H), 4.64 (bd, 1H, J=13.0 Hz), 3.87 (bd, 1H, J=13.3 Hz), 3.72-3.60 (bmult, 2H), 3.29 (bt, 1H, J=12.4 Hz), 3.09 (vbt, 1H, J=10.6 Hz), 2.87-2.84 (bmult, 2H), 2.40 (bs, 3H), 230-2.22 (bs overlapping bd, 4H total), 2.16 (bd, 1H, J=10.5 Hz), 1.83 (bt, 1H, J=6.8 Hz), 1.74 (vbmult, 2H), 1.41 (s, 9H), 1.38 (s, 6H). LRMS calc: 520.3 obs: 521.4 (M+H).

### Example 14

### Step A 4-chloro-2-methyl-1H-imidazole

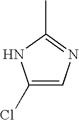

A solution of 2-methyl-1H-imidazole (1.05 g, 12.84 mmol) in acetonitrile (35 mL) was treated with N-chlorosuccinimide (1.71 g, 12.84 mmol). The reaction was stirred 8 h and then evaporated. The crude product was digested in CH₂Cl₂ (20 mL) and the solution evaporated to a brown residue. The residue was adsorbed onto silica gel and eluted (100:2.5:1 CH₂Cl₂/MeOH/triethylamine) without fractionation. The eluant was concentrated and the residue chromatographed on silica gel (preparative TLC; 100:5:1 CH₂Cl₂/methanol/triethylamine). Part of the derived mixture of mono and dichloroimidazoles was rechromatographed on Chiralcel AS stationary phase (5% ethanol/heptane; λ=220 nM) to afford the less mobile title compound (109 mg).

### Step B (4-chloro-2-methyl-1H-imidazol-1-yl)acetic acid tert-butyl ester

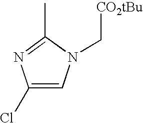

Using the method of Example 2, Step A and the product of Example 14, Step A the title compound was obtained.

### Step C (4-chloro-2-methyl-1H-imidazol-1-yl)acetic acid hydrotrifluoroacetate

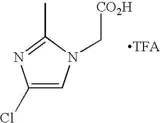

Using the method of Example 2, Step B and the product of Example 14, Step B as starting material the title compound was obtained.

### Step D 1-[(4-chloro-2-methyl-1H-imidazol-1-yl)acetyl]-4-[2-(3,5-di-tert-butyl-4-methoxyphenyl)-1,3-thiazol-4-yl]piperidine

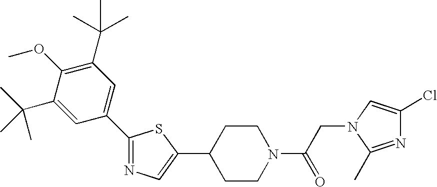

Using the method of Example 1, Step L with the products of Example 1, Step H and Example 14, Step C as the starting materials the title compound was obtained. ¹H NMR (CDCl₃): δ 7.81 (s, 2H), 6.87 (s, 1H), 6.75 (s, 1H), 4.70-4.67 (bs overlapping bd, 3H), 3.87 (bd, 1H, J=13.7 Hz), 3.74 (s, 3H), 3.34 (bt, 1H, J=11.9 Hz), 3.12 (tt, 1H, J=11.4, 3.4 Hz), 2.90 (bt, 1H, J=11.7 Hz), 2.34 (s, 3H), 2.30 (bd, 1H, J=11.7 Hz), 2.20 (bd, 1H, J=12.5 Hz), 1.78 (bdpent, 2H, Jₚ=12.5, J_{d}=3.3 Hz), 1.50 (s, 18H). LRMS calc: 542.3 obs: 543.3 (M+H).

### Step E 4-chloro-2,5-dimethyl-1H-imidazole

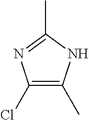

Using the method of Example 14, Step A and 2,4-dimethylimidazole as starting material the title compound was obtained.

### Step F (4-chloro-2,5-dimethyl-1H-imidazol-1-yl)acetic acid tert-butyl ester

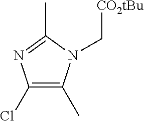

Using the method of Example 2, Step A and the product of Example 14, Step E the title compound was obtained.

Step G (4-chloro-2,5-dimethyl-1H-imidazol-1-yl)acetic acid hydrotrifluoroacetate

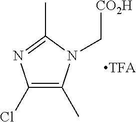

Using the method of Example 2, Step B and the product of Example 14, Step F as starting material the title compound was obtained.

### Step H 1-[(4-chloro-2,5-dimethyl-1H-imidazol-1-yl)acetyl]-4-[2-(3,5-di-tert-butyl-4-methoxyphenyl)-1,3-thiazol-4-yl]piperidine

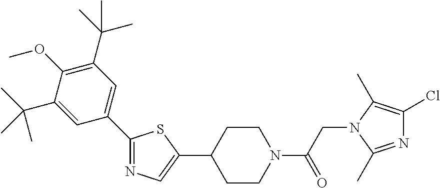

Using the method of Example 1, Step L with the products of Example 1, Step H and Example 14, Step G as the starting materials the title compound was obtained. ¹H NMR (CDCl₃): δ 7.79 (s, 2H), 6.85 (s, 1H), 4.65 (bd, 1H, J=13.2 Hz), 4.59 (½AB, 1H, J=16.7 Hz), 4.55 (½AB, 1H, J=16.7 Hz), 3.91 (bd, 1H, J=13.4 Hz), 3.71 (s, 3H), 3.33 (bt, 1H, J=12.1 Hz), 3.13-3.06 (mult, 2H), 2.87 (bt, 1H, J=12.0 Hz), 2.31-228 (s overlapping with bd, 4H total), 2.17 (bd, 1H, J=13.3 Hz), 2.09 (s, 3H), 1.76 (bquart, 2H, J=11.1 Hz), 1.47 (s, 18H). LRMS calc: 556.3 obs: 557.4 (M+H).

### Example 15

### Step A 3,5-di-tert-butylbenzoyl chloride

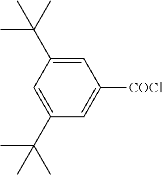

Using the method of Example 1, Step A and 3,5-di-tert-butylbenzoic acid as the starting material the title compound was obtained.

### Step B 3,5-di-tert-butylbenzamide

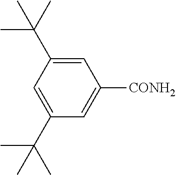

Using the method of Example 13, Step F with the product of Example 15, Step A as the starting material the title compound was obtained.

### Step C 3,5-di-tert-butylbenzthioamide

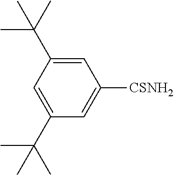

Using the method of Example 1, Step F with the product of Example 15, Step B as the starting material the title compound was obtained.

### Step D 9H-fluoren-9-ylmethyl-4-[2-(3,5-di-tert-butylphenyl)-1,3-thiazol-4-yl]piperidine-1-carboxylate

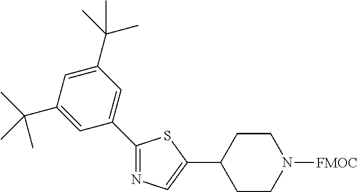

Using the method of Example 1, Step G with the products of Example 1, Step C and Example 15, Step C as the starting materials the title compound was obtained.

### Step E 4-[2-(3,5-di-tert-butylphenyl)-1,3-thiazol-4-yl]piperidine

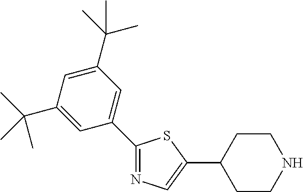

Using the method of Example 1, Step H with the product of Example 15, Step D as the starting material the title compound was obtained.

### Step F 1-(chloroacetyl)-4-[2-(3,5-di-tert-butylphenyl)-1,3-thiazol-4-yl]piperidine

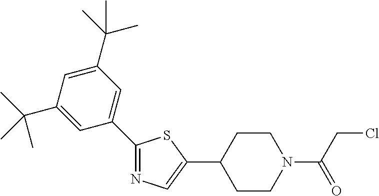

A −10° solution of the product of Example 15, Step E (29 mg; 0.08 mmol) in dry THF (1 mL) was treated with triethylamine (14 μL; 0.10 mmol) and chloroacetyl chloride (7 μL; 0.08 mmol). The mixture was stirred at ambient temperature for 1 h, then filtered. The filtrate containing the title compound was used directly in the next reaction.

### Step G 3-(2-{4-[2-(3,5-di-tert-butyl phenyl)-1,3-thiazol-4-yl]piperidin-1-yl}-2-oxoethyl)-3H-imidazo[4,5-c]pyridine and 1-(2-{4-[2-(3,5-di-tert-butylphenyl)-1,3-thiazol-4-yl]piperidin-1-yl}-2-oxoethyl)-1H-imidazo[4,5-c]pyridine

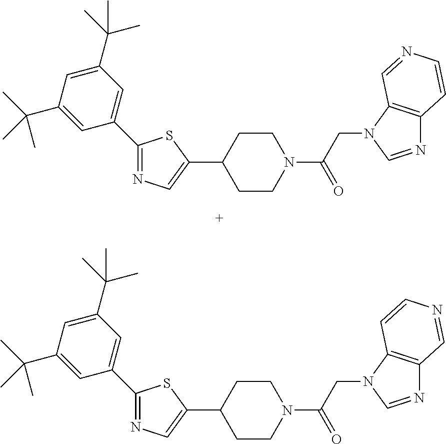

A THF solution of the product of Example 15, Step F was added to a solution of 5-azabenzimidazole (48 mg; 0.405 mmol) in dry DMF (1 mL). Cesium carbonate (132 mg; 0.405 mmol) was added and the mixture stirred for 2 h. The reaction was diluted with isopropyl acetate (10 mL) and washed with pH7 phosphate buffer solution (3×10 mL). The organic was dried over MgSO₄, filtered and evaporated to a residue. Chromatography over silica gel (prep. TLC; 10:1 CH₂Cl₂/MeOH) afforded the more mobile 3-(3H) title compound and the less mobile 1-(1B) title compound. ¹H NMR (CD₃OD): δ 8.88 (s, 1H), 8.37 (s, 1H), 8.36 (d, 1H, J=4.0 Hz), 7.76 (d, 2H, J=1.8 Hz), 7.73 (d, 1H, J=5.8 Hz), 7.57 (t, 1H, J=1.7 Hz), 7.21 (s, 1H), 5.55 (½AB, 1H, J=17.2 Hz), 5.49 (½AB, 1H, J=17.1 Hz), 4.58 (bd, 1H, J=13.3 Hz), 4.16 (bd, 1H, J=13.5 Hz), 3.43 (dt, 1H, Jₜ=13.6, J_{d}=2.3 Hz), 3.18 (tt, 1H, J=11.8, 3.4 Hz), 2.93 (dt, 1H, Jₜ=12.9, J_{d}=2.3 Hz), 2.26 (bd, 1H, J=13.3 Hz), 2.14 (bd, 1H, J=13.2 Hz), 1.95 (dquart, 1H, J_{q}=12.7, J_{d}=4.0 Hz), 1.77 (dquart, 1H, J_{q}=12.5, J_{d}=4.0 Hz), 1.37 (s, 18H). LRMS calc: 515.3 obs: 516.4. ¹H NMR (CD₃OD): δ 8.94 (s, 1H), 8.36 (d, 1H, J=5.8 Hz), 8.31 (s, 1H), 7.76 (d, 2H, J=1.7 Hz), 7.64 (d, 1H, J=5.8 Hz), 7.57 (t, 1H, J=1.7 Hz), 7.21 (s, 1H), 5.48 (½AB, 1H, J=17.3 Hz), 5.42 (½AB, 1H, J=17.2 Hz), 4.57 (bd, 1H, J=13.3 Hz), 4.16 (bd, 1H, J=13.5 Hz), 3.43 (dt, 1H, Jₜ=14.0, J_{d}=2.5 Hz), 3.18 (tt, 1H, J=11.5, 3.3 Hz), 2.93 (bt, 1H, J=12.7 Hz), 2.26 (bd, 1H, J=13.2 Hz), 2.14 (bd, 1H, J=13.2 Hz), 1.95 (dquart, 1H, J_{q}=12.6, J_{d}=3.6 Hz), 1.77 (dquart, 1H, J_{q}=12.8, J_{d}=3.8 Hz), 1.37 (s, 18H). LRMS calc: 515.3 obs: 516.4 (M+H).

### Example 16

### Step A 3-(2-{4-[2-(3,5-di-tert-butylphenyl)-1,3-thiazol-4-yl]piperidin-1-yl}-2-oxoethyl)-3H-imidazo[4,5-b]pyridine and 1-(2-{4-[2-(3,5-di-tert-butylphenyl)-1,3-thiazol-4-yl]piperidin-1-yl}-2-oxoethyl)-1H-imidazo[4,5-b]pyridine

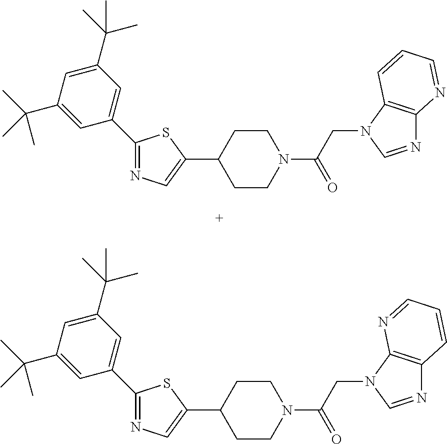

A THF solution of the product of Example 15, Step F was added to a solution of 4-azabenzimidazole (774 mg; 6.50 mmol) in dry DMF (6 mL). Cesium carbonate (2.12 g; 6.50 mmol) was added and the mixture stirred for 2 h. The reaction was diluted with isopropyl acetate (50 mL) and washed with brine (3×15 mL). The organic was dried over MgSO₄, filtered and evaporated to a residue. Chromatography over silica gel (prep. TLC; 15:1 CH₂Cl₂/MeOH) afforded the more mobile 3-(3H) and the less mobile 1-(1H) title compounds (20 mg and 80 mg respectively). 1H NMR (CD₃OD): δ 8.46 (dd, 1H, J=4.8, 1.1 Hz), 8.40 (s, 1H), 8.03 (dd, 1H, J=8.2, 1.3 Hz), 7.78 (d, 2H, J=1.6 Hz), 7.59 (t, 1H, J=1.6 Hz), 7.37 (dd, 1H, J=8.2, 4.8 Hz), 7.22 (s, 1H), 5.49 (½AB, 1H, J=17.2 Hz), 5.43 (½AB, 1H, J=17.1 Hz), 4.59 (bd, 1H, J=13.0 Hz), 4.18 (bd, 1H, J=13.7 Hz), 3.44 (dt, 1H, Jₜ=13.3, J_{d}=2.5 Hz), 3.19 (tt, 1H, J=11.9, 3.7 Hz), 2.94 (bt, 1H, J=13.1 Hz), 2.26 (bd, 1H, J=12.8 Hz), 2.15 (bd, 1H, J=12.6 Hz), 1.96 (dquart, 1H, J_{q}=12.5, J_{d}=3.8 Hz), 1.77 (dquart, 1H, J_{q}=12.6, J_{d}=3.8 Hz), 1.39 (s, 18H). LRMS calc: 515.3 obs: 516.4 (M+H). ¹H NMR (CD₃OD): δ 8.39 (dd, 1H, J=5.1, 1.4 Hz), 8.38 (s, 1H), 8.12 (dd, 1H, J=8.2, 1.4 Hz), 7.78 (d, 2H, J=1.8 Hz), 7.58 (t, 1H, J=1.8 Hz), 7.37 (dd, 1H, J=8.1, 4.9 Hz), 7.22 (s, 1H), 5.44 (½AB, 1H, J=16.9 Hz), 5.38 (½AB, 1H, J=16.9 Hz), 4.58 (bd, 1H, J=13.3 Hz), 4.23 (bd, 1H, J=13.5 Hz), 3.45 (dt, 1H, Jₜ=13.0, J_{d}=2.5 Hz), 3.19 (tt, 1H, J=11.4, 3.7 Hz), 2.93 (dt, 1H, Jₜ=12.8, J_{d}=2.5 Hz), 2.28 (bd, 1H, J=13.0 Hz), 2.14 (bd, 1H, J=12.8 Hz), 1.98 (dquart, 1H, J_{q}=12.5, J_{d}=4.1 Hz), 1.77 (dquart, 1H, J_{q}=12.6, J_{d}=4.01 Hz), 1.39 (s, 18H). LRMS calc: 515.3 obs: 516.3 (M+H).

### Example 17

### Step A N-Ethylaminoacetone hydrochloride

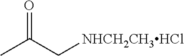

Using the method of Example 5, Step A with N-ethyl benzyl amine in place of N-methyl benzyl amine as starting material the title compound was obtained.

### Step B (3-ethyl-5-methyl-2-oxo-2,3-dihydro-1H-imidazol-1-yl)acetic acid ethyl ester

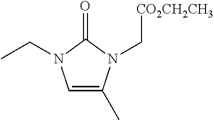

A solution of N-ethylaminoacetone hydrochloride (503 mg; 3.66 mmol) in dry DMF (10 mL) was treated with ethyl isocyanatoacetate (472 mg; 3.66 mmol) followed by cesium carbonate (1.19 g; 3.66 mmol). The mixture was stirred at ambient temperature for 16 h. The mixture was filtered and the filtrate flushed with xylene (3×30 mL; 1 mm vacuum; 60° bath). The derived residue was dissolved in dry benzene (20 mL) and treated with TFA (4 mL). The solution was refluxed 30 min. The reaction was evaporated and flushed with toluene (2×30 mL). The crude product was adsorbed onto silica gel and eluted (MTBE) without fractionation. The eluant was evaporated to a residue and chromatographed over silica gel (preparative TLC, MTBE) to give the title compound (429 mg).

### Step C Potassium (3-ethyl-5-methyl-2-oxo-2,3-dihydro-1H-imidazol-1-yl)acetate

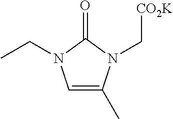

Using the method of Example 8, Step B with the product of Example 17, Step B as starting material the title compound was obtained.

### Step D 3-(2-{4-[2-(3,5-di-tert-butyl-4-methoxyphenyl)-1,3-thiazol-4-yl]piperidin-1-yl}-2-oxoethyl)-1-ethyl-4-methyl-1,3-dihydro-2H-imidazol-2-one

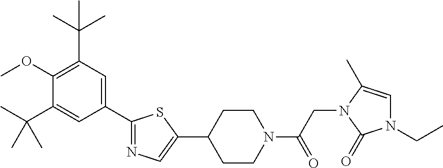

Using the method of Example 1, Step L with the products of Example I, Step H and Example 17, Step C as the starting materials the title compound was obtained. ¹H NMR (CD₃OD): δ 7.84 (s, 2H), 7.18 (s, 1H), 6.26 (s, 1H), 4.69 (½AB, 1H, J=17.2 Hz), 4.61 (½AB, 1H, J=17.1 Hz), 4.60 (bd, 1H, J=13.0 Hz), 4.15 (bd, 1H, J=13.5 Hz), 3.75 (s, 3H), 3.66 (quart, 2H, J=7.3 Hz), 3.36 (bt, 1H, J=12.0 Hz), 3.15 (tt, 1H, J=11.7, 3.2 Hz), 2.91 (bt, 1H, J=11.8 Hz), 2.22 (bd, 1H, J=12.5 Hz), 2.15 (bd, 1H, J=12.7 Hz), 1.85 (dquart, 1H, J_{q}=12.3, J_{d}=3.6 Hz), 1.73 (dquart, 1 H, J_{q}=12.5, J_{d}=3.4 Hz), 1.50 (s, 18H), L27 (t, 3H, J=7.2 Hz). LRMS calc: 552.3 obs: 553.3 (M+H).

### Example 18

### Step A Aminoacetone hydrochloride

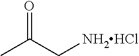

A solution of chloroacetone (800 μL; 10.05 mmol) in dry d⁶-deuteroacetone (10 mL) was treated with sodium azide (790 mg; 12.14 mmol). The mixture was stirred for 18 h (progress followed by NMR). The mixture was filtered and concentrated to a residue (850 mg). The residue was dissolved in ethanol (10 mL) and treated with con. HCl (2 mL). The solution was combined with 10% Pd/C hydrogenation catalyst (175 mg) and shaken under a hydrogen atmosphere (50 psi) for 2 h. The mixture was filtered through Celite and evaporated to give the title compound (707 mg).

### Step B 1-ethyl-5-methyl-1,3-dihydro-2H-imidazol-2-one

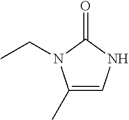

Using the method of Example 17, Step B with the product of Example 18, Step A and ethyl isocyanate as the starting materials the title compound was obtained.

### Step C (3-ethyl-4-methyl-2-oxo-2,3-dihydro-1H-imidazol-1-yl)acetic acid tert-butyl ester

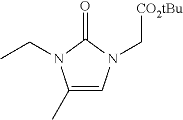

A solution of 1-ethyl-5-methyl-1,3-dihydro-2H-imidazol-2-one (250 mg; 1.98 mmol) in dry DMF (6 mL) was treated with tert-butyl bromoacetate (289 μL; 1.98 mmol). Sodium hydride (59 mg; 80%; 1.98 mmol) was added and the mixture stirred at ambient temperature for 1 h. The reaction mixture was diluted with isopropyl acetate (30 mL) and washed with pH7 phosphate buffer (3×10 mL). The organic was dried over MgSO₄, filtered and evaporated to a residue. The crude product was chromatographed over silica gel (preparative TLC; 30:1 CH₂Cl₂/MeOH) to give the title compound (172 mg).

### Step D (3-ethyl-4-methyl-2-oxo-2,3-dihydro-1H-imidazol-1-yl)acetic acid

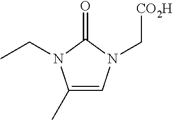

Using the method of Example 2, Step 13 and the product of Example 18, Step C as starting material the title compound was obtained.

### Step E 1-(2-{4-[2-(3,5-di-tert-butyl-4-methoxyphenyl)-1,3-thiazol-4-yl]piperidin-1-yl}-2-oxoethyl)-3-ethyl-4-methyl-1,3-dihydro-2H-imidazol-2-one

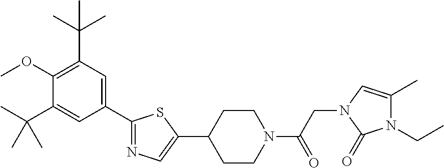

Using the method of Example 1, Step L with the products of Example 1, Step H and Example 18, Step D the title compound was obtained. ¹H NMR (CD₃OD): δ 7.82 (s, 211), 7.15 (s, 1H), 6.18 (d, 1H, J=1.4 Hz), 4.62 (½AB, 1H, J=16.7 Hz), 4.60 (bd, 1H, J=13.0 Hz), 4.52 (½AB, 1H, J=16.8 Hz), 4.06 (bd, 1H, J=13.5 Hz), 3.73 (s, 3H), 3.70 (quart, 2H, J=7.3 Hz), 3.30 (bt, 1 H, J=11.4 Hz), 3.12 (tt, 1H, J=11.5, 3.4 Hz), 2.87 (dt, 1H, Jₜ=11.8, J_{d}=2.7 Hz), 2.19 (bd, 1H, J=13.3 Hz), 2.14-2.11 (bd overlapping d, 4H total, J_{d}=1.3 Hz), 1.81 (dquart, 1H, J_{q}=12.6, J_{d}=3.7 Hz), 1.70 (dquart, 1H, J_{q}=12.5, J_{d}=3.8 Hz), 1.48 (s, 18H), 1.23 (t, 3H, J=7.2 Hz). LRMS calc: 552.3 obs: 553.4 (M+H).

### Step F 1-(2-{4-[2-(3,5-di-tert-butylphenyl)-1,3-thiazol-4-yl]piperidin-1-yl}-2-oxoethyl)-3-ethyl-4-methyl-1,3-dihydro-2H-imidazol-2-one

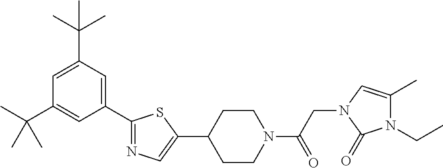

Using the method of Example 1, Step L with the products of Example 15, Step E and Example 18, Step D as the starting materials the title compound was obtained. ¹H NMR (CD₃OD): δ 7.75 (d, 2H, J=1.6 Hz), 7.55 (t, 1H, J=1.7 Hz), 7.17 (s, 1H), 6.16 (s, 1H), 4.59 (½AB overlapping bd, 2H total, J_{AB}=16.7 Hz), 4.51 (½AB, 1H, J=16.6 Hz), 4.05 (bd, 1H, J=13.1 Hz), 3.69 (quart, 2H, J=7.2 Hz), 3.31 (bt, 1H, J=12.0 Hz), 3.12 (tt, 1H, J=11.7, 3.5 Hz), 2.86 (bt, 1H, J=12.8 Hz), 2.17 (bd, 1H, J=12.9 Hz), 2.12-2.09 (s overlapping bd, 4H total), 1.80 (dquart, 1H, J_{q}=12.2, J_{d}=3.8 Hz), 1.70 (dquart, 1H, J_{q}=12.3, J_{d}=3.8 Hz), 1.36 (s, 18H), 1.21 (t, 3H, J=7.3 Hz). LRMS calc: 522.3 obs: 524.4 (M+H).

### Example 19

### Step A (2-oxo-23-dihydro-1H-benzimidazol-1-yl)acetic acid tert-butyl ester

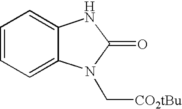

Using the method of Example 18, Step C and 1,3-dihydro-2H-benzimidazol-2-one as starting material the title compound was obtained.

### Step B (2-oxo-2,3-dihydro-1H-benzimidazol-1-yl)acetic acid

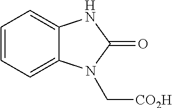

Using the method of Example 2, Step B with the product of Example 19, Step A as starting material the title compound was obtained.

### Step C 1-(2-{4-[2-(3,5-di-tert-butyl-4-methoxyphenyl)-1,3-thiazol-4-yl]piperidin-1-yl}-2-oxoethyl)-1,3-dihydro-2H-benzimidazol-2-one

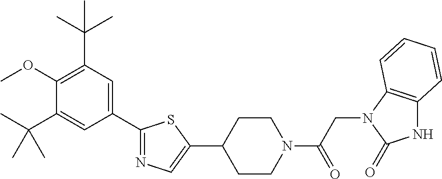

Using the method of Example 1, Step L with the products of Example 1, Step H and Example 19, Step B as the starting materials the title compound was obtained. ¹H NMR (CD₃OD): δ 7.83 (s, 2H), 7.15 (s, 1H), 7.07 (bmult, 4H), 4.89 (½AB, 1H, J=17.1 Hz), 4.81 (½AB, 1H, J=17.0 Hz), 4.58 (bd, 1H, J=13.5 Hz), 4.19 (bd, 1H, J=13.4 Hz), 3.73 (s, 3H), 3.38 (bt, 1H, J=12.3 Hz), 3.14 (tt, 1H, J=11.7, 3.7 Hz), 2.89 (bt, 1H, J=12.3 Hz), 2.22 (bd, 1H, J=13.2 Hz), 2.12 (bd, 1H, J=13.3 Hz), 1.85 (dquart, 1H, J_{q}=12.5, J_{d}=3.7 Hz), 1.70 (dquart, 1H, J_{q}=12.6, J_{d}=3.6 Hz), 1.48 (s, 18H). LRMS calc: 560.3 obs: 561.4 (M+H).

### Step D (3-methyl-2-oxo-2,3-dihydro-1H-benzimidazol-1-yl)acetic acid tert-butyl ester

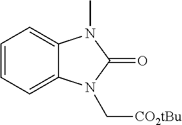

Using the method of Example 18, Step C with the product of Example 19, Step A and methyl iodide as the starting materials the title compound was obtained.

### Step E (3-methyl-2-oxo-2,3-dihydro-1H-benzimidazol-1-yl)acetic acid

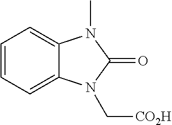

Using the method of Example 2, Step B with the product of Example 19, Step D as starting material the title compound was obtained.

### Step F 1-(2-{4-[2-(3,5-di-tert-butyl-4-methoxyphenyl)-1,3-thiazol-4-yl]piperidin-1-yl}-2-oxoethyl)-3-methyl-1,3-dihydro-2H-benzimidazol-2-one

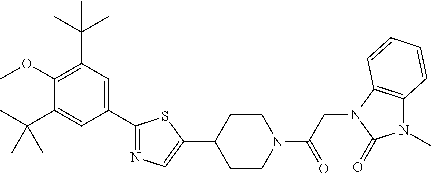

Using the method of Example 1, Step L with the products of Example 1, Step H and Example 19, Step E as the starting materials the title compound was obtained. ¹H NMR (CD₃OD): δ 7.81 (s, 2H), 7.15-7.07 (mult, 5H), 4.92 (½AB, 1H, J=17.2 Hz), 4.85 (½AB, 1H, J=17.0 Hz), 4.56 (vbs, 1H), 4.19 (bd, 1H, J=13.0 Hz), 3.72 (s, 3H), 3.44 (s, 3H), 3.38 (bt, 1H, J=13.1 Hz), 3.14 (tt, 1H, J=11.5, 3.9 Hz), 2.89 (dt, 1H, Jₜ=12.5, J_{d}=2.2 Hz), 2.22 (bd, 1H, J=13.2 Hz), 2.12 (bd, 1H, J=13.3 Hz), 1.86 (dquart, 1H, J_{q}=12.5, J_{d}=3.7 Hz), 1.70 (dquart, 1H, J_{q}=12.6, J_{d}=3.6 Hz), 1.47 (s, 18H). LRMS calc: 574.3 obs: 575.4 (M+H).

### Example 20

### Step A 2-ethyl-4-methyl-1H-imidazol-1-ylacetic acid tert-butyl ester

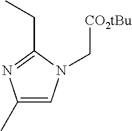

Using the method of Example 2, Step A and 2-ethyl-4-methylimidazole as starting material the title compound was obtained. The title compound was purified by silica gel chromatography (preparative TLC; 100:2.5:1 CH₂C₂/MeOH/Et₃N).

### Step B 2-ethyl-4-methyl-1H-imidazol-1-ylacetic acid hydrotrifluoroacetate

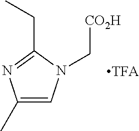

Using the method of Example 2, Step B and the product of Example 20, Step A as starting material the title compound was obtained.

### Step C 4-[2-(3,5-di-tert-butyl-4-methoxyphenyl)-1,3-thiazol-4-yl]-1-[(2-ethyl-4-methyl-1H-imidazol-1-yl)acetyl]piperidine

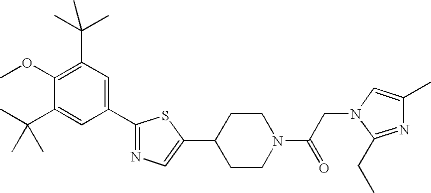

Using the method of Example 1, Step L and starting with the products of Example 1, Step H and Example 20, Step B the title compound was obtained. 1H NMR (CDCl₃): δ 7.81 (s, 2H), 6.86 (s, 1H), 6.56 (bs, 1H), 4.74-4.56 (bmult, 3H), 3.89 (bd, 1H, J=12.6 Hz), 3.73 (s, 3H), 3.31 (bt, 1H, J=12.8 Hz), 3.11 (vbt, 1H, J=10.0 Hz), 2.87 (bt, 1H, J=11.6 Hz), 2.64 (quart, 2H, J=7.5 Hz), 2.33-2.15 (s overlapping bmult, 5H), 1.82-1.62 (bmult, 2H), 1.49 (s, 18H), 1.35 (t, 3H, J=7.6 Hz). LRMS calc: 536.3 obs: 537.4 (M+H).

### Example 21

### Step A 3,5-di-tert-butyl-4-hydroxybenzoyl chloride

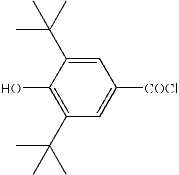

Using the method of Example 1, Step A and 3,5-di-tert-butyl-4-hydroxybenzoic acid as the starting material the title compound was obtained.

### Step B 3,5-di-tert-butyl-4-hydroxybenzamide

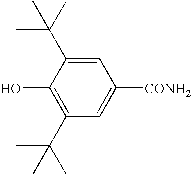

Using the method of Example 13, Step F with the product of Example 21, Step A as the starting material the title compound was obtained.

### Step C 3,5-di-tert-butyl-4-hydroxybenzthioamide

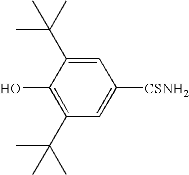

Using the method of Example 1, Step F with the product of Example 21, Step B as the starting material the title compound was obtained.

### Step D 9H-fluoren-9-ylmethyl-4-[2-(3,5-di-tert-butyl-4-hydroxyphenyl)-1,3-thiazol-4-yl]piperidine-1-carboxylate

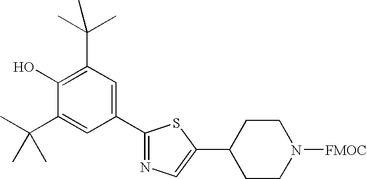

Using the method of Example 1, Step G with the products of Example 1, Step C and Example 21, Step C as the starting materials the title compound was obtained.

### Step E 2,6-di-tert-butyl-4-(4-piperidin-4-yl-1,3-thiazol-2-yl)phenol

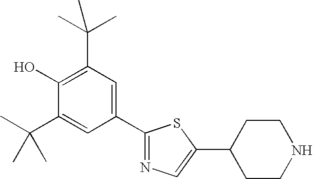

Using the method of Example 1, Step H with the product of Example 21, Step D as the starting material the title compound was obtained.

### Step F 2,6-di-tert-butyl-4-{4-[1-(chloroacetyl)piperidin-4-yl]-1,3-thiazol-2-yl}phenol

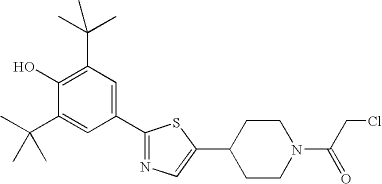

Using the method of Example 15, Step F with the product of Example 21, Step E as the starting material the title compound was obtained.

### Step G 2,6-di-tert-butyl-4-{4-[1-(1H-imidazo[4,5-c]pyridin-1-ylacetyl)piperidin-4-yl]-1,3-thiazol-2-yl}phenol and 2,6-di-tert-butyl-4-{4-[1-(3H-imidazo[4,5-c]pyridin-3-ylacetyl)piperidin-4-yl]-1,3-thiazol-2-yl}phenol

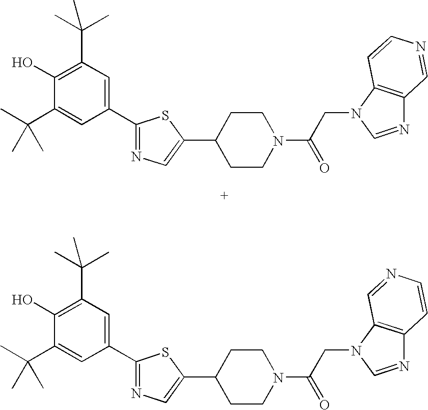

Using the method of Example 15, Step G with the product of Example 21, Step F as starting material the title compounds were obtained. ¹H NMR (CD₃OD): δ 8.93 (s, 1H), 8.36 (d, 1H, J=5.7 Hz), 8.31 (s, 1H), 7.72 (s, 1H), 7.64 (d, 1H, J=5.7 Hz), 7.11 (s, 1H), 5.47 (½AB, 1H, J=17.3 Hz), 5.41 (½AB, 1H, J=17.1 Hz), 4.57 (bd, 1H, J=10.4 Hz), 4.15 (bd, 1H, J=12.2 Hz), 3.42 (bt, 1H, J=13.0 Hz), 3.17 (tt, 1H, J=11.5, 3.2 Hz), 2.93 (dt, 1H, Jₜ=13.2, J_{d}=2.4 Hz), 2.24 (bd, 1H, J=12.7 Hz), 2.13 (bd, 1H, J=12.3 Hz), 1.92 (dquart, 1H, J_{q}=12.1, J_{d}=3.6 Hz), 1.74 (dquart, 1H, J_{q}=12.5, J_{d}=3.8 Hz), 1.47 (s, 18H). LRMS calc: 531.3 obs: 532.3 (M+H). ¹H NMR (CD₃OD): δ 8.88 (s, 1H), 8.37 (s overlapping d, 2H total, J_{d}=5.7 Hz), 7.73 (s overlapping d, 3H total, J_{d}=6.5 Hz), 7.11 (s, 1H), 5.54 (½AB, 1H, J=17.1 Hz), 5.48 (½AB, 1H, J=17.2 Hz), 4.57 (bd, 1H, J=13.8 Hz), 4.15 (bd, 1H, J=12.4 Hz), 3.43 (bt, 1H, J=13.2 Hz), 3.14 (tt, 1H, J=11.7, 3.6 Hz), 2.92 (bt, 1H, J=12.6 Hz), 2.24 (bd, 1H, J=12.4 Hz), 2.12 (bd, 1H, J=12.0 Hz), 1.92 (dquart, 1H, J_{q}=12.6, J_{d}=3.8 Hz), 1.74 (dquart, 1H, J_{q}=12.6, J_{d}=4.0 Hz), 1.47 (s, 18H). LRMS calc: 531.3 obs: 532.2 (M+H).

### Example 22

### Step A 2,6-di-tert-butyl-4-{4-[1-(3H-imidazo[-4,5-b]pyridin-3-ylacetyl)piperidin-4-yl]-1,3-thiazol-2-yl}phenol

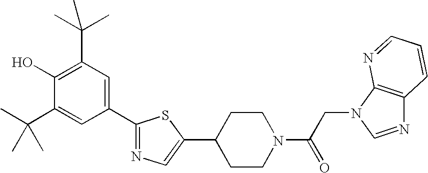

Using the method of Example 1, Step L with the products of Example 1, Step K and Example 21, Step E as the starting materials the title compound was obtained. ¹H NMR (CD₃OD): δ 8.37 (dd, 1H, J=4.8, 1.3 Hz), 8.36 (s, 1H), 8.10 (dd, 1H, J=8.0, 1.3 Hz), 7.72 (s, 2H), 7.35 (dd, 1H, J=7.9, 4.8 Hz), 7.10 (s, 1H), 5.42 (½AB, 1H, J=17.1 Hz), 5.36 (½AB, 1H, J=17.0 Hz), 4.56 (bd, 1H, J=11.4 Hz), 4.20 (bd, 1H, J=11.4 Hz), 3.42 (bt, 1H, J=11.9 Hz), 3.13 (btt, 1H, J=11.4, 3.2 Hz), 2.91 (bt, 1H, J=12.8 Hz), 2.25 (bd, 1H, J=12.7 Hz), 2.12 (bd, 1H, J=12.9 Hz), 1.93 (dquart, 1H, J_{q}=12.7, J_{d}=3.5 Hz), 1.73 (dquart, 1H, J_{q}=12.6, J_{d}=3.6 Hz), 1.47 (s, 18H). LRMS calc: 531.3 obs: 532.2 (M+H).

### Step B 2,6-di-tert-butyl-4-(4-{1-[(3,5-dimethyl-1H-1,2,4-triazol-1-yl)acetyl]piperidin-4-yl}-1,3-thiazol-2-yl)phenol

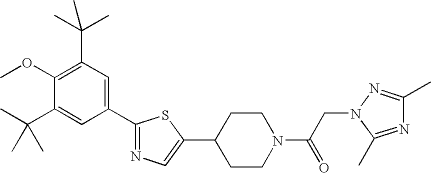

Using the method of Example 1, Step L with the products of Example 21, Step E and (3,5-dimethyl-1H-1,2,4-triazol-1-yl)acetic acid as the starting materials the title compound was obtained. ¹-H NMR (CD₃OD): δ 7.73 (s, 2H), 7.10 (s, 1H), 5.20 (½AB, 1H, J=17.2 Hz), 5.14 (½AB, 1H, J=17.0 Hz), 4.58 (bd, 1H, J=13.3 Hz), 4.08 (bd, 1H, J=14.1 Hz), 3.34 (bt, 1H, J=11.4 Hz), 3.13 (tt, 1H, J=11.9, 3.5 Hz), 2.91 (dt, 1H, Jₜ=12.8, J_{d}=2.3 Hz), 2.38 (s, 3H), 2.28 (s, 3H), 2.22 (bd, 1H, J=12.8 Hz), 2.14 (bd, 1H, J=12.8 Hz), 1.84 (dquart, 1H, J_{q}=12.8, J_{d}=4.1 Hz), 1.71 (dquart, 1H, J_{q}=12.6, J_{d}=4.1 Hz), 1.48 (s, 18H). LRMS calc: 509.3 obs: 510.3 (M+H).

### Example 23

### Step A [4-(hydroxymethyl)-5-methyl-1H-imidazol-1-yl]acetic acid methyl ester and [5-(hydroxymethyl)-4-methyl-1H-imidazol-1-yl]acetic acid methyl ester

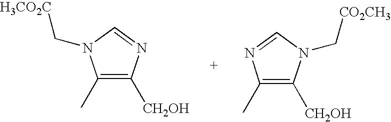

Using the method of Example 3, Step B and 4-(hydroxymethyl)-5-methyl-1H-imidazole as starting material the title compounds were obtained. The crude product was adsorbed onto silica gel and eluted without fractionation (10:1 CH₂Cl₂/MeOH). The eluant was evaporated and the derived residue chromatographed on Chiralcel OJ stationary phase (15% EtOH/heptane; λ=215 nM). The more mobile 5-hydroxymethyl and the less mobile 4-hydroxymethyl title isomers were isolated.

### Step B Potassium [4-(hydroxymethyl)-5-methyl-1H-imidazol-1-yl]acetate

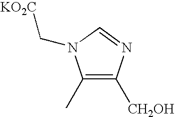

Using the method of Example 3, Step C and [4-(hydroxymethyl)-5-methyl-1H-imidazol-1-yl]acetic acid methyl ester (Example 23, Step A) as starting material the title compound was obtained.

### Step C [1-(2-{4-[2-(3,5-di-tert-butyl-4-methoxyphenyl)-1,3-thiazol-4-yl]piperidin-1-yl}-2-oxoethyl)-5-methyl4H-imidazol-4-yl]methanol

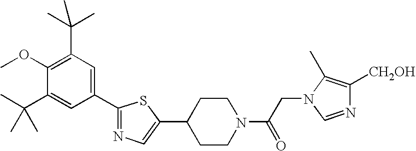

Using the method of Example 1, Step L and starting with the products of Example 1, Step H and Example 23, Step B the title compound was obtained. ¹H NMR (CD₃OD): δ 7.84 (s, 2H), 7.53 (s, 1H), 7.19 (s, 1H), 5.09 (½AB, 1H, J=17.1 Hz), 5.01 (½AB, 1H, J=17.3 Hz), 4.62 (bd, 1H, J=11.5 Hz), 4.52 (bs, 2H), 4.11 (bd, 1H, J=13.7 Hz), 3.75 (s, 3H), 3.38 (bt, 1H, J=14.4 Hz), 3.17 (bdt, 1H, Jₜ=11.7, J_{d}=3.7 Hz), 2.93 (bt, 1H, J=13.0 Hz), 2.26-2.15 (s overlapping bmult, 5H total), 1.87 (dquart, 1H, J_{q}=12.6, J_{d}=3.2 Hz), 1.75 (dquart, 1H, J_{q}=12.5, J_{d}=4.0 Hz), 1.50 (s, 181.1). LRMS calc: 538.3 obs: 539.3 (M+H).

### Step D Potassium [5-(hydroxymethyl)-4-methyl-1H-imidazol-1-yl]acetate

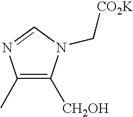

Using the method of Example 3, Step C and [5-(hydroxymethyl)-4-methyl-1H-imidazol-1-yl]acetic acid methyl ester (Example 23, Step A) as starting material the title compound was obtained.

### Step E [1-(2-{4-[2-(3,5-di-tert-butyl-4-methoxyphenyl)-1,3-thiazol-4-yl]piperidin-1-yl}-2-oxoethyl)-4-methyl-1H-imidazol-5-yl]methanol

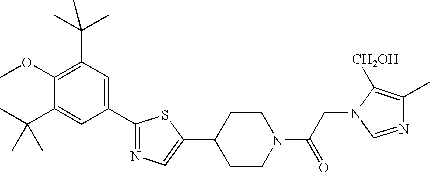

Using the method of Example 1, Step L and starting with the products of Example 1, Step H and Example 23, Step D the title compound was obtained. ¹H NMR (CD₃OD): δ 7.80 (s, 2H), 7.59 (s, 1H), 7.15 (s, 1H), 5.14 (½AB, 1H, J=17.0 Hz), 5.07 (½AB, 1H, J=17.1 Hz), 4.59 (bd, 1H, J=13.3 Hz), 4.49 (s, 2H), 4.07 (bd, 1H, J=13.9 Hz), 3.71 (s, 3H), 3.35 (dt, 1H, Jₜ=14.1, J_{d}=2.6 Hz), 3.13 (tt, 1H, J=11.7, 3.7 Hz), 2.89 (dt, 1H, Jₜ=13.0, J_{d}=2.3 Hz), 2.21-2.16 (s overlapping bd, 4H total), 212 (bd, 1H, J=13.8 Hz), 1.86 (dquart, 1H, J_{q}=12.6, J_{d}=3.7 Hz), 1.71 (dquart, 1H, J_{q}=12.6, J_{d}=3.8 Hz), 1.46 (s, 18H). LRMS calc: 538.3 obs: 539.3 (M+H).

### Example 24

### Step A 2-methylthio-1H-imidazole

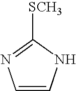

Using the method of Example 7, Step A and 2-mercaptoimidazole as starting material the title compound was obtained.

### Step B [2-(methylthio)-1H-imidazol-1-yl]acetic acid methyl ester

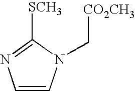

Using the method of Example 7, Step B and the product of Example 24, Step A as starting material the title compound was obtained as the sole product.

### Step C [2-(methylthio)-1H-imidazol-1-yl]acetic acid hydrochloride

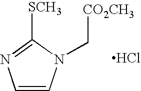

Using the method of Example 7, Step D and the product of Example 24, Step B as starting material the title compound was obtained.

### Step D 4-[2-(3,5-di-tert-butyl-4-methoxyphenyl)-1,3-thiazol-4-yl]-1-{2-(methylthio)-1H-imidazol-1-yl]acetyl}piperidine

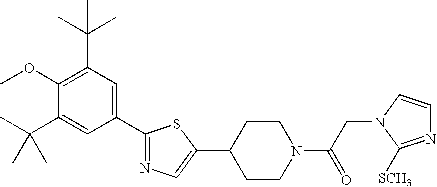

Using the method of Example 1, Step L with the products of Example 1, Step H and Example 24, Step C as starting materials the title compound was obtained. ¹H NMR (CD₃OD): δ 7.81 (s, 2H), 7.15 (s, 1H), 7.14 (d, 1H, J=1.2 Hz), 7.03 (d, 1H, J=1.3 Hz), 5.14 (½AB, 1H, J=16.9 Hz), 5.06 (½AB, 1H, J=16.7 Hz), 4.57 (bd, 1H, J=13.3 Hz), 4.09 (bd, 1H, J=13.5 Hz), 3.72 (s, 3H), 3.36 (bt, 1H, J=11.8 Hz), 3.14 (vbt, 1H, J=12.0 Hz), 2.90 (bt, 1H, J=12.9 Hz), 2.45 (s, 3H), 2.21 (bd, 1H, J=11.8 Hz), 2.13 (bd, 1H, J=12.2 Hz), 1.85 (dquart, 1H, J_{q}=12.6, J_{d}=3.7 Hz), 1.71 (dquart, 1H, J_{q}=13.0, J_{d}=3.5 Hz), 1.47 (s, 18H). LRMS calc: 540.3 obs: 541.4 (M+H).

### Step E [2-(methylsulfonyl)-1H-imidazol-1-yl]acetic acid methyl ester

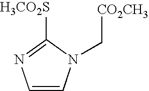

Using the method of Example 7, Step C and the product of Example 24, Step B as starting material the title compound was obtained.

### Step F [2-(methylsulfonyl)-1H-imidazol-1-yl]acetic acid hydrochloride

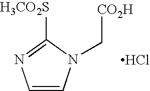

Using the method of Example 7, Step D and the product of Example 24, Step E as starting material the title compound was obtained.

### Step G 4-[2-(3,5-di-tert-butyl-4-methoxyphenyl)-1,3-thiazol-4-yl]-1-{2-(methylsulfonyl)-1H-imidazol-1-yl]acetyl}piperidine

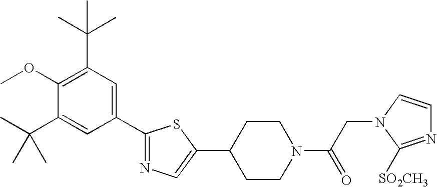

Using the method of Example 1, Step L with the products of Example 1, Step H and Example 24, Step F as the starting materials the title compound was obtained. ¹H NMR (CD₃OD): δ 7.81 (s, 2H), 7.32 (s, 1H), 7.20 (s, 1H), 7.14 (s, 1H), 5.47 (½AB, 1H, J=16.8 Hz), 5.36 (½AB, 1H, J=16.6 Hz), 4.57 (bd, 1H, J=13.9 Hz), 4.00 (bd, 1H, J=13.7 Hz), 3.71 (s, 3H), 3.34 (bt, 1H, J=13.1 Hz), 3.23 (s, 3H), 3.14 (bt, 1H, J=11.6 Hz), 2.90 (bt, 1H, J=11.9 Hz), 2.19 (bd, 1H, J=13.1 Hz), 2.12 (bd, 1H, J=12.9 Hz), 1.89 (dquart, 1H, J_{q}=12.4, J_{d}=3.3 Hz), 1.71 (dquart, 1H, J_{q}=12.5, J_{d}=3.5 Hz), 1.46 (s, 18H). LRMS calc: 572.3 obs: 573A (M+H).

### Example 25

### Step A tert-butyl methyl 2,2′(4-methyl-1H-imidazole-1,5-diyl)diacetate and tert-butyl methyl 2,2′-(5-methyl-1H-imidazole-1,4-diyl)diacetate

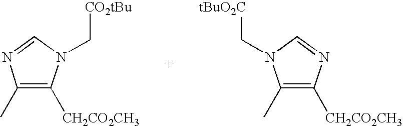

Using the method of Example 6, Step D and (4-methyl-1H-imidazol-5-yl)acetic acid methyl ester hydrochloride as the starting material the title compounds were obtained. Chiralcel OJ chromatography was performed to obtain the more mobile 1,5-diyl diester and the less mobile 1,4-diyl diester (7% EtOH/heptane; λ=225 nM).

### Step B [4-(2-methoxy-2-oxoethyl)-5-methyl-1H-imidazol-1-yl]acetic acid hydrotrifluoroaeetate

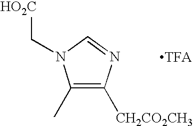

Using the method of Example 2, Step B and tert-butyl methyl 2,2′-(5-methyl-1H-imidazole-1,4-diyl)diacetate (Example 25, Step A) as starting material the title compound was obtained.

### Step C [1-(2-{4-[2-(3,5-di-tert-butyl-4-methoxyphenyl)-1,3-thiazol-4-yl]piperidin-1-yl}-2-oxoethyl)-5-methyl-1H-imidazol-4-yl]acetic acid methyl ester

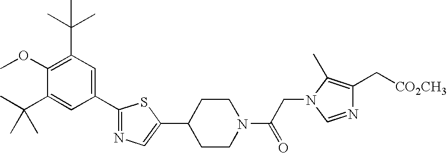

Using the method of Example 1, Step L with the product of Example 1, Step H and the product of Example 25, Step B as starting materials the title compound was obtained.

### Step D [1-(2-{4-[2-(3,5-di-tert-butyl-4-methoxyphenyl)-1,3-thiazol-4-yl]piperidin-1-yl}-2-oxoethyl)5-methyl-1H-imidazol-4-yl]acetic dihydrochloride

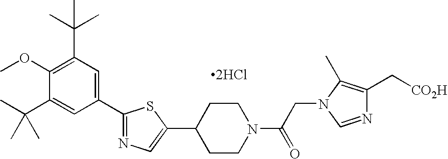

### Step G [1-(2-{4-[2-(3,5-di-tert-butyl-4-methoxyphenyl)-1,3-thiazol-4-yl]piperidin-1-yl}-2-oxoethyl)-4-methyl-1H-imidazol-5-yl]acetic dihydrochloride

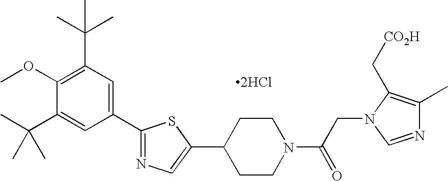

Using the method of Example 25, Step D with the product of Example 25, Step F as starting material the title compound was obtained. ¹H NMR (CD₃OD): δ 8.87 (s, 1H), 7.96 (s, 2H), 7.69 (s, 1H), 5.48 (½AB, 1H, J=16.9 Hz), 5.34 (½AB, 1H, J=17.0 Hz), 4.62 (bd, 1H, J=12.6 Hz), 4.10 (bd, 1H, J=12.8 Hz), 3.79 (s, 2H), 3.77 (s, 3H), 3.41 (vbquart, 2H, J=10.5 Hz), 2.95 (bt, 1H, J=12.0 Hz), 2.37 (bs, 3H), 2.28 (vbd, 1H, J=9.0 Hz), 2.20 (vbd, 1H, J=12.2 Hz), 2.02 (vbquart, 1H, J=10.5 Hz), 1.82 (vbquart, 1H, J=10.5 Hz), 1.51 (s, 18H). LRMS calc: 566.3 obs: 567.4 (M+H).

### Example 26

### Step A 4-[2-(3,5-di-tert-butyl-4-methoxyphenyl)-1,3-thiazol-4-yl]-1-[(3,5-dimethyl-1H-1,2A-triazol-1-yl)acetyl]piperidine

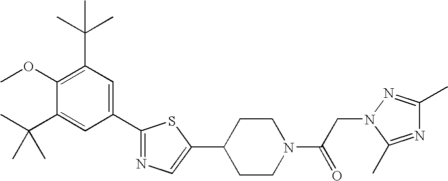

Using the method of Example I, Step L with the product of Example 1, Step H and (3,5-dimethyl-1H-1,2,4-triazol-1-yl)acetic acid as starting materials the title compound was obtained. ¹H NMR (CD₃OD): δ 7.83 (s, 2H), 7.17 (s, 1H), 5.22 (½AB, 1H, J=17.2 Hz), 5.14 (½AB, 1H, J=17.0 Hz), 4.59 (bd, 1H, J=12.8 Hz), 4.09 (bd, 1H, J=12.8 Hz), 3.74 (s, 3H), 3.36 (dt, 1H, Jₜ=13.4, J_{d}=2.2 Hz), 3.16 (tt, 1H, J=11.7, 3.0 Hz), 2.93 (bt, 1H, J=12.8 Hz), 2.39 (s, 3H), 2.30 (s, 3H), 2.23 (bd, 1H, J=13.0 Hz), 2.16 (bd, 1H, J=14.0 Hz), 1.86 (dquart, 1H, J_{q}=12.6, J_{d}=3.7 Hz), 1.73 (dquart, 1H, J_{q}=12.6, J_{d}=3.9 Hz), 1.49 (s, 18H). LRMS calc: 523.3 obs: 524.4 (M+H).

### Step B 4-[2-(3,5-di-tert-butylphenyl)-1,3-thiazol-4-yl]-1-[3,5-dimethyl-1H-1,2,4-triazol-1-yl)acetyl]piperidine

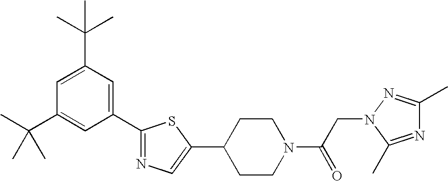

Using the method of Example 1, Step L with the product of Example 15, Step E and (3,5-dimethyl-1H-1,2,4-triazol-1-yl)acetic acid as starting materials the title compound was obtained. ¹H NMR (CD₃OD): δ 7.77 (d, 2H, J=1.6 Hz), 7.58 (t, 1H, J=1.6 Hz), 7.21 (s, 1H), 5.21 (½AB, 1H, J=17.2 Hz), 5.14 (½AB, 1H, J=17.0 Hz), 4.59 (bd, 1H, J=13.5 Hz), 4.09 (bd, 1H, J=13.9 Hz), 3.36 (dt, 1H, Jₜ=13.7, J_{d}=2.5 Hz), 3.17 (tt, 1H, J=11.4, 3.4 Hz), 2.92 (dt, 1H, Jₜ=13.0, J_{d}=2.7 Hz), 2.38 (s, 3H), 2.28 (s, 3H), 2.23 (bdd, 1H, J=14.2, 1.5 Hz), 2.16 (bd, 1H, J=12.3 Hz), 1.87 (dquart, 1H, J_{q}=13.0, J_{d}=3.9 Hz), 1.74 (dquart, 1H, J_{q}=12.6, J_{d}=4.1 Hz), 1.39 (s, 18H). LRMS calc: 493.3 obs: 494.3 (M+H).

### Example 27

### Step A (4-methyl-1H-imidazol-1-yl)acetic acid tert-butyl ester and (5-methyl-1H-imidazol-1-yl)acetic acid tert-butyl ester

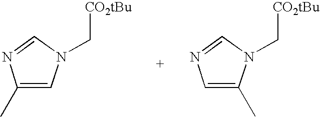

Using the method of Example 2, Step A with 4-methyl imidazole as starting material the title compounds were obtained. The mixture of isomers was chromatographed on Chiralcel OJ stationary phase (10% ethanol/heptane; λ=220 nM). The more mobile 4-methyl and less mobile 5-methyl title isomers were isolated.

### Step B (4-methyl-1H-imidazol-1-yl)acetic acid hydrotrifluoroacetate

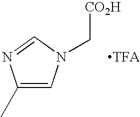

Using the method of Example 2, Step B with (4-methyl-1H-imidazol-1-yl)acetic acid tert-butyl ester (Example 27, Step A) as starting material the title compound was obtained.

### Step C 4-[2-(3,5-di-tert-butyl-4-methoxyphenyl)-1,3-thiazol-4-yl]-1-[(4-methyl-1-imidazol-1-yl)acetyl]piperidine

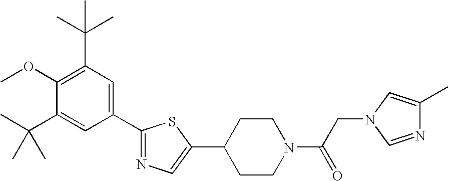

Using the method of Example 1, Step L with the products of Example 1, Step H and Example 27, Step B as starting materials the title compound was obtained. ¹H NMR (CD₃OD): δ 7.80 (s, 2H), 7.73 (bs, 1H), 7.14 (s, 1H), 6.87 (bs, 1H), 5.10 (½AB, 1H, J=16.8 Hz), 5.04 (½AB, 1H, J=17.0 Hz), 4.57 (bd, 1H, J=12.8 Hz), 4.01 (bd, 1H, J=13.0 Hz), 3.71 (s, 3H), 3.31 (bt, 1H, J=12.2 Hz), 3.12 (btt, 1H, J=11.6, 3.4 Hz), 2.88 (bt, 1H, J=12.8 Hz), 2.20 (s, 3H), 2.21 (s overlapping bd, 3H), 2.11 (bd, 1H, J=13.1 Hz), 1.80 (dquart, 1H, J_{q}=12.2, J_{d}=3.6 Hz), 1.71 (dquart, 1H, J_{q}=12.2, J_{d}=3.6 Hz), 1.46 (s, 18H). LRMS calc: 508.3 obs: 509.4 (M+H).

### Step D (5-methyl-1H-imidazol-1-yl)acetic acid hydrotrifluoroacetate

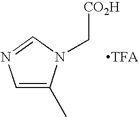

Using the method of Example 2, Step B with (5-methyl-1H-imidazol-1-yl)acetic acid tert-butyl ester (Example 27, Step A) as starting material the title compound was obtained.

### Step E 4-[2-(3,5-di-tert-butyl-4-methoxyphenyl)-1,3-thiazol-4-yl]-1-[(5-methyl-1-imidazol-1-yl)acetyl]piperidine

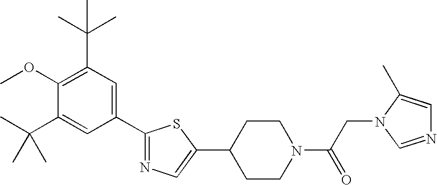

Using the method of Example 1, Step L with the products of Example 1, Step H and Example 27, Step D as starting materials the title compound was obtained. 1H NMR (CD₃OD): δ 7.80 (s, 2H), 7.52 (s, 1H), 7.15 (s, 1H), 6.69 (bs, 1H), 5.05 (½AB, 1H, J=17.3 Hz), 4.98 (½AB, 1H, J=17.1 Hz), 4.58 (bd, 1H, J=11.8 Hz), 4.09 (bd, 1H, J=13.4 Hz), 3.71 (s, 3H), 3.34 (dt, 1H, Jₜ=13.5, J_{d}=2.2 Hz), 3.13 (tt, 1H, J=11.8, 3.2 Hz), 2.89 (bt, 1H, J=12.9 Hz), 2.20 (bd, 1H, J=13.3 Hz), 2.13 (s, 3H), 2.11 (bd, 1H, J=13.5 Hz), 1.82 (dquart, 1H, J_{q}=12.5, J_{d}=3.6 Hz), 1.71 (dquart, 1H, J_{q}=12.6, J_{d}=3.8 Hz), 1.46 (s, 18H). LRMS calc: 508.3 obs: 509.4 (M+H).

### Example 28

### Step A (3-methyl-2-oxoimidazolidin-1-yl)acetic acid tert-butyl ester

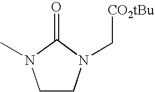

Using the method of Example 18, Step C with 1-methylimidazolidin-2-one as starting material the title compound was obtained.

### Step B (3-methyl-2-oxoimidazolidin-1-yl)acetic acid

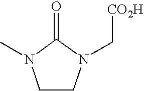

Using the method of Example 2, Step B with the product of Example 28, Step A as starting material the title compound was obtained.

### Step C 1-(2-{4-[2-(3,5-di-tert-butyl-4-methoxyphenyl)-1,3-thiazol-4-yl]piperidin-1-yl}-2-oxoethyl)-3-methylimidazolidin-2-one

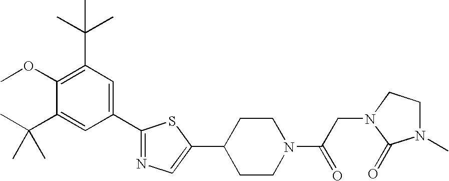

Using the method of Example 1, Step L with the products of Example 1, Step H and Example 28, Step B as starting materials the title compound was obtained. 1H NMR (CD₃OD): δ 7.81 (s, 2H), 7.14 (s, 1H), 4.59 (bd, 1H, J=13.0 Hz), 4.15 (½AB, 1 H, J=16.7 Hz), 4.06 (½AB, 1H, J=16.7 Hz), 4.01 (bd, 1H, J=13.9 Hz), 3.72 (s, 3H), 3.47 (mult, 2H), 3.41 (mult, 2H), 3.25 (dt, 1H, Jₜ=14.8, J_{d}=2.3 Hz), 3.12 (tt, 1H, J=11.7, 3.4 Hz), 2.83 (dt, 1H, Jₜ=12.8, J_{d}=2.3 Hz), 2.79 (s, 3H), 2.14 (bt, 2H, J=16.6 Hz), 1.78 (dquart, 1H, J_{q}=11.9, J_{d}=3.6 Hz), 1.68 (dquart, 1H, J_{q}=12.0, J_{d}=3.5 Hz), 1.47 (s, 18H). LRMS calc: 526.3 obs: 527.3 (M+H).

### Step D (2-oxo-1,3-oxazolidin-3-yl)acetic acid tert-butyl ester

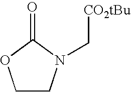

Using the method of Example 18, Step C with 1,3-oxazolidin-2-one as starting material the title compound was obtained.

### Step E (2-oxo-1,3-oxazolidin-3-yl)acetic acid

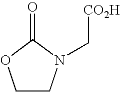

Using the method of Example 2, Step B with the product of Example 28, Step D as starting material the title compound was obtained.

### Step F 3-(2-{4-[2-(3,5-di-tert-butyl-4-methoxyphenyl)-1,3-thiazol-4-yl]piperidin-1-yl}-2-oxoethyl)-1,3-oxazolidin-2-one

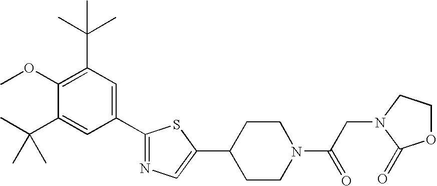

Using the method of Example 1, Step L with the products of Example 1, Step H and Example 28, Step E as starting materials the title compound was obtained. ¹H NMR (CD₃OD): δ 7.83 (s, 2H), 7.17 (s, 1H), 4.62 (bd, 1H, J=13.3 Hz), 4.43 (t, 2H, J=8.2 Hz), 4.27 (½AB, 1H, J=17.2 Hz), 4.20 (½AB, 1H, J=17.0 Hz), 4.00 (bd, 1H, J=13.5 Hz), 3.74 (s, 3H), 3.73 (t, 2H, J=8.1 Hz), 3.29 (bt, 1H, J=12.5 Hz), 3.13 (tt, 1H, J=11.7, 3.4 Hz), 2.89 (dt, 1H, Jₜ=12.8, J_{d}=2.3 Hz), 2.17 (bt, 2H, J=17.2 Hz), 1.81 (dquart, 1H, J_{q}=12.6, J_{d}=3.9 Hz), 1.72 (dquart, 1H, J_{q}=12.5, J_{d}=3.7 Hz), 1.49 (s, 18H). LRMS calc: 513.3 obs: 514.4 (M+H).

### Example 29

### Step A 4-[2-(3,5-di-tert-butyl-4-methoxyphenyl)-1,3-thiazol-4-yl]-1-[(3,5-dimethyl-1H-pyrazol-1-yl)acetyl]piperidine

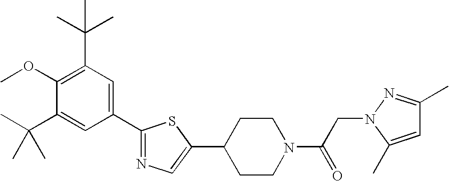

Using the method of Example 1, Step L with the product of Example 1, Step H and (3,5-dimethyl-1H-pyrazol-1-yl)acetic acid as starting materials the title compound was obtained. ¹H NMR (CDCl₃): δ 7.84 (s, 2H), 7.31 (s, 1H), 6.86 (s, 1H), 4.99 (½AB, 1H, J=16.2 Hz), 4.92 (½AB, 1H, J=16.4 Hz), 4.70 (bd, 1H, J=13.4 Hz), 4.09 (bd, 1H, J=13.4 Hz), 3.75 (s, 3H), 3.28 (bt, 1H, J=12.4 Hz), 3.10 (tt, 1H, J=11.7, 3.7 Hz), 2.86 (dt, 1H, Jₜ=12.8, J_{d}=2.1 Hz), 2.29 (s, 3H), 2.27 (s, 3H), 2.22 (bd, 1H, J=13.0 Hz), 2.18 (bd, 1H, J=13.0 Hz), 1.80-1.64 (mult, 2H), 1.52 (s, 18H). LRMS calc: 522.3 obs: 523.3 (M+H).

### Step B 4-[2-(3,5-di-tert-butyphenyl)-1,3-thiazol-4-yl]-1-[(3,5-dimethyl-1H-pyrazol-1-yl)acetyl]piperidine

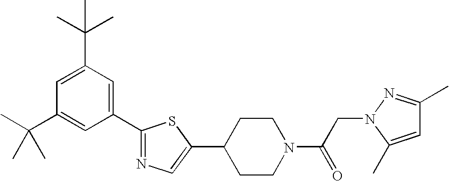

Using the method of Example 1, Step L with the product of Example 15, Step E and (3,5-dimethyl-1H-pyrazol-1-yl)acetic acid as starting materials the title compound was obtained. ¹H NMR (CD₃OD): δ 7.77 (d, 2H, J=1.6 Hz), 7.58 (t, 1H, J=1.6 Hz), 7.21 (s, 1H), 6.40 (s, 1H), 5.21 (½AB, 1H, J=17.2 Hz), 5.14 (½AB, 1H, J=17.0 Hz), 4.59 (bd, 1H, J=13.5 Hz), 4.09 (bd, 1H, J=13.9 Hz), 3.36 (dt, 1H, Jₜ=13.7, J_{d}=2.5 Hz), 3.17 (tt, 1H, J=11.4, 3.4 Hz), 2.92 (dt, 1H, Jₜ=13.0, J_{d}=2.7 Hz), 2.38 (s, 3H), 2.28 (s, 3H), 2.23 (bdd, 1H, J=14.2, 1.5 Hz), 2.16 (bd, 1H, J=12.3 Hz), 1.87 (dquart, 1H, J_{q}=13.0, J_{d}=3.9 Hz), 1.74 (dquart, 1H, J_{q}=12.6, J_{d}=4.1 Hz), 1.39 (s, 18H). LRMS calc: 493.3 obs: 494.4 (M+H).

### Example 30

### Step A 1H-pyrrolo[2,3-b]pyridin-1-ylacetic acid tert-butyl ester

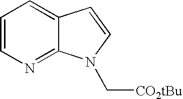

Using the method of Example 4, Step B and 1H-pyrrolo[2,3-b]pyridine as the starting material the title compound was obtained.

### Step B 1H-pyrrolo[2,3-b]pyridin-1-ylacetic acid hydrotrifluoroacetate

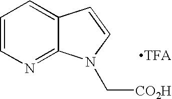

Using the method of Example 2, Step B and the product of Example 30, Step A as starting material the title compound was obtained.

### Step C 1-(2-{4-[2-(3,5-di-tert-butyl-4-methoxyphenyl)-1,3-thiazol-4-yl]piperidin-1-yl}-2-oxoethyl)-1H-pyrrolo[2,3-b]pyridine

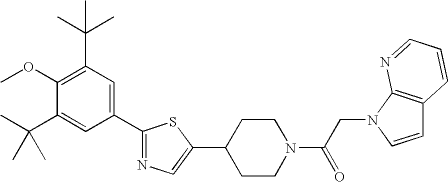

Using the method of Example 1, Step L with the products of Example 1, Step H and Example 30, Step B as starting materials the title compound was obtained. ¹H NMR (CD₃OD): δ 8.22 (d, 1H, J=3.7 Hz), 8.02 (dd, 1H, J=6.8, 3.4 Hz), 7.85 (s, 2H), 7.39 (quart, 1H, J=3.3 Hz), 7.19 (bs, 1H), 7.15 (dd, 1H, J=7.7, 4.8 Hz), 6.58 (t, 1H, J=3.3 Hz), 5.37 (½AB, 1H, J=16.9 Hz), 5.28 (½AB, 1H, J=16.9 Hz), 4.61 (bd, 1H, J=13.3 Hz), 4.25 (bd, 1H, J=13.4 Hz), 3.76 (s, 3H), 3.42 (bt, 1H, J=12.0 Hz), 3.17 (vbt, 1H, J=12.1 Hz), 2.92 (bt, 1H, J=13.0 Hz), 2.23 (bd, 1H, J=12.8 Hz), 2.14 (bd, 1H, J=12.9 Hz), 1.91 (dquart, 1H, J_{q}=12.3, J_{d}=3.0 Hz), 1.76 (dquart, 1H, J_{q}=12.2, J_{d}=3.1 Hz), 1.51 (s, 18H). LRMS calc: 544.3 obs: 545.3 (M+H).

### Step D 1-{2-[4-(2-{3-tert-butyl-5-[(trifluoromethyl)thio]phenyl}-1,3-thiazol-4-yl)piperidin-1-yl]-2-oxoethyl}-1H-pyrrolo[2,3-b]pyridine

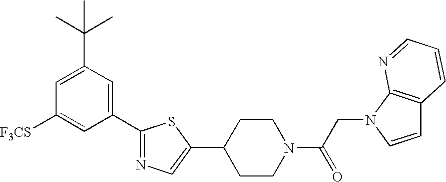

Using the method of Example 1, Step L with the products of Example 11, Step L and Example 30, Step B as starting materials the title compound was obtained. ¹H NMR (CD₃OD): δ 8.23 (dd, 1H, J=4.8, 1.4 Hz), 8.17 (t, 1H, J=1.6 Hz), 8.08 (s, 1H), 8.03 (dd, 1H, J=7.8, 1.4 Hz), 7.82 (s, 1H), 7.40 (d, 1H, J=3.6 Hz), 7.32 (s, 1H), 7.15 (dd, 1H, J=7.7, 4.8 Hz), 6.57 (d, 1H, J=3.5 Hz), 5.38 (½AB, 1H, J=16.9 Hz), 5.29 (½AB, 1H, J=16.9 Hz), 4.61 (bd, 1H, J=13.2 Hz), 4.26 (bd, 1H, J=13.7 Hz), 3.43 (dt, 1H, Jₜ=12.5, J_{d}=2.6 Hz), 3.21 (vbt, 1H, J=11.7 Hz), 2.93 (bt, 1H, J=11.8 Hz), 2.24 (bd, 1H, J=12.8 Hz), 2.16 (bd, 1H, J=12.8 Hz), 1.95 (dquart, 1H, J_{q}=12.8, J_{d}=3.7 Hz), 1.79 (dquart, 1H, J_{q}=12.5, J_{d}=3.6 Hz), 1.44 (s, 9H). LRMS calc: 558.2 obs: 559.2 (M+H).

### Example 31

### Step A 1-(2-{4-[2-(3,5-di-tert-butyl-4-methoxyphenyl)-1,3-thiazol-4-yl]piperidin-1-yl}-2-oxoethyl)-1H-benzimidazole

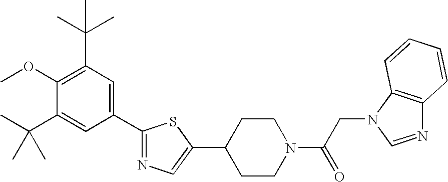

Using the method of Example 1, Step L with the product of Example 1, Step H and 1H-benzimidazol-1-ylacetic acid as the starting materials the title compound was obtained. ¹H NMR (CD₃OD): δ 8.12 (s, 1H), 7.81 (s, 2H), 7.67 (d, 1H, J=7.5 Hz), 7.48 (d, 1H, J=8.4 Hz), 7.32-7.25 (mult, 2H), 7.16 (s, 1H), 5.39 (½AB, 1H, J=17.3 Hz), 5.33 (½AB, 1H, J=17.3 Hz), 4.58 (bd, 1H, J=12.7 Hz), 4.19 (bd, 1H, J=12.6 Hz), 3.72 (s, 3H), 3.41 (bt, 1H, J=13.1 Hz), 3.15 (vbt, 1H, J=11.8 Hz), 2.91 (bt, 1H, J=11.6 Hz), 2.24 (bd, 1H, J=12.9 Hz), 2.12 (bd, 1H, J=13.6 Hz), 1.88 (dquart, 1H, J_{q}=13.1, J_{d}=2.8 Hz), 1.73 (dquart, 1H, J_{q}=12.9, J_{d}=4.1 Hz), 1.47 (s, 18H). LRMS calc: 544.3 obs: 545.4 (M+H).

### Step B 1-(2-{4-[2-(3,5-di-tert-butyl-4-methoxyphenyl)-1,3-thiazol-4-yl]piperidin-1-yl}-2-oxoethyl)-2-methyl-1H-benzimidazole

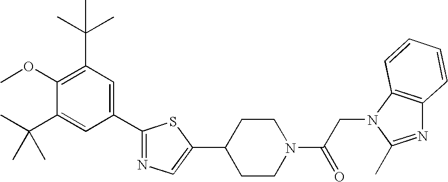

Using the method of Example 1, Step L with the product of Example 1, Step H and (2-methyl-1H-benzimidazol-1-yl)acetic acid as the starting materials the title compound was obtained. ¹H NMR (CD₃OD): δ 7.82 (s, 2H), 7.54 (d, 1H, J=8.5 Hz), 7.38 (d, 1H, J=8.5 Hz), 7.23-7.19 (mult, 2H), 7.18 (s, 1H), 5.31 (½AB, 1H, J=17.6 Hz), 5.33 (½AB, 1H, J=17.7 Hz), 4.56 (bd, 1H, J=11.0 Hz), 4.22 (bd, 1H, J=13.0 Hz), 3.72 (s, 3H), 3.44 (dt, 1H, Jₜ=11.9, J_{d}=2.4 Hz), 3.16 (bt, 1H, J=11.9 Hz), 2.92 (dt, 1H, Jₜ=12.6, J_{d}=2.6 Hz), 2.52 (s, 3H), 2.26 (bd, 1H, J=11.0 Hz), 2.13 (bd, 1H, J=11.5 Hz), 1.92 (dquart, 1H, J_{q}=11.5, J_{d}=3.6 Hz), 1.74 (dquart, 1H, J_{q}=12.4, J_{d}=4.1 Hz), 1.47 (s, 18H). LRMS calc: 558.3 obs: 559.4 (M+H).

### Step C 1-{2-[4-(2-{3-tert-butyl-5-[(trifluoromethyl)thio]phenyl}-1,3-thiazol-4-yl)piperidin-1-yl]-2-oxoethyl}-1H-benzimidazole

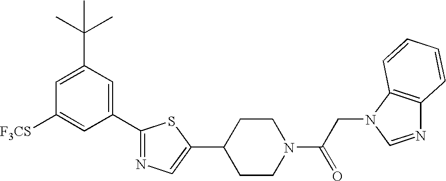

Using the method of Example 1, Step L with the product of Example 11, Step L and 1H-benzimidazol-1-ylacetic acid as starting materials the title compound was obtained. ¹H NMR (CD₃OD): δ 8.60 (bs, 1H), 8.13 (s, 1H), 8.05 (s, 1H), 7.79 (s, 1H), 7.74 (d, 1H, J=7.5 Hz), 7.64 (d, 1H, J=8.0 Hz), 7.45-7.40 (mult, 2H), 7.30 (s, 1H), 5.52 (½AB, 1H, J=17.3 Hz), 5.45 (½AB, 1H, J=17.4 Hz), 4.58 (bd, 1H, J=13.0 Hz), 4.18 (bd, 1H, J=14.3 Hz), 3.44 (dt, 1H, Jₜ=14.7, J_{d}=2.3 Hz), 3.20 (tt, 1H, J=11.7, 3.6 Hz), 2.94 (dt, 1H, Jₜ=13.0, J_{d}=2.6 Hz), 2.26 (bd, 1H, J=13.6 Hz), 2.16 (dd, 1H, J=12.8, 5.5 Hz), 1.95 (dquart, 1H, J_{q}=12.6, J_{d}=3.7 Hz), 1.78 (dquart, 1H, J_{q}=12.4, J_{d}=3.7 Hz), 1.40 (s, 9H). LRMS calc: 558.2 obs: 559.2 (M+H).

### Step D 1-{2-[4-(2-{3-tert-butyl-5-[(trifluoromethyl)thio]phenyl}-1,3-thiazol-4-yl)piperidin-1-yl]-2-oxoethyl}-2-methyl-1H-benzimidazole

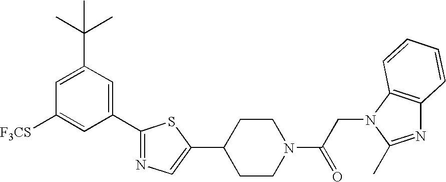

Using the method of Example 1, Step L with the product of Example 11, Step L and (2-methyl-1H-benzimidazol-1-yl)acetic acid as the starting materials the title compound was obtained. ¹H NMR (CD₃OD): δ 8.16 (s, 1H), 8.07 (s, 1H), 7.81 (s, 1H), 7.57 (dd, 1H, J=8.5, 1.6 Hz), 7.40 (dd, 1H, J=8.4, 1.4 Hz), 7.33 (s, 1H), 7.26-7.21 (mult, 2H), 5.32 (½AB, 1H, J=17.8 Hz), 5.25 (½AB, 1H, J=17.7 Hz), 4.58 (bd, 1H, J=12.1 Hz), 4.24 (bd, 1H, J=13.2 Hz), 3.46 (bt, 1H, J=11.9 Hz), 3.21 (tt, 1H, J=11.7, 3.5 Hz), 2.91 (dt, 1H, Jₜ=12.8, J_{d}=1.8 Hz), 2.54 (s, 3H), 2.29 (bd, 1H, J=12.1 Hz), 2.16 (bd, 1H, J=13.2 Hz), 1.97 (dquart, 1H, J_{q}=12.8, J_{d}=3.8 Hz), 1.78 (dquart, 1H, J_{q}=12.8, J_{d}=3.9 Hz), 1.42 (s, 9H). LRMS calc: 572.2 obs: 573.3 (M+H).

### Example 32

### Step A [5-methyl-3-(trifluoromethyl)-1H-pyrazol-1-yl]acetic acid tert-butyl ester

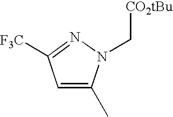

Using the method of Example 4, Step B with 5-methyl-3-(trifluoromethyl)-1H-pyrazole as the starting material the title compound was obtained as the sole product.

### Step B [5-methyl-3-(trifluoromethyl)-1H-pyrazol-1-yl]acetic acid hydrotrifluoroacetate

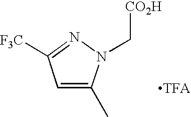

Using the method of Example 2, Step B with the product of Example 32, Step A as the starting material the title compound was obtained.

### Step C 4-[2-(3,5-di-tert-butylphenyl-1,3-thiazol-4-yl]-1-{[5-methyl-3-(trifluoromethyl)-1H-pyrazol-1-yl]acetyl}piperidine

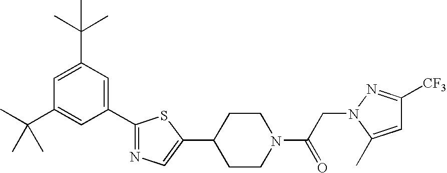

Using the method of Example 1, Step L with the products of Example 15, Step E and Example 32, Step B as the starting materials the title compound was obtained. ¹H NMR (CD₃OD): δ 7.79 (d, 2H, J=1.8 Hz), 7.60 (t, 1H, J=1.7 Hz), 7.23 (s, 1H), 6.44 (s, 1H), 5.29 (½AB, 1H, J=17.0 Hz), 5.21 (½AB, 1H, J=17.0 Hz), 4.59 (bs, 1H), 4.14 (bd, 1H, J=13.7 Hz), 3.40 (bt, 1H, J=11.8 Hz), 3.19 (tt, 1H, J=11.9, 3.5 Hz), 2.95 (bt, 1H, J=11.7 Hz), 2.33 (s, 3H), 2.25 (bd, 1H, J=13.5 Hz), 2.18 (bd, 1H, J=13.0 Hz), 1.88 (dquart, 1H, J_{q}=12.6, J_{d}=4.1 Hz), 1.76 (dquart, 1H, J_{d}=12.6, J_{d}=4.1 Hz), 1.40 (s, 18H). LRMS calc: 546.3 obs: 547.4 (M+H).

### Step D 1-{[3,5-bis(trifluoromethyl)-1H-pyrazol-1-yl]acetyl}-4-[2-(3,5-di-tert-butylphenyl)-1,3-thiazol-4-yl]piperidine

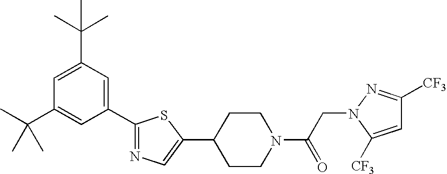

Using the method of Example 1, Step L with the product of Example 15, Step E and [3,5-bis(trifluoromethyl)-1H-pyrazol-1-yl]acetic acid as starting materials the title compound was obtained. ¹H NMR (CD₃OD): δ 7.78 (bs, 2H), 7.58 (bs, 1H), 7.23 (s, 1H), 7.22 (s, 1H), 5.51 (½AB, 1H, J=16.7 Hz), 5.44 (½AB, 1H, J=16.9 Hz), 4.59 (bs, 1H), 4.07 (bd, 1H, J=13.7 Hz), 3.39 (bt, 1H, J=12.6 Hz), 3.18 (bt, 1H, J=11.9 Hz), 2.94 (bt, 1H, J=12.1 Hz), 2.24 (bd, 1H, J=13.3 Hz), 2.16 (bd, 1H, J=13.0 Hz), 1.86 (dquart, 1H, J_{q}=12.3, J_{d}=4.0 Hz), 1.75 (dquart, 1H, J_{q}=12.2, J_{d}=3.8 Hz), 1.39 (s, 18H). LRMS calc: 600.2 obs: 601.3 (M+H).

### Step E 1-[(4-bromo-3,5-dimethyl-1H-pyrazol-1-yl)acetyl]-4-[2-(3,5-di-tert-butylphenyl)-1,3-thiazol-4-yl]piperidine

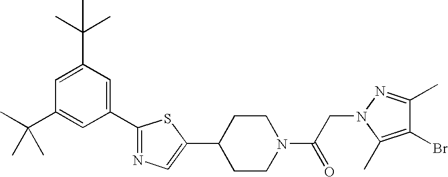

Using the method of Example 1, Step L with the product of Example 15, Step E and (4-bromo-3,5-dimethyl-1H-pyrazol-1-yl)acetic acid as starting materials the title compound was obtained. 1H NMR (CD₃OD): δ 7.79 (bs, 2H), 7.59 (bs, 1H), 7.22 (s, 1H), 5.16 (½AB, 1H, J=17.1 Hz), 5.09 (½AB, 1H, J=17.0 Hz), 4.60 (bd, 1H, J=11.5 Hz), 4.12 (bd, 1H, J=12.8 Hz), 3.37 (bt, 1H, J=12.2 Hz), 3.18 (bt, 1H, J=11.5 Hz), 2.92 (bt, 1H, J=12.6 Hz), 2.24-2.15 (2s overlapping 2bd, 8H total), 1.87 (dquart, 1H, J_{q}=11.4, J_{d}=2.9 Hz), 1.75 (dquart, 1H, J_{q}=11.8, J_{d}=3.2 Hz), 1.40 (s, 18H). LRMS calc: 570.2 obs: 571.3 (M+H).

### Step F 1-[(4-iodo-3,5-dimethyl-1H-pyrazol-1-yl)acetyl]-4-[2-(3,5-di-tert-butylphenyl)-1,3-thiazol-4-yl]piperidine

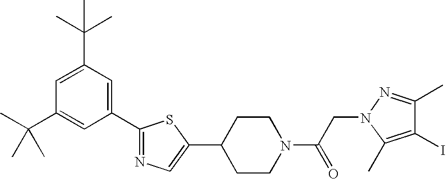

Using the method of Example 1, Step L with the product of Example 15, Step E and (4-iodo-3,5-dimethyl-1H-pyrazol-1-yl)acetic acid as starting materials the title compound was obtained. ¹H NMR (CDCl₃): δ 7.78 (d, 2H, J=1.6 Hz), 7.52 (t, 1H, J=1.7 Hz), 6.88 (s, 1H), 5.01 (½AB, 1H, J=16.3 Hz), 4.96 (½AB, 1H, J=16.4 Hz), 4.68 (bd, 1H, J=13.3 Hz), 4.03 (bd, 1H, J=13.5 Hz), 3.29 (dt, 1H, Jₜ=14.6, J_{d}=2.5 Hz), 3.12 (tt, 1H, J=11.7, 3.4 Hz), 2.85 (dt, 1H, Jₜ=13.0, J_{d}=2.5 Hz), 2.30 (s, 3H), 2.25 (s overlapping bd, 4H total), 2.17 (bd, 1H, J=12.4 Hz), 1.81-1.70 (mult, 2H), 1.40 (s, 18H). LRMS calc: 618.2 obs: 619.3 (M+H).

### Example 33

### Step A (2-oxoimidazolidin-1-yl)acetic acid tert-butyl ester

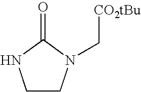

Using the method of Example 18, Step C and imidazolidin-2-one as the starting material the tile compound was obtained. Purification was carried out on Chiralcel AS stationary phase (10% EtOH/heptane; λ=210 nM).

### Step B (2-oxoimidazolidin-1-yl)acetic acid

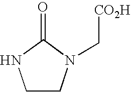

Using the method of Example 2, Step B and the product of Example 33, Step A as the starting material the title compound was obtained.

### Step C 1-(2-{4-[2-(3,5-di-tert-butyl-4-methoxyphenyl)-1,3-thiazol-4-yl]piperidin-1-yl}-2-oxoethyl)imidazolidin-2-one

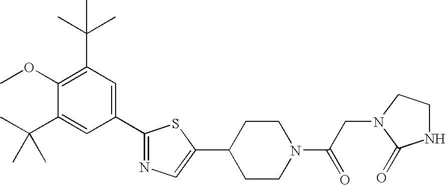

Using the method of Example 1, Step L with the products of Example 1, Step H and Example 33, Step B as the starting materials the title compound was obtained. ¹H NMR (CD₃OD): δ 7.80 (s, 2H), 7.13 (s, 1H), 4.58 (bd, 1H, J=13.0 Hz), 4.14 (½AB, 1H, J=16.7 Hz), 4.05 (½AB, 1H, J=16.7 Hz), 4.00 (bd, 1H, J=13.1 Hz), 3.71 (s, 3H), 3.56 (dpent, 2H, Jₚ=8.2, J_{d}=4.6 Hz), 3.44 (t, 2H, J=8.0 Hz), 3.24 (dt, 1H, 1H, J₅=14.3, J_{d}=2.4 Hz), 3.09 (tt, 1H, J=11.7, 3.6 Hz), 2.83 (dt, 1H, Jₜ=13.0, J_{d}=2.7 Hz), 2.12 (bmult, 2H), 1.77 (dquart, 1H, J_{q}=12.4, J_{d}=3.7 Hz), 1.67 (dquart, 1H, J_{q}=12.3, J_{d}=3.5 Hz), 1.46 (s, 18H). LRMS calc: 512.3 obs: 513.3 (M+H).

### Example 34

### Step A [2-(hydroxymethyl)-1H-imidazol-1-yl]acetic acid methyl ester

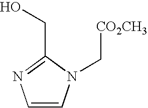

A solution of 1H-imidazol-2-ylmethanol hydrochloride (986 mg; 7.33 mmol) in dry DMSO (20 mL) was treated with methyl bromoacetate (696 μL; 7.33 mmol) and cesium carbonate (5.97 g; 18.32 mmol). The mixture was stirred at ambient temperature for 16 h. The reaction mixture was diluted with isopropyl acetate (100 mL) and filtered. The filtrate was evaporated onto silica gel. The silica gel was eluted (40:1 CH₂Cl₂/MeOH) without fractionation to desorb DMSO. The silica gel was then eluted without fractionation (100:10:1 CH₂Cl₂/MeOH/Et₃N) to recover the title compound. The second eluant was evaporated and chromatographed over Chiralcel AD stationary phase (15% EtOH/heptane; λ=220 nM). The second eluting peak was recovered, affording the title compound (109 mg).

### Step B Potassium [2-(hydroxymethyl)-1H-imidazol-1-yl]acetate

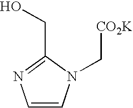

Using the method of Example 3, Step C and the product of Example 34, Step A as the starting material the title compound was obtained.

### Step C [1-(2-{4-[2-(3,5-di-tert-butyl-4-methoxyphenyl)-1,3-thiazol-4-yl]piperidin-1-yl}-2-oxoethyl)-1H-imidazol-2-yl]methanol

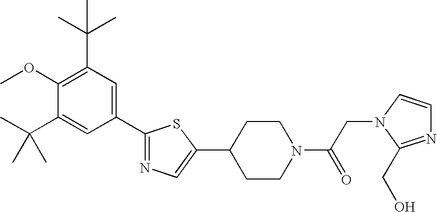

Using the method of Example 1, Step L with the products of Example 1, Step H and Example 34, Step B as the starting materials the title compound was obtained. ¹H NMR (CD₃OD): δ 7.84 (s, 2H), 7.17 (s, 1H), 7.08 (bs, 1H), 6.93 (bs, 1H), 5.23 (½AB, 1H, J=17.2 Hz), 5.16 (½AB, 1H, J=17.3 Hz), 4.62 (s overlapping bd, 3H total, J_{d}=14.5 Hz), 4.09 (bd, 1H, J=13.9 Hz), 3.74 (s, 3H), 3.36 (dt, 1H, Jₜ=14.2, J_{d}=2.1 Hz), 3.15 (tt, 1H, J=11.9, 3.4 Hz), 2.91 (dt, 1H, Jₜ=12.8, J_{d}=2.3 Hz), 2.22 (bd, 1H, J=12.8 Hz), 2.15 (bd, 1H, J=14.2 Hz), 1.87 (dquart, 1H, J_{q}=12.6, J_{d}=3.6 Hz), 1.74 (dquart, 1H, J_{q}=12.6, J_{d}=3.9 Hz), 1.49 (s, 18H). LRMS calc: 524.3 obs: 525.3 (M+H).

### Step D [2-(hydroxymethyl)-1H-benzimidazol-1-yl]acetic acid methyl ester

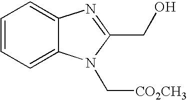

Using the method of Example 3, Step B and 1H-benzimidazol-2-ylmethanol as the starting material the title compound was obtained as the sole product. Purification was performed by silica gel chromatography (preparative TLC; 20:1 CH₂Cl₂/MeOH).

### Step E Potassium [2-(hydroxymethyl)-1H-benzimidazol-1-yl]acetate

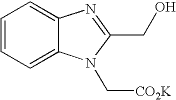

Using the method of Example 3, Step C and the product of Example 34, Step D as the starting material the title compound was obtained.

### Step F [1-(2-{4-[2-(3,5-di-tert-butyl-4-methoxyphenyl)-1,3-thiazol-4-yl]piperidin-1-yl}-2-oxoethyl)-1H-benzimidazol-2-yl]methanol

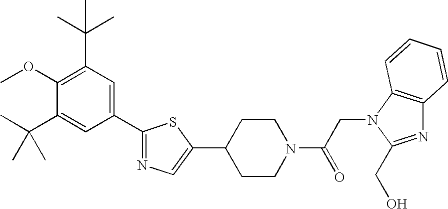

Using the method of Example 1, Step L with the products of Example 1, Step H and Example 34, Step E as the starting materials the title compound was obtained. ¹H NMR (CD₃OD): δ 7.84 (s, 2H), 7.64 (d, 1H, J=7.8 Hz), 7.46 (d, 1H, J=7.8 Hz), 7.31-7.24 (mult, 2H), 7.18 (s, 1H), 5.47 (½AB, 1H, J=18.0 Hz), 5.39 (½AB, 1H, J=17.8 Hz), 4.86 (s, 2H), 4.57 (bd, 1H, J=13.5 Hz), 4.22 (bd, 1H, J=13.9 Hz), 3.74 (s, 3H), 3.43 (bt, 1H, J=11.9 Hz), 3.17 (tt, 1H, J=11.7, 3.4 Hz), 2.92 (bt, 1H, J=11.9 Hz), 2.26 (bd, 1H, J=12.6 Hz), 2.14 (bd, 1H, J=13.0 Hz), 1.93 (dquart, 1H, J_{q}=12.3, J_{d}=3.4 Hz), 1.73 (dquart, 1H, J_{q}=12.8, J_{d}=3.9 Hz), 1.49 (s, 18H). LRMS calc: 574.3 obs: 575.5 (M+H).

### Example 35

### Step A 4-[2-(3,5-di-tert-butyl-4-methoxyphenyl)-1,3-thiazol-4-yl]-1-{[2-(trifluoromethyl)-1H-imidazol-1-yl]acetyl}piperidine

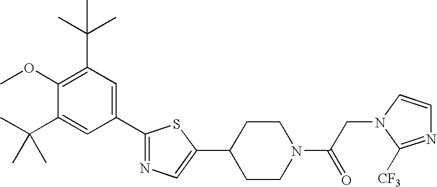

Using the method of Example 1, Step L with the product of Example 1, Step H and [2-(trifluoromethyl)-1H-imidazol-1-yl]acetic acid as the starting materials the title compound was obtained. ¹H NMR (CDCl₃): δ 7.73 (s, 2H), 7.19 (dd, 1H, J=3.4, 0.9 Hz), 7.13 (dd, 1H, J=3.6, 1.0 Hz), 6.96 (s, 1H), 5.01 (½AB, 1H, 16.7 Hz), 4.90 (½AB, 1H, J=16.9 Hz), 4.73 (bd, 1H, J=13.9 Hz), 3.88 (bd, 1H, J=14.4 Hz), 3.75 (s, 3H), 3.42 (bt, 1H, J=12.2 Hz), 3.20 (bt, 1H, J=11.6 Hz), 2.91 (bt, 1H, J=11.8 Hz), 2.48 (bd, 1H, J=13.1 Hz), 2.23 (bd, 1H, J=13.5 Hz), 1.89-1.70 (vbmult, 2H), 1.52 (s, 18H). LRMS calc: 562.3 obs: 563.4 (M+H).

### Step B 4-[2-(3,5-di-tert-butyl-4-methoxyphenyl)-1,3-thiazol-4-yl]-1-{[2-methyl-1H-imidazol-1-yl]acetyl}piperidine

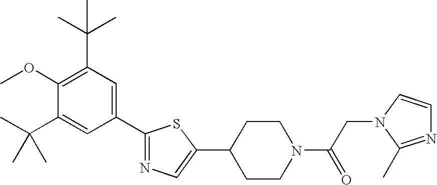

Using the method of Example 1, Step L with the product of Example 1, Step H and [2-methyl-1H-imidazol-1-yl]acetic acid as the starting materials the title compound was obtained. ¹H NMR (CDCl₃): δ 7.83 (s, 2H), 6.99 (bs, 1H), 6.87 (bs, 2H), 4.75-4.70 (bs overlapping bd, 3H), 3.91 (bd, 1H, J=14.8 Hz), 3.75 (s, 3H), 3.33 (bt, 1H, J=12.5 Hz), 3.13 (vbt, 1H, J=11.6 Hz), 2.90 (bt, 1H, J=11.8 Hz), 2.40 (bs, 3H), 2.29 (bd, 1H, J=13.0 Hz), 2.21 (bd, 1H, J=13.1 Hz), 1.77 (bdhex, 2H, Jₕ=12.6, J_{d}=4.0 Hz), 1.51 (s, 18H). LRMS calc: 508.3 obs: 509.4 (M+H).

### Step 4-[2-(3,5-di-tert-butyl-4-methoxyphenyl)-1,3-thiazol-4-yl]-1-(1H-imidazol-1-ylacetyl)piperidine

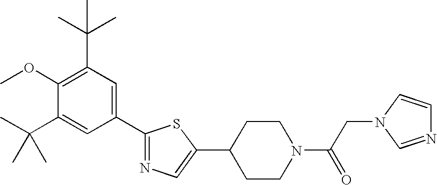

Using the method of Example 1, Step L with the product of Example 1, Step H and [1H-imidazol-1-yl]acetic acid as the starting materials the title compound was obtained. ¹H NMR (CD₃OD): δ 7.80 (s, 2H), 7.62 (s, 1H), 7.14 (s, 1H), 7.08 (s, 1H), 6.97 (s, 1H), 5.15 (½AB, 1H, J=16.9 Hz), 5.07 (½AB, 1H, J=17.1 Hz), 4.58 (bd, 1H, J=14.2 Hz), 4.04 (bd, 1H, J=13.9 Hz), 3.72 (s, 3H), 3.33 (bt, 1H, J=13.5 Hz), 3.12 (vbt, 1H, J=11.8 Hz), 2.88 (bt, 1H, J=12.8 Hz), 2.19 (bd, 1H, J=12.6 Hz), 2.12 (bd, 1H, J=12.0 Hz), 1.81 (dquart, 1H, J_{q}=12.6, J_{d}=3.3 Hz), 1.70 (dquart, 1H, J_{q}=12.6, J_{d}=4.0 Hz), 1.46 (s, 18H). LRMS calc: 494.3 obs: 495.4 (M+H).

### Example 36

### Step A methyl 3,5-dioxohexanoate

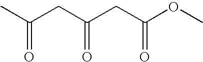

A solution of dehydroacetic acid (20.60 g; 168 mmol) in methanol (400 mL) was treated with a solution of magnesium methoxide (6 wt % in methanol; 350 mL; 184 mmol) at ambient temperature. The reaction was refluxed for 5 h. The solvent was removed and the residue added to aq. HCl (1 L; 1N). The aqueous was extracted (EtOAc, 2×500 mL). The organic was dried over MgSO₄, filtered and evaporated to give the title compound (17.0 g).

### Step B (5-methyl-1H-pyrazol-3-yl)acetic acid methyl ester

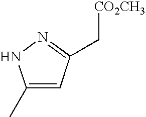

A solution of the product of Example 36, Step A (6.0 g; 38 mmol) in ethanol (40 mL) was treated dropwise with hydrazine monohydrate (2.21 mL; 46 mmol) at ambient temperature. The reaction was refluxed for 3 h. The solvent was removed and the residue chromatographed on silica gel (CH2Cl₂/acetone/acetic acid; 30:10:1) to give the title compound (2.3 g).

### Step C benzyl methyl 2,2′-(5-methyl-1H-pyrazole-1,3-diyl)diacetate

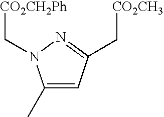

A solution of the product of Example 36, Step B (462 mg; 3.0 mmol) in dry DMF (8 mL) was treated with potassium carbonate (415 mg; 3.0 mmol) at ambient temperature. The mixture was warmed to 50° C. Benzyl 2-bromoacetate (687 mg; 3.0 mmol) was added dropwise. The reaction mixture was stirred for 4 h then cooled to ambient and stirred for 16 h. The mixture was diluted with water and extracted (EtOAc). The organic was dried over MgSO₄, filtered and evaporated to a residue. Silica gel chromatography (EtOAc/hexanes; 1:2) gave a mixture of two isomers. The mixture was chromatographed on Chiralcel OJ stationary phase (60% ethanol/heptane; λ=220 nM) to give the title compound (194 mg).

### Step D [3-(2-methoxy-2-oxoethyl)-5-methyl-1H-pyrazol-1-yl]acetic acid

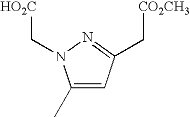

A solution of the product of Example 36, Step C (194 mg; 0.64 mmol) in methanol (50 mL) was treated with 10% palladium on carbon hydrogenation catalyst (60 mg). The mixture was shaken under a hydrogen atmosphere (1 atm) for 2 h. The mixture was filtered through Celite and the filtrate concentrated to give the title compound (118 mg).

### Step E [1-(2-{4-[2-(3,5-di-tert-butylphenyl)-1,3-thiazol-4-yl]piperidin-1-yl}-2-oxoethyl)-5-methyl-1H-pyrazol-3-yl]acetic acid methyl ester

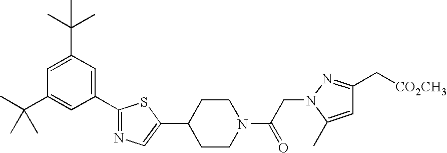

Using the method of Example 1, Step L with the products of Example 15, Step E and Example 36, Step D as the starting materials the title compound was obtained.

### Step F [1-(2-{4-[2-(3,5-di-tert-butylphenyl)-1,3-thiazol-4-yl]piperidin-1-yl}-2-oxoethyl)-5-methyl-1H-pyrazol-3-yl]acetic acid

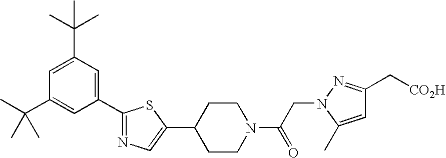

Using the method of Example 3, Step C with the product of Example 36, Step E as starting material the title compound was obtained. Isolation was effected by partitioning the reaction mixture between isopropyl acetate and pH4 phthalate buffer. The organic was dried over MgSO₄, filtered and evaporated. ¹H NMR (CD₃OD): δ 7.79 (d, 2H, J=1.4 Hz), 7.59 (bs, 1H), 7.22 (s, 1H), 6.10 (vbs, 1H), 5.16 (½AB, 1H, J=16.7 Hz), 5.08 (½AB, 1H, J=16.8 Hz), 4.62 (vbd, 1H, J=11.2 Hz), 4.14 (bd, 1H, J=14.0 Hz), 3.59 (vbs, 2H), 3.37 (bt, 1H, J=13.7 Hz), 3.18 (vbt, 1H, J=11.2), 2.93 (bt, 1H, J=13.2 Hz), 2.28 (s, 3H), 2.22 (bd, 1H, J=12.0 Hz), 2.16 (bd, 1H, J=12.3 Hz), 1.85 (vbquart, 1H, J=12.0 Hz), 1.73 (vbquart, 1H, J=12.1 Hz), 1.40 (s, 18H). LRMS calc: 536.3 obs: 537.3 (M+H).

### Step G (1-{2-[4-(2-{3-tert-butyl-5-[(trifluoromethyl)thio]phenyl}-1,3-thiazol-4-yl)piperidin-1-yl]-2-oxoethyl}-5-methyl-1H-pyrazol-3-yl)acetic acid methyl ester

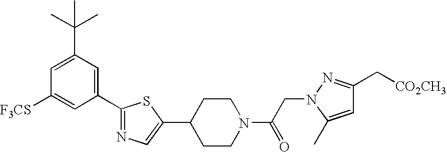

Using the method of Example 1, Step L with the product of Example 11, Step L and Example 36, Step D as starting materials the title compound was obtained.

### Step H Potassium (1-{2-[4-(2-{3-tert-butyl-5-[(trifluoromethyl)thio]phenyl}-1,3-thiazol-4-yl)piperidin-1-yl]-2-oxoethyl}-5-methyl-1H-pyrazol-3-yl)acetate

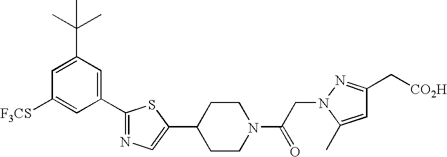

Using the method of Example 3, Step C with the product of Example 36, Step G as starting material the title compound was obtained. Isolation was effected by removal of volatiles and digestion of the derived solids in hot ethanol. The mixture was filtered and the filtrate evaporated to give the title compound. ¹H NMR (CD₃OD): δ 8.12 (bdd, 1H, J=5.1, 1.61 Hz), 8.03 (bd, 1H, J=4.4 Hz), 7.77 (bd, 1H, J=4.6 Hz), 7.28 (bd, 1H, J=5.8 Hz), 6.03 (bd, 1H, J=8.1 Hz), 5.07 (½AB, 1H, J=17.2 Hz), 5.00 (½AB, 1H, J=17.4 Hz), 4.58 (bd, 1H, J=13.3 Hz), 4.10 (vbs, 1H), 3.41 (vbd, 2H, J=5.5 Hz), 3.33 (bt, 1H, J=12.2 Hz), 3.15 (vbs, 1H), 2.88 (bt, 1H, J=12.7 Hz), 2.21-2.17 (s overlapping bd, 4H total), 2.12 (bd, 1H, J=12.9 Hz), 1.83 (vbquart, 1H, J=12.5 Hz), 1.72 (vbquart, 1H, J=12.5 Hz), 1.39 (s, 4.5H), 1.38 (s, 4.5H). LRMS calc: 580.2 obs: 581.3 (M+H).

### Example 37

### Step A 4-[2-(3,5-di-tert-butylphenyl)-1,3-thiazol-4-yl]-1-[(2,4-dimethyl-1H-imidazol-1-yl)acetyl]piperidine

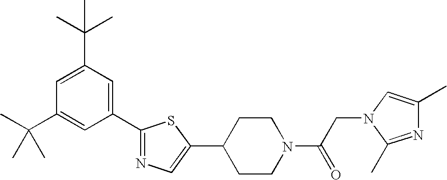

Using the method of Example 1, Step L with the products of Example 15, Step F and Example 2, Step B as starting materials the title compound was obtained. ¹H NMR (CDCl₃): δ 7.78 (bs, 2H), 7.54 (bs, 1H), 6.90 (s, 1H), 4.73 (bd, 1H, J=13.2 Hz), 4.73-4.63 (AB, 2H, J=16.9 Hz), 3.90 (bd, 1H, J=13.2 Hz), 3.32 (bt, 1H, J=12.1 Hz), 3.14 (bt, 1H, J=11.3 Hz), 2.89 (bt, 1H, J=11.8 Hz), 2.35 (bs, 3H), 2.28 (bd, 1H, J=13.7 Hz), 2.21-2.16 (bs overlapping bd, 4H total), 1.84-1.72 (bmult, 2H), 1.41 (s, 18H). LRMS calc: 492.3 obs: 493.4 (M+H).

### Step B 1-(2-{4-[2-(3,5-di-tert-butylphenyl)-1,3-thiazol-4-yl]piperidin-1-yl}-2-oxoethyl)-3-ethyl-1,3-dihydro-2H-imidazol-2-one

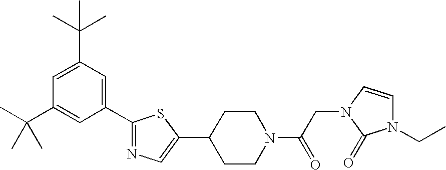

Using the method of Example 1, Step L with the products of Example 15, Step F and Example 4, Step C as starting materials the title compound was obtained. ¹H NMR (CD₃OD): δ 7.79 (d, 2H, J=1.8 Hz), 7.59 (t, 1H, J=1.7 Hz), 7.21 (s, 1H), 6.55 (d, 1H, J=2.8 Hz), 6.48 (d, 1H, J=2.8 Hz), 4.68 (½AB, 1H, J=16.7 Hz), 4.60 (½AB, 1H, J=16.6 Hz), 4.60 (bd, 1H, J=13.1 Hz), 4.09 (bd, 1H, J=13.5 Hz), 3.70 (quart, 2H, J=7.3 Hz), 3.31 (bt, 1H, J=12.0 Hz), 3.15 (tt, 1H, J=11.9, 3.4 Hz), 2.90 (dt, 1H, Jₜ=13.0, J_{d}=2.5 Hz), 2.22 (bd, 1H, J=13.0 Hz), 2.16 (bd, 1H, J=12.8 Hz), 1.85 (dquart, 1H, J_{q}=12.8, J_{d}=3.6 Hz), 1.74 (dquart, 1H, J_{q}=12.6, J_{d}=3.7 Hz), 1.40 (s, 18H), 1.30 (t, 3H, J=7.3 Hz). LRMS calc: 508.3 obs: 509.3 (M+H).

### Example 38

### Step A 3,5-bis(trifluoromethyl)benzoyl chloride

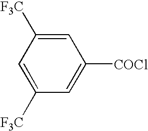

Using the method of Example 1, Step A with 3,5-bis(trifluoromethyl)benzoic acid as the starting material the title compound was obtained.

### Step B 3,5-bis(trifluoromethyl)benzamide

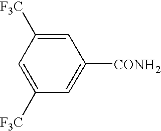

Using the method of Example 13, Step F with the product of Example 38, Step A as the starting material the title compound was obtained.

### Step C 3,5-bis(trifluoromethyl)benzenecarbothioamide

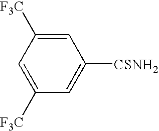

Using the method of Example 1, Step F with the product of Example 38, Step B as the starting material the title compound was obtained.

### Step D 9H-fluoren-9-ylmethyl4-{2-[3,5-bis(trifluoromethyl)phenyl]-1,3-thiazol-4-yl}piperidine-1-carboxylate

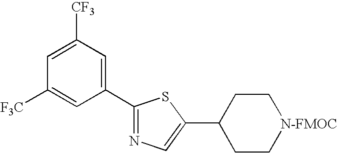

Using the method of Example 1, Step G with the products of Example 1, Step C and Example 38, Step C as the starting materials the title compound was obtained.

### Step E 4-{2-[3,5-bis(trifluoromethyl)phenyl]-1,3-thiazol-4-yl}piperidine

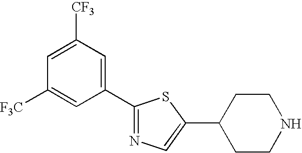

Using the method of Example 1, Step H with the product of Example 38, Step D as starting material the title compound was obtained.

### Step F 3-[2-(4-{2-[3,5-bis(trifluoromethyl)phenyl]-1,3-thiazol-4-yl}piperidin-1-yl)-2-oxoethyl]-3H-imidazo[4,5-b]pyridine

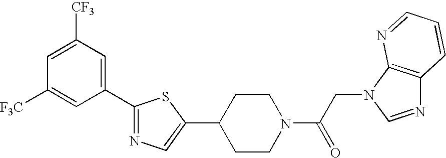

Using the method of Example 1, Step L with the products of Example 38, Step E and Example 1, Step K as the starting materials the title compound was obtained. ¹H NMR (CDCl₃): δ 8.42 (d, 1H, J=4.1 Hz), 8.37 (s, 2H), 8.13 (d, 1H, J=7.4 Hz), 7.92 (s, 1H), 7.30 (bmult, 2H), 7.07 (s, 1H), 5.31-5.23 (vbmult, 2H), 4.70 (bd, 1H, J=13.5 Hz), 4.15 (bd, 1H, J=13.7 Hz), 3.40 (bt, 1H, J=12.6 Hz), 3.14 (bt, 1H, J=11.6 Hz), 2.89 (bt, 1H, J=12.8 Hz), 2.28 (bd, 1H, J=13.0 Hz), 2.17 (bd, 1H, J=12.9 Hz), 1.88 (bquart, 1H, J=12.5 Hz), 1.79 (bdquart, 1H, J_{q}=12.5, J_{d}=3.6 Hz). LRMS calc: 539.1 obs: 540.1 (M+H).

### Step G 4-{2-[3,5-bis(trifluoromethyl)phenyl]-1,3-thiazol-4-yl}-1-[(3,5-dimethyl-1H-1,2,4-triazol-1-yl)acetyl]piperidine

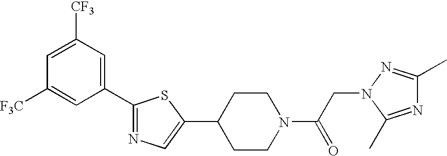

Using the method of Example 1, Step L with the products of Example 38, Step E and (3,5-dimethyl-1H-1,2,4-triazol-1-yl)acetic acid as the starting materials the title compound was obtained. ¹H NMR (CD₃OD): δ 8.50 (bs, 2H), 8.08 (bs, 1H), 7.45 (s, 1H), 5.23 (½AB, 1H, J=16.9 Hz), 5.16 (½AB, 1H, J=17.0 Hz), 4.60 (bd, 1H, J=13.0 Hz), 4.11 (bd, 1H, J=13.5 Hz), 3.40 (bt, 1H, J=12.9 Hz), 3.23 (vbt, 1H, J=11.7 Hz), 2.95 (bt, 1H, J=12.7 Hz), 2.40 (d, 3H, J=1.1 Hz), 2.30 (d, 3H, J=1.1 Hz), 2.25 (bd, 1H, J=13.2 Hz), 2.17 (bd, 1H, J=13.0 Hz), 1.94 (dquart, 1H, J_{q}=12.4, J_{d}=2.8 Hz), 1.79 (dquart, 1H, J_{q}=12.5, J_{d}=3.2 Hz). LRMS calc: 517.1 obs: 518.2 (M+H).

### Step H 4-{2-[3,5-bis(trifluoromethyl)phenyl]-1,3-thiazol-4-yl}-1-(chloroacetyl)piperidine

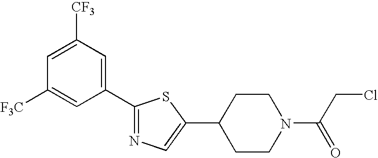

Using the method of Example 15, Step F with the product of Example 38, Step E as the starting material the title compound was obtained.

### Step J 3-[2-(4-{2-[3,5-bis(trifluoromethyl)phenyl]-1,3-thiazol-4-yl}piperidin-1-yl)-2-oxoethyl]-3H-imidazo[4,5-c]pyridine and 1-[2-(4-{2-[3,5-bis(trifluoromethyl)phenyl]-1,3-thiazol-4-yl}piperidin-1-yl)-2-oxoethyl]-1H-imidazo[4,5-c]pyridine

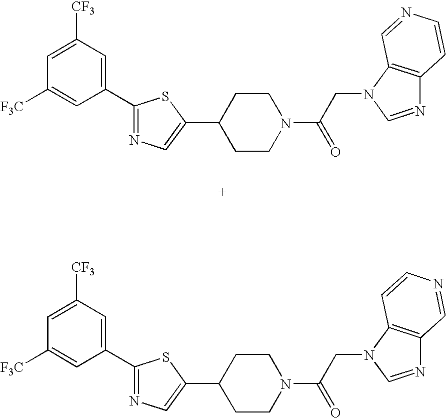

Using the method of Example 15, Step G with the product of Example 38, Step H as starting material the title compounds were obtained. ¹H NMR (CD₃OD): δ 8.88 (s, 1H), 8.45 (bs, 2H) 8.33 (s, 1H), 8.31 (d, 1H, J=4.0 Hz), 8.13 (bs, 1H), 7.77 (d, 1H, J=5.8 Hz), 7.19 (s, 1H), 5.67 (½AB, 1H, J=17.0 Hz), 5.59 (½AB, 1H, J=16.9 Hz), 4.60 (bd, 1H, J=13.0 Hz), 4.13 (bd, 1H, J=13.0 Hz), 3.43 (dt, 1H, Jₜ=13.3, J_{d}=2.1 Hz), 3.13 (tt, 1H, J=11.8, 3.5 Hz), 2.91 (dt, 1H, Jₜ=12.6, J_{d}=2.2 Hz), 2.26 (bd, 1H, J=13.0 Hz), 2.15 (bd, 1H, J=13.0 Hz), 1.90 (dquart, 1H, J_{q}=12.9, J_{d}=4.0 Hz), 1.79 (dquart, 1H, J_{q}=12.7, J_{d}=4.1 Hz), 1.41 (s, 18H). LRMS calc: 539.1 obs: 540.2. ¹H NMR (CD₃OD): δ 8.91 (s, 1H), 8.41 (bs, 2H), 8.36 (d, 1H, J=5.5 Hz), 8.30 (s, 1H), 8.17 (bs, 1H), 7.69 (d, 1H, J=5.6 Hz), 7.20 (s, 1H), 5.55 (½AB, 1H, J=17.1 Hz), 5.47 (½AB, 1H, J=17.2 Hz), 4.59 (bd, 1H, J=12.9 Hz), 4.14 (bd, 1H, J=13.0 Hz), 3.45 (dt, 1H, Jₜ=13.0, J_{d}=2.3 Hz), 3.12 (tt, 1H, J=11.9, 3.3 Hz), 2.89 (bt, 1H, J=12.6 Hz), 2.19 (bd, 1H, J=12.8 Hz), 2.14 (bd, 1H, J=12.7 Hz), 1.95 (dquart, 1H, J_{q}=12.7, J_{d}=3.5 Hz), 1.77 (dquart, 1H, J_{q}=12.8, J_{d}=3.8 Hz), 1.37 (s, 18H). LRMS calc: 539.1 obs: 540.1 (M+H).

### Step K 1-[2-(4-{2-[3,5-bis(trifluoromethyl)phenyl]-1,3-thiazol-4-yl}piperidin-1-yl)-2-oxoethyl]-2-methyl-1H-imidazo[4,5-c]pyridine

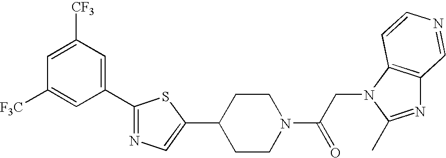

Using the method of Example 15, Step G with the product of Example 38, Step H and 2-methyl-5-azabenzimidazole as starting materials the title compound was obtained. ¹H NMR (CD₃OD): δ 8.80 (s, 1H), 8.48 (s, 1H), 8.30 (d, 1H, J=5.6 Hz), 8.05 (s, 1H), 7.55 (d, 1H, J=5.7 Hz), 7.44 (s, 1H), 5.41 (½AB, 1H, J=17.6 Hz), 5.34 (½AB, 1H, J=17.5 Hz), 4.56 (vbs, 1H), 4.20 (bd, 1H, J=7.1 Hz), 3.46 (bt, 1H, J=11.7 Hz), 3.23 (tt, 1H, J=11.8, 3.7 Hz), 2.95 (bt, 1H,J=11.7 Hz), 2.58 (s, 3H), 2.29 (bd, 1H,J=14.0 Hz), 2.15 (bd, 1H, J=13.8 Hz), 2.00 (dquart, 1H, J_{q}=12.0, J_{d}=2.4 Hz), 1.79 (dquart, 1H, J_{q}=12.2, J_{d}=3.8 Hz). LRMS calc: 553.1 obs: 554.1 (M+H).

### Example 39

### Step A 1-(2-tert-butoxy-2-oxoethyl)-1H-imidazole-2-carboxylic acid ethyl ester

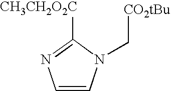

Using the method of Example 4, Step B with 1H-imidazole-2-carboxylic acid ethyl ester as the starting material the title compound was obtained.

### Step B [2-(ethoxycarbonyl)-1H-imidazol-1-yl]acetic acid hydrotrifluoroacetate

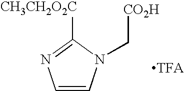

Using the method of Example 2, Step B with the product of Example 39, Step A as the starting material the title compound was obtained.

### Step C 1-(2-{4-[2-(3,5-di-tert-butyl-4-methoxyphenyl)-1,3-thiazol-4-yl]piperidin-1-yl}-2-oxoethyl)-1H-imidazole-2-carboxylic acid ethyl ester

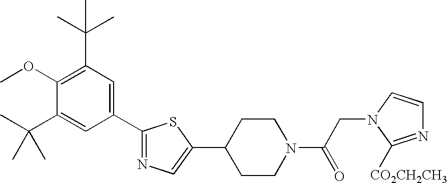

Using the method of Example 1, Step L with the products of Example 1, Step H and Example 39, Step B as the starting materials the title compound was obtained.

### Step D 1-(2-{4-[2-(3,5-di-tert-butyl-4-methoxyphenyl)-1,3-thiazol-4-yl]piperidin-1-yl}-2-oxoethyl)-1H-imidazole-2-carboxylic acid dihydrochloride

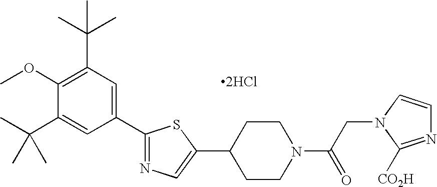

A solution of the product of Example 39, Step C (21 mg; 0.038 mmol) in ethanol (1 mL) was treated with aq. potassium hydroxide (76 μL; 1M; 0.076 mmol). The solution was stirred at ambient temperature for 48 h. The ethanol was evaporated and the remaining residue dissolved in methanol (0.5 mL) and diluted with aq. HCl (10 mL; 1N). Lyophilization afforded a solid that was digested in hot ethanol (3 mL). The mixture was filtered and the filtrate evaporated to give the title compound (20 mg). ¹H NMR (CD₃OD): δ 7.95 (s, 2H), 7.87 (bs, 1H), 7.78 (s, 1H), 7.63 (s, 1H), 5.78 (½AB, 1H, J=15.5 Hz), 5.67 (½AB, 1H, J=15.7 Hz), 4.62 (bd, 1H, J=12.6 Hz), 4.12 (bd, 1H, J=11.9 Hz), 3.79 (s, 3H), 3.47 (vbt, 1H, J=11.0 Hz), 3.39 (vbs, 1H), 2.99 (bt, 1H, J=12.2 Hz), 2.32 (bd, 1H, J=10.0 Hz), 2.23 (bd, 1H, J=11.5 Hz), 2.01 (vbd, 1H, J=9.6 Hz), 1.77 (vbquart, 1H, J=10.3 Hz), 1.52 (s, 18H). LRMS calc: 538.3 obs: 539.4 (M+H).

### Example 40

### Step A 9H-fluoren-9-ylmethyl 4-(2-phenyl-1,3-thiazol-4-yl)piperidine-1-carboxylate

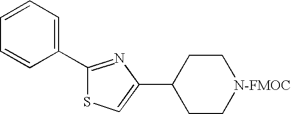

Using the method of Example 1, Step G with the product of Example 1, Step C and benzenecarbothioamide as the starting materials the title compound was obtained.

### Step B 4-(2-phenyl-1,3-thiazol-4-yl)piperidine

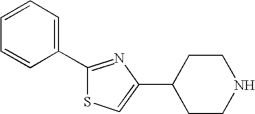

Using the method of Example 1, Step H with the product of Example 40, Step A as starting material the title compound was obtained.

### Step C 1-ethyl-3-{2-oxo-2-[4-(2-phenyl-1,3-thiazol-4-yl)piperidin-1-yl]ethyl-1,3-dihydro-2H-imidazol-2-one

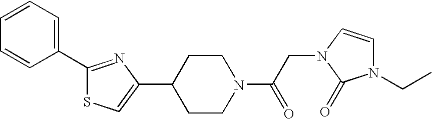

Using the method of Example 1, Step L with the products of Example 40, Step B and Example 4, Step C as the starting materials the title compound was obtained. ¹H NMR (CD₃OD): δ 7.91 (mutt, 2H), 7.45 (mult, 3H), 7.19 (s, 1H), 6.51 (d, 1H, J=2.8 Hz), 6.44 (d, 1H, J=2.9 Hz), 4.64 (½AB, 1H, J=16.7 Hz), 4.56 (½AB overlapping bs, 2H total, J_{AB}=16.8 Hz), 4.04 (bd, 1H, J=13.0 Hz), 3.67 (quart, 2H, J=7.3 Hz), 3.31 (bt, 1H, J=12.0 Hz), 3.11 (bt, 1H, J=12.2 Hz), 2.87 (bt, 1H, J=12.0 Hz), 2.16 (bd, 1H, J=12.9 Hz), 2.10 (bd, 1H, J=12.9 Hz), 1.81 (dquart, 1H, J_{q}=12.6, J_{d}=3.7 Hz), 1.73 (dquart, 1H, J_{q}=12.6, J_{d}=3.8 Hz), 1.26 (t, 3H, J=7.2 Hz). LRMS calc: 396.2 obs: 397.2 (M+H).

### Step D 9H-fluoren-9-ylmethyl 4-[2-(4-methoxyphenyl)-1,3-thiazol-4-yl]piperidine-1-carboxylate

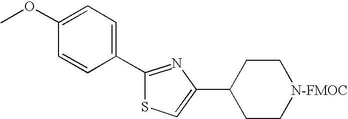

Using the method of Example 1, Step G with the product of Example 1, Step C and 4-methoxybenzenecarbothioamide as the starting materials the title compound was obtained.

### Step E 4-[2-(4-methoxyphenyl)-1,3-thiazol-4-yl]piperidine

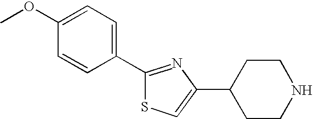

Using the method of Example 1, Step H with the product of Example 40, Step D as starting material the title compound was obtained.

### Step F 1-ethyl-3-(2-{4-[2-(4-methoxyphenyl-1,3-thiazol-4-yl]piperidin-1-yl}-2-oxoethyl)-1,3-dihydro-2H-imidazol-2-one

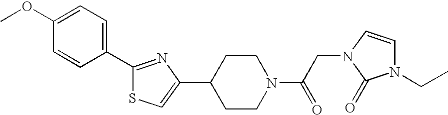

Using the method of Example 1, Step L with the products of Example 40, Step E and Example 4, Step C as the starting materials the title compound was obtained. ¹H NMR (CD₃OD): δ 7.84 (d, 2H, J=8.8 Hz), 7.09 (s, 1H), 6.99 (d, 2H, J=8.8 Hz), 6.51 (d, 1H, J=2.8 Hz), 6.44 (d, 1H, J=2.9 Hz), 4.63 (½AB, 1H, J=16.6 Hz), 4.55 (½AB overlapping bs, 2H total, J_{AB}=16.6 Hz), 4.03 (bd, 1H, J=13.2 Hz), 3.83 (s, 3H), 3.67 (quart, 2H, J=7.3 Hz), 3.31 (bt, 1H, J=12.0 Hz), 3.08 (tt, 1H, J=11.7, 3.6 Hz), 2.86 (dt, 1H, Jₜ=12.7, J_{d}=2.1 Hz), 2.15 (bd, 1H, J=11.9 Hz), 2.09 (bd, 1H, J=12.0 Hz), 1.80 (dpent, 1H, Jₚ=12.2, J_{d}=3.5 Hz), 1.69 (dpent, 1H, Jₚ=12.4, J_{d}=3.6 Hz), 1.26 (t, 3H, J=7.2 Hz). LRMS calc: 426.2 obs: 427.3 (M+H).

### Step G 9H-fluoren-9-ylmethyl 4-[2-(4-tert-butylphenyl)-1,3-thiazol-4-yl]piperidine-1-carboxylate

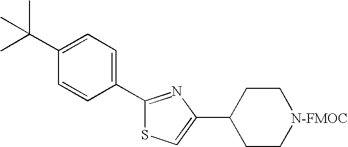

Using the method of Example 1, Step G with the product of Example 1, Step C and 4-tert-butylbenzenecarbothioamide as the starting materials the title compound was obtained.

### Step H 4-[2-(4-tert-butylphenyl)-1,3-thiazol-4-yl]piperidine

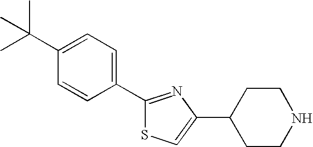

Using the method of Example 1, Step H with the product of Example 40, Step G as starting material the title compound was obtained.

### Step J 1-(2-{4-[2-(4-tert-butylphenyl-1,3-thiazol-4-yl]piperidin-1-yl}-2-oxoethyl)-3-ethyl-1,3-dihydro-2H-imidazol-2-one

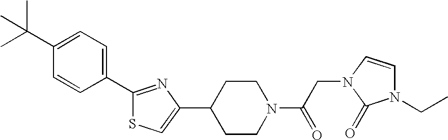

Using the method of Example 1, Step L with the products of Example 40, Step H and Example 4, Step C as the starting materials the title compound was obtained. ¹H NMR (CD₃OD): δ 7.83 (d, 2H, J=8.5 Hz), 7.49 (d, 2H, J=8.6 Hz), 7.15 (s, 1H), 6.51 (d, 1H, J=2.8 Hz), 6.44 (d, 1H, J=2.9 Hz), 4.63 (½AB, 1H, J=16.6 Hz), 4.56 (½AB overlapping bs, 2H total, J_{AB}=16.7 Hz), 4.04 (bd, 1H, J=12.7 Hz), 3.67 (quart, 2H, J=7.3 Hz), 3.33 (bt, 1H, J=12.0 Hz), 3.10 (tt, 1H, J=11.5, 3.5 Hz), 2.86 (bt, 1H, J=11.6 Hz), 2.15 (bd, 1H, J=13.0 Hz), 2.10 (bd, 1H, J=12.5 Hz), 1.80 (dquart, 1H, J_{q}=12.4, J_{d}=3.5 Hz), 1.69 (dquart, 1H, J_{q}=12.4, J_{d}=3.6 Hz), 1.34 (s, 9H), 1.26 (t, 3H, J=7.2 Hz). LRMS calc: 452.2 obs: 453.3 (M+H).

### Step K 9H-fluoren-9-ylmethyl 4-[2(4-chlorophenyl)-1,3-thiazol-4-yl]piperidine-1-carboxylate

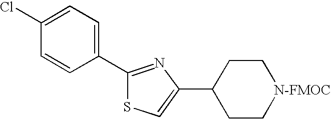

Using the method of Example 1, Step G with the product of Example 1, Step C and 4-chlorobenzenecarbothioamide as the starting materials the title compound was obtained.

### Step L 4-[2-(4-chlorophenyl)-1,3-thiazol-4-yl]piperidine

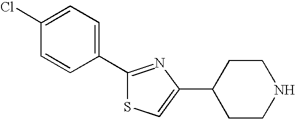

Using the method of Example 1, Step H with the product of Example 40, Step K as starting material the title compound was obtained.

### Step M 1-(2-{4-[2-(4-chlorophenyl)-1,3-thiazol-4-yl]piperidin-1-yl}-2-oxoethyl)-3-ethyl-1,3-dihydro-2H-imidazol-2-one

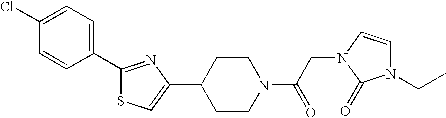

Using the method of Example 1, Step L with the products of Example 40, Step L and Example 4, Step C as the starting materials the title compound was obtained. ¹H NMR (CD₃OD): δ 7.91 (d, 2H, J=8.5 Hz), 7.46 (d, 2H, J=8.6 Hz), 7.22 (s, 1H), 6.51 (d, 1H, J=2.8 Hz), 6.44 (d, 1H, J=2.9 Hz), 4.63 (½AB, 1H, J=16.8 Hz), 4.56 (½AB overlapping bs, 2H total, J_{AB}=16.8 Hz), 4.04 (bd, 1H, J=13.9 Hz), 3.67 (quart, 2H, J=7.3 Hz), 3.31 (bt, 1H, J=11.8 Hz), 3.11 (tt, 1H, J=11.6, 4.0 Hz), 2.87 (dt, 1H, Jₜ=2.2 Hz), 2.15 (bd, 1H, J=13.0 Hz), 2.09 (bd, 1H, J=13.1 Hz), 1.81 (dquart, 1H, J_{q}=12.6, J_{d}=3.4 Hz), 1.70 (dquart, 1H, J_{q}=12.3, J_{d}=3.7 Hz), 1.26 (t, 3H, J=7.2 Hz). LRMS calc: 430.1 obs: 431.2 (M+H).

### Step N 9H-fluoren-9-ylmethyl 4-[2-(4-bromophenyl)-1,3-thiazol-4-yl]piperidine-1-carboxylate

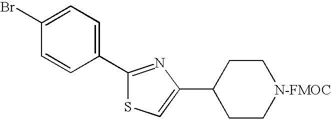

Using the method of Example 1, Step G with the product of Example 1, Step C and 4-bromobenzenecarbothioamide as the starting materials the title compound was obtained.

### Step P 4-[2-(4-bromophenyl)-1,3-thiazol-4-yl]piperidine

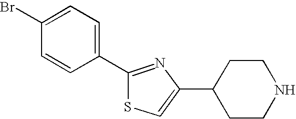

Using the method of Example 1, Step H with the product of Example 40, Step N as starting material the title compound was obtained.

### Step Q 1-(2-{4-[2-(4-bromophenyl)-1,3-thiazol-4-yl]piperidin-1-yl}-2-oxoethyl)-3-ethyl-1,3-dihydro-2H-imidazol-2-one

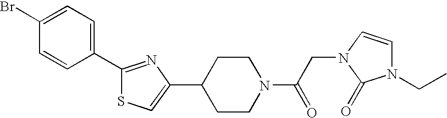

Using the method of Example 1, Step L with the products of Example 40, Step P and Example 4, Step C as the starting materials the title compound was obtained. ¹H NMR (CD₃OD): δ 7.89 (d, 2H, J=8.4 Hz), 7.66 (d, 2H, J=8.5 Hz), 7.26 (s, 1H), 6.54 (d, 1H, J=2.9 Hz), 6.47 (d, 1H, J=3.0 Hz), 4.68 (½AB, 1H, J=16.7 Hz), 4.59 (½AB overlapping bs, 2H total, J_{AB}=16.6 Hz), 4.07 (bd, 1H, J=13.0 Hz), 3.70 (quart, 2H, J=7.3 Hz), 3.31 (bt, 1H, J=11.8 Hz), 3.15 (tt, 1H, J=11.5, 3.6 Hz), 2.90 (dt, 1H, Jₜ=13.0, J_{d}=2.1 Hz), 2.19 (bd, 1H, J=12.6 Hz), 2.13 (bd, 1H, J=12.6 Hz), 1.85 (dquart, 1H, J_{q}=12.4, J_{d}=3.7 Hz), 1.74 (dquart, 1H, J_{q}=12.3, J_{d}=3.9 Hz), 1.30 (t, 3H, J=7.2 Hz). LRMS calc: 474.1 obs: 475.2 (M+H).

### Step R 9H-fluoren-9-ylmethyl 4-[2-(4-trifluoromethylphenyl)-1,3-thiazol-4-yl]piperidine-1-carboxylate

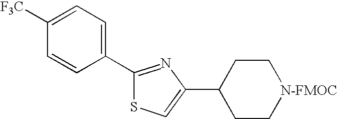

Using the method of Example 1, Step G with the product of Example 1, Step C and 4-trifluoromethylbenzenecarbothioamide as the starting materials the title compound was obtained.

### Step S 4-[2-(4-trifluoromethylphenyl)-1,3-thiazol-4-yl]piperidine

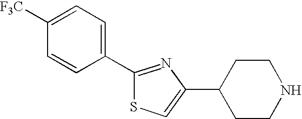

Using the method of Example 1, Step H with the product of Example 40, Step R as starting material the title compound was obtained.

### Step T 1-(2-{4-[2-(4-trifluoromethylphenyl)-1,3-thiazol-4-yl]piperidin-1-yl}-2-oxoethyl)-3-ethyl-1,3-dihydro-2H-imidazol-2-one

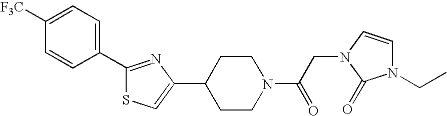

Using the method of Example 1, Step L with the products of Example 40, Step S and Example 4, Step C as the starting materials the title compound was obtained. ¹H NMR (CD₃OD): δ 8.11 (d, 2H, J=8.1 Hz), 7.76 (d, 2H, J=8.3 Hz), 7.31 (s, 1H), 6.51 (d, 1H, J=2.8 Hz), 6.44 (d, 1H, J=2.9 Hz), 4.64 (½AB, 1H, J=16.8 Hz), 4.56 (½AB overlapping bs, 2H total, J_{AB}=16.8 Hz), 4.05 (bd, 1H, J=13.0 Hz), 3.67 (quart, 2H, J=7.3 Hz), 3.33 (bt, 1H, J=12.2 Hz), 3.14 (tt, 1H, J=11.6, 3.4 Hz), 2.88 (bt, 1H, J=11.8 Hz), 2.17 (bd, 1H, J=12.8 Hz), 2.11 (bd, 1H, J=12.4 Hz), 1.83 (dquart, 1H, J_{q}=12.6, J_{d}=3.9 Hz), 1.73 (dquart, 1H, J_{q}=12.7, J_{d}=4.0 Hz), 1.26 (t, 3H, J=7.4 Hz). LRMS calc: 464.2 obs: 465.2 (M+H).

### Step U 9H-fluoren-9-ylmethyl 4-[2-(3-chlorophenyl)-1,3-thiazol-4-yl]piperidine-1-carboxylate

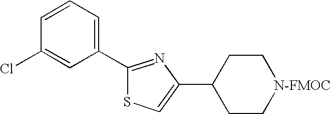

Using the method of Example 1, Step G with the product of Example 1, Step C and 3-chlorobenzenecarbothioamide as the starting materials the title compound was obtained.

### Step V 4-[2-(3-chlorophenyl)-1,3-thiazol-4-yl]piperidine

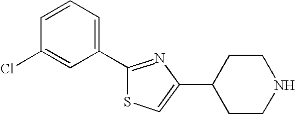

Using the method of Example 1, Step H with the product of Example 40, Step U as starting material the title compound was obtained.

### Step W 1-(2-{4-[2-(3-chlorophenyl)-1,3-thiazol-4-yl]piperidin-1-yl}-2-oxoethyl)-3-ethyl-1,3-dihydro-2H-imidazol-2-one

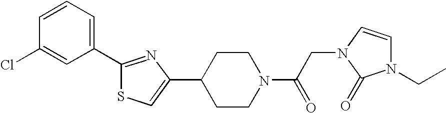

Using the method of Example 1, Step L with the products of Example 40, Step V and Example 4, Step C as the starting materials the title compound was obtained. ¹H NMR (CD₃OD): δ 7.94 (bs, 1H), 7.82-7.80 (bmult, 1H), 7.43-7.41 (bmult, 2H), 7.24 (s, 1H), 6.51 (d, 1H, J=2.8 Hz), 6.44 (d, 1H, J=2.9 Hz), 4.63 (½AB, 1H, J=16.8 Hz), 4.56 (½AB overlapping bs, 2H total, J_{AB}=16.8 Hz), 4.04 (bd, 1H, J=13.3 Hz), 3.67 (quart, 2H, J=7.4 Hz), 3.31 (bt, 1H, J=12.2 Hz), 3.11 (tt, 1H, J=11.5, 3.7 Hz), 2.86 (bt, 1H, J=11.6 Hz), 2.15 (bd, 1H, J=13.1 Hz), 2.09 (bd, 1H, J=13.2 Hz), 1.81 (dquart, 1H, J_{q}=12.3, J_{d}=3.8 Hz), 1.71 (dquart, 1H, J_{q}=12.2, J_{d}=3.8 Hz), 1.26 (t, 3H, J=7.2 Hz). LRMS calc: 430.1 obs: 431.2 (M+H).

### Step X 9H-fluoren-9-ylmethyl 4-[2(3-bromophenyl)-1,3-thiazol-4-yl]piperidine-1-carboxylate

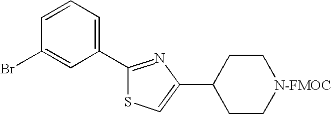

Using the method of Example 1, Step G with the product of Example 1, Step C and 3-bromobenzenecarbothioamide as the starting materials the title compound was obtained.

### Step Y 4-[2-(3-bromophenyl)-1,3-thiazol-4-yl]piperidine

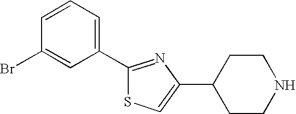

Using the method of Example 1, Step H with the product of Example 40, Step X as starting material the title compound was obtained.

### Step Z 1-[2-{4-[2-(3-bromophenyl)-1,3-thiazol-4-yl]piperidin-1-yl}-2-oxoethyl)-3-ethyl-1,3-dihydro-2H-imidazol-2-one

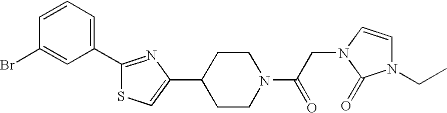

Using the method of Example 1, Step L with the products of Example 40, Step Y and Example 4, Step C as the starting materials the title compound was obtained. ¹H NMR (CD₃OD): δ 8.10 (bs, 1H), 7.86 (d, 1H, J=7.7 Hz), 7.58 (d, 1H, J=7.9 Hz), 7.37 (t, 1H, J=7.8 Hz), 7.25 (s, 1H), 6.51 (d, 1H, J=2.7 Hz), 6.44 (d, 1H, J=2.7 Hz), 4.64 (½AB, 1H, J=16.8 Hz), 4.56 (½AB overlapping bs, 2H total, J_{AB}=16.8 Hz), 4.04 (bd, 1H, J=12.3 Hz), 3.67 (quart, 2H, J=7.2 Hz), 3.33 (bt, 1H, J=12.5 Hz), 3.12 (bt, 1H, J=11.9 Hz), 2.87 (bt, 1H, J=11.9 Hz), 2.15 (bd, 1H, J=13.3 Hz), 2.09 (bd, 1H, J=13.0 Hz), 1.83 (dquart, 1H, J_{q}=12.7, J_{d}=3.4 Hz), 1.73 (dquart, 1H, J_{q}=12.5, J_{d}=3.6 Hz), 1.26 (t, 3H, J=7.3 Hz). LRMS talc: 474.1 obs: 475.1 (M+H).

### Step AA 9H-fluoren-9-ylmethyl 4-[2-(3-trifluoromethylphenyl)-1,3-thiazol-4-yl]piperidine-1-carboxylate

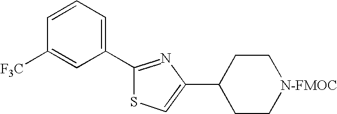

Using the method of Example 1, Step G with the product of Example 1, Step C and 3-trifluoromethylbenzenecarbothioamide as the starting materials the title compound was obtained.

### Step BB 4-[2-(3-trifluoromethylphenyl)-1,3-thiazol-4-yl]piperidine

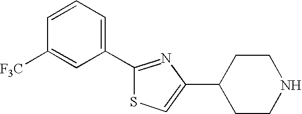

Using the method of Example 1, Step H with the product of Example 40, Step AA as starting material the title compound was obtained.

### Step CC 1-(2-{4-[2-(3-trifluoromethylphenyl)-1,3-thiazol-4-yl]piperidin-1-yl}-2-oxoethyl)-3-ethyl-1,3-dihydro-2H-imidazol-2-one

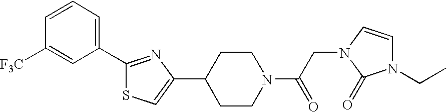

Using the method of Example 1, Step L with the products of Example 40, Step BB and Example 4, Step C as the starting materials the title compound was obtained. ¹H NMR (CD₃OD): δ 8.22 (bs, 1H), 8.15 (d, 1H, J=7.6 Hz), 7.73 (d, 1H, J=7.7 Hz), 7.66 (t, 1H, J=7.8 Hz), 7.29 (s, 1H), 6.51 (d, 1H, J=2.8 Hz), 6.44 (d, 1H, J=2.7 Hz), 4.64 (½AB, 1H, J=16.8 Hz), 4.56 (½AB overlapping bs, 2H total, J_{AB}=16.8 Hz), 4.05 (bd, 1H, J=13.0 Hz), 3.67 (quart, 2H, J=7.2 Hz), 3.33 (bt, 1H, J=13.0 Hz), 3.14 (tt, 1H, J=11.8, 3.6 Hz), 2.87 (dt, 1H, Jₜ=12.8, J_{d}=2.0 Hz), 2.17 (bd, 1H, J=13.2 Hz), 2.11 (bd, 1H, J=13.3 Hz), 1.84 (dquart, 1H, J_{q}=12.7, J_{d}=3.7 Hz), 1.72 (dquart, 1H, J_{q}=12.3, J_{d}=3.8 Hz), 1.26 (t, 3H, J=7.4 Hz). LRMS calc: 464.2 obs: 465.2 (M+H).

### Step DD 9H-fluoren-9-ylmethyl 4-[2-(3,5-dichlorophenyl)-1,3-thiazol-4-yl]piperidine-1-carboxylate

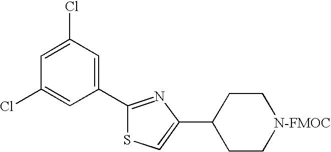

Using the method of Example 1, Step G with the product of Example 1, Step C and 3,5-dichlorobenzenecarbothioamide as the starting materials the title compound was obtained.

### Step EE 4-[2-(3,5-dichlorophenyl)-1,3-thiazol-4-yl]piperidine

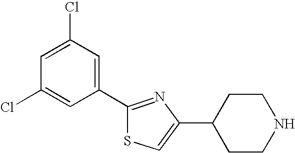

Using the method of Example 1, Step H with the product of Example 40, Step DD as starting material the title compound was obtained.

### Step FF 1(2-{4-[2-(3,5-dichlorophenyl)-1,3-thiazol-4-yl]piperidin-1-yl)-2-oxoethyl)-3-ethyl-1,3-dihydro-2H-imidazol-2-one

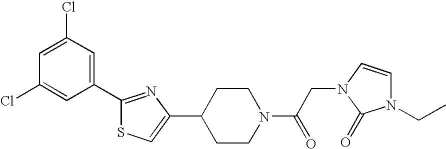

Using the method of Example 1, Step L with the products of Example 40, Step EE and Example 4, Step C as the starting materials the title compound was obtained. ¹H NMR (CD₃OD): δ 7.87 (d, 1H, J=1.6 Hz), 7.51 (d, 1H, J=1.6 Hz), 7.31 (s, 1H), 6.51 (d, 1H, J=2.8 Hz), 6.44 (d, 1H, J=2.8 Hz), 4.64 (½AB, 1H, J=16.4 Hz), 4.56 (½AB overlapping bs, 2H total, J_{AB}=16.5 Hz), 4.05 (bd, 1H, J=13.3 Hz), 3.67 (quart, 2H, J=7.4 Hz), 3.30 (bt, 1H, J=12.0 Hz), 3.12 (tt, 1H, J=11.5, 3.4 Hz), 2.87 (dt, 1H, Jₜ=12.6, J_{d}=2.2 Hz), 2.15 (bd, 1H, J=12.5 Hz), 2.09 (bd, 1H, J=12.7 Hz), 1.83 (dquart, 1H, J_{q}=12.4, J_{d}=3.7 Hz), 1.71 (dquart, 1H, J_{q}=12.5, J_{d}=3.8 Hz), 1.26 (t, 3H, J=7.2 Hz). LRMS calc: 464.1 obs: 465.2 (M+H).

### Step NN 9H-fluoren-9-ylmethyl 4(2-pyridin-4-yl-1,3-thiazol-4-yl)piperidine-1-carboxylate

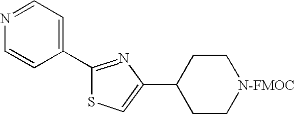

Using the method of Example 1, Step G with the product of Example 1, Step C and 4-pyridin-4-ylbenzenecarbothioamide as the starting materials the title compound was obtained.

### Step PP 4-(4-piperidin-4-yl-1,3-thiazol-2-yl)pyridine

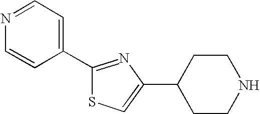

Using the method of Example 1, Step H with the product of Example 40, Step NN as starting material the title compound was obtained.

### Step QQ 1-ethyl-3-{2-oxo-2-[4-(2-pyridin-4-yl-1,3-thiazol-4-yl)piperidin-1-yl]ethyl}-1,3-dihydro-2H-imidazol-2-one

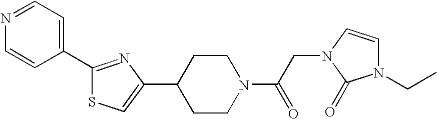

Using the method of Example 1, Step L with the products of Example 40, Step PP and Example 4, Step C as the starting materials the title compound was obtained. ¹H NMR (CD₃OD): δ 8.63 (dd, 2H, J=4.7, 1.5 Hz), 7.94 (dd, 2H, J=4.8, 1.5 Hz), 7.42 (s, 1H), 6.52 (d, 1H, J=2.9 Hz), 6.44 (d, 1H, J=2.9 Hz), 4.65 (½AB, 1H, J=16.8 Hz), 4.56 (½AB overlapping bs, 2H total, J_{AB}=16.7 Hz), 4.05 (bd, 1H, J=13.8 Hz), 3.67 (quart, 2H, J=7.3 Hz), 3.31 (bt, 1H, J=11.9 Hz), 3.16 (bt, 1H, J=11.7 Hz), 2.88 (dt, 1H, Jₜ=12.6, J_{d}=2.2 Hz), 2.17 (bd, 1H, J=11.5 Hz), 2.11 (bd, 1H, J=13.1 Hz), 1.84 (dquart, 1H, J_{q}=12.7, J_{d}=3.8 Hz), 1.73 (dquart, 1H, J_{q}=12.4, J_{d}=3.9 Hz), 1.26 (t, 3H, J=7.5 Hz). LRMS calc: 397.2 obs: 398.2 (M+H).

### Assays for Determining Biological Activity

### Human CXCR3 Receptor.

The compounds claimed here are assayed for affinity and functional potency at the CXCR3 receptor using the assays described below.

Since the expression of CXCR3 on naive T cells is low, PBMC's were cultured in the presence of a mixture of superantigens to provide primary cells with sufficient CXCR3 expression to use routinely in binding and functional assays. Briefly, mononuclear cells were enriched from buffy coats obtained from a local blood bank by centrifugation over Ficoll-Hypaque. Residual red blood cells were lysed in hypotonic buffer, (ACK), cells were washed with PBS and resuspended in media (RPMI containing 10% FBS, 2 mM glutamine, MEM non essential amino acids and sodium pyruvate) containing 500 Units/ml of IL-2 and 0.5 ng/ml SE cocktail (containing equal amounts of SEA, SEB, SEC1, SED and SEE all from Toxin Technology). After several days in culture, cells were switched to fresh media containing 500 units/ml of IL-2 and cultures were maintained at 2-4 million cells/ml for up to 21 days.

### Binding Assay.

Inhibition of binding of CXCL10 or CXCL11 to human CXCR3 was measured in whole cells, using superantigen activated T cells (SE-T) at day 7-14 post stimulation. Binding of ¹²⁵I-IP-10 (2200 Ci/mmol, typically 20 pM) in the presence of unlabeled ligands was initiated by adding intact T cells (200,000 cells/assay) in a total assay volume of 250 μl containing 50 mM HEPES, pH 7.2, 5 mM MgCl2, 1 mM CaCl2 and 0.5% BSA. Binding of ¹²⁵I-I-TAC (2200 Ci/mmol, 20 pM) was performed as described for IP-10 except for the addition of 0.15M NaCl to the binding buffer. After incubation at room temperature for 2 hours with shaking, the reaction was terminated by filtering through a 0.1% polyethylenimine (Sigma) soaked GF/C filter plate (Packard) using a Packard Filtermate cell harvester and the plate washed with approximately 750 μl of 50 mM HEPES (Sigma), pH 7.2, 500 mM NaCl chilled to 4° C. The plates were dried; scintillant added and counted on a Packard TopCount. Non-specific binding was measured in the presence of 1 μM ligand (IP-10 or I-TAC). Binding results were analyzed using Microsoft Excel and GraphPad Prism software.

The Examples disclosed herein were tested in the above receptor binding assay and demonstrated an IC₅₀ ranging from 1 to 1200 nM against ¹²⁵I-IP-10. The range of IC₅₀ values is similar against ¹²⁵I-I-TAC.

### Functional Assays.

The functional potency of the claimed compounds was assessed by measuring inhibition of the chemotaxis of leukocytes in response to CXCR3 ligands. A modified Boyden chamber chemotaxis system (ChemoTx™, NeuroProbe, Gaithersburg, Md.), consisting of a 96-well microplate and a filter (6.0-mm diameter, 5-μ pore size), coated on the bottom with fibronectin (50 μl of a 10 μg/ml solution, then air-dried), was used for chemotaxis measurements. Briefly, aliquots of human T cells (day 14 to day 17 post activation) were washed and resuspended at 1×107 cells/ml in warm (37° C.) Hanks' balanced saline solution (HBSS)/bovine serum albumin [(BSA); HBSS without phenol red, calcium, or magnesium (Mediatec)+0.01% BSA] and loaded with Calcein-AM (Molecular Probes) at a concentration of 2 μM for 30 min at 37° C. The cells were washed again in HBSS/BSA and resuspended in RPMI/BSA [RPMI without phenol red (Mediatec)+0.5% BSA+1% dimethyl sulfoxide] to a concentration of 6×106 cells/ml. To initiate the chemotaxis, chemokines were diluted in warm (37° C.) RPMI/BSA and added in 30 μl to the bottom of the microplate before affixing the filter to the unit. Aliquots (50 μl) of the Calcein-loaded T cells were then added to the top of the filter over each individual well. The microplates were subsequently incubated for 1 h at 37° C. Remaining cells were suctioned off the top of the filter. The filter was rinsed with PBS and wiped with a rubber squeegee. The plate with filter intact was read in a Cytofluor™ II fluorometer (PerSeptive Biosystems, Foster City, Calif.). For assay of antagonists, compounds were diluted in DMSO and added to both cells and ligand in a final DMSO concentration of 0.5%.

The Examples disclosed herein were tested in the above assay against both IP-10 and I-TAC. The Examples demonstrated an IC₅₀ ranging from 0.5 to 600 nM against IP-10 and typically a somewhat higher IC₅₀ ranging from 25 to 1700 nM against I-TAC.

What is claimed is:

1. A compound of Formula I 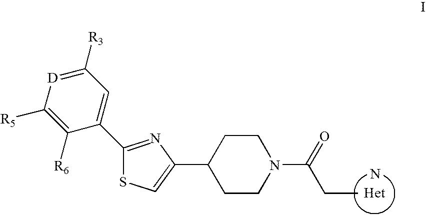
or a pharmaceutically acceptable salt thereof, wherein:
D is CR₄ or N;
R₃ is selected from the group consisting of: H, halo, C₁₋₄alkyl, —CF₃, —OCF₃ and —S(O)ₙCF₃, wherein n is 0 or 2;
R₄ is selected from the group consisting of: H, halo, —OH, C₁₋₄alkyl, —OCH₃, —OCH₂CF₃ and —CF₃;
or R₃ and R₄ may be joined together with the carbon atoms to which they are attached to form a five- or six-membered monocyclic ring, said rings containing oxygen or tetra-substituted with methyl groups as follows: 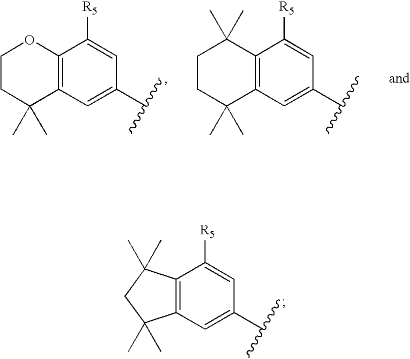
R₅ is selected from the group consisting of: —H, halo, C₁₋₄alkyl, C₃₋₆cycloalkyl, CF₃, —CF₂CH₃, —OCF₃ and —SCF₃;
R₆ is selected from the group consisting of —H and —OCH₃,
or R₅ and R₆ may be joined together with the carbon atoms to which they are attached to form a monocyclic 5-membered ring, said ring di-substituted with methyl as follows: 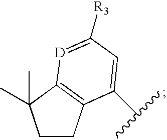
and 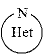
is a 5 membered non-aromatic or aromatic ring or a 9 membered fused bicyclic partially aromatic or aromatic ring, each ring containing at least 1 nitrogen atom and optionally up to 3 additional heterotaoms selected from S, O and N, said rings optionally substituted with 1 to 3 substituents independently selected from the group consisting of: oxo, hydroxy, carboxy, —CF₃, halo, —S(O)ₚ—CH₃, phenyl, C₁₋₃alkoxy and C₁₋₃alkyl, said C₁₋₃alkyl optionally substituted with carboxy or hydroxy; and
p is 0, 1 or 2.

2. The compound according to claim 1 wherein: 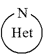
is selected from the group consisting of: 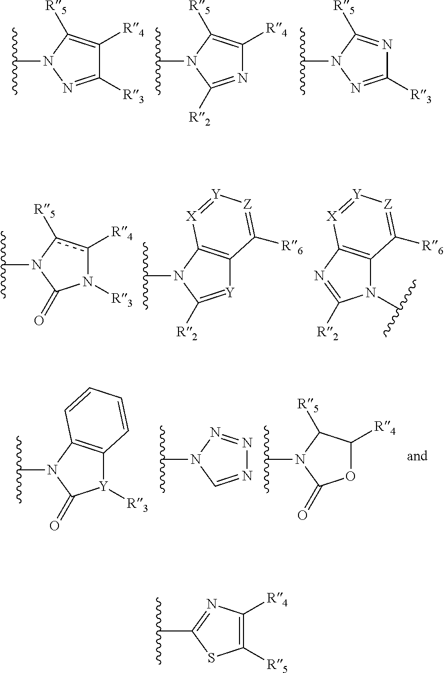
wherein X, Y and Z are independently C or N,
R″₂, R″₃, R″₄ and R″₅ are independently selected from the group consisting of: —H, carboxy, —CF₃, halo, methylthio, methylsulfonyl, phenyl, C₁₋₃alkoxy and C₁₋₃alkyl, said C₁₋₃alkyl optionally substituted with carboxy or hydroxy,
R″₆ is H or OH, and
------ is an optional double bond.

3. The compound according to claim 1 wherein D is N.

4. The compound according to claim 1 wherein D is CR₄.

5. The compound according to claim 4 wherein:
R₃ and R₄ are joined together with the carbon atoms to which they are attached to form a six-membered monocyclic ring as follows:

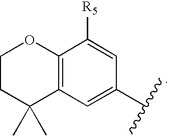

6. The compound according to claim 4 wherein:
R₃ is selected from the group consisting of H, halo, C₁₋₄alkyl and —CF₃;
R₄ is selected from the group consisting of: H, halo, C₁₋₄alkyl, —OCH₃ and —CF₃;
R₅ is selected from the group consisting of: H, halo, C₁₋₄alkyl, CF₃ and —SCF₃; and
R₆ is H.

7. The compound according to claim 6 wherein:
R₃ is selected from the group consisting of: H, Cl, Br, tert-butyl and —CF₃;
R₄ is selected from the group consisting of: H, Cl, Br, tert-butyl, —OCH₃ and —CF₃;
R₅ is selected from the group consisting of: H, Cl, Br, tert-butyl, CF₃ and —SCF₃; and
R₆ is H.

8. The compound according to claim 1 wherein R₆ is —H.

9. The compound according to claim 1 wherein:
D is CR₄;
R₃ is selected from the group consisting of: H, Cl, Br, tert-butyl and —CF₃;
R₄ is selected from the group consisting of: H, Cl, Br, tert-butyl, —OCH₃ and —CF₃;
or R₃ and R₄ may be joined together with the carbon atoms to which they are attached to form a six-membered monocyclic ring as follows: 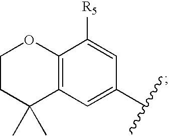
R₅ is selected from the group consisting of H, Cl, Br, tert-butyl, CF₃ and —SCF₃; and
R₆ is —H.

10. The compound according to claim 9 wherein:
(1) R₃ is tert-butyl, R₄ is H and R₅ is tert-butyl;
(2) R₃ is tert-butyl, R₄ is —OCH₃ and R₅ is tert-butyl;
(3) R₃ is —CF₃, R₄ is —H and R₅ is —CF₃;
(4) R₃ is H, R₄ is —OCH3 and R₅ is H;
(5) R₃ is H, R₄ is tert-butyl and R₅ is H;
(6) R₃ is H, R₄ is Cl and R₅ is H;
(7) R₃ is H, R₄ is Br and R₅ is H;
(8) R₃ is H, R₄ is —CF₃ and R₅ is H;
(9) R₃ is H, R₄ is H and R₅ is Cl;
(10) R₃ is H, R₄ is H and R₅ is Cl;
(11) R₃ is H, R₄ is H and R₅ is —CF₃; or
(12) R₃ is Cl, R₄ is H and R₅ is Cl.

11. The compound according to claim 9 wherein 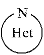
is selected from the group consisting of 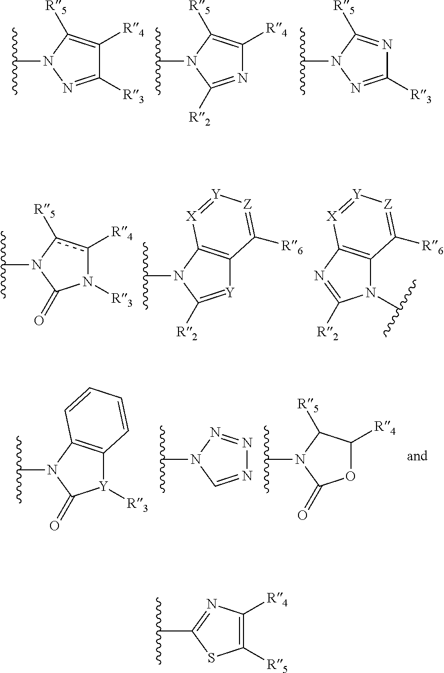
wherein X, Y and Z are independently C or N,
R′₂, R″₃, R″₄ and R″₅ are independently selected from the group consisting of: —H, carboxy, —CF₃, halo, methylthio, methylsulfonyl, phenyl, C₁₋₃alkoxy and C₁₋₃alkyl, said C₁₋₃alkyl optionally substituted with carboxy or hydroxy,
R″₆ is H or OH, and
------ is an optional double bond.

12. The compound according to claim 11 wherein: 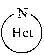
is

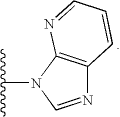

13. The compound according to claim 9 wherein:
D is CR₄;
R₃ is tert-butyl;
R₄ is H or —OCH₃;
R₅ is tert-butyl; and
R₆ is H.

14. A compound according to claim 1 selected from the following group:
(1) 3-(2-{4-[2-(3,5-di-tert-butyl-4-methoxyphenyl)-1,3-thiazol-4-yl}piperidin-1-yl}-2-oxoethyl)-3H-imidazo[4,5-b]pyridine;
(2) 4-[2-(3,5-di-tert-butyl-4-methoxyphenyl)-1,3-thiazol-4-yl]-1-[(2,5-dimethyl-1H-imidazol-1-yl)acetyl]piperidine;
(3) [1-(2-{4-[2-(3,5-di-tert-butyl-4-methoxyphenyl)-1,3-thiazol-4-yl]piperidin-1-yl}-2-oxoethyl)-2-methyl-1H-imidazol-5-yl]methanol;
(4) 1-(2-{4-[2-(3,5-di-tert-butyl-4-methoxyphenyl)-1,3-thiazol-4-yl]piperidin-1-yl}-2-oxoethyl)-3-methyl-1,3-dihydro-2H-imidazol-2-one;
(5) 3-(2-{4-[2-(3,5-di-tert-butyl-4-methoxyphenyl)-1,3-thiazol-4-yl]piperidin-1-yl}-2-oxoethyl)-1,4-dimethyl-1,3-dihydro-2H-imidazol-2-one;
(6) [1-(2-{4-[2-(3,5-di-tert-butyl-4-methoxyphenyl)-1,3-thiazol-4-yl]piperidin-1-yl}-2-oxoethyl)-4-methyl-1H-pyrazol-3-yl]acetate bis(hydrotrifluoroacetate);
(7) 4-[2-(3,5-di-tert-butyl-4-methoxyphenyl)-1,3-thiazol-4-yl]-1-{[4-methyl-2-(methylsulfonyl)-1H-imidazol-1-yl]acetyl}piperidine;
(8) 1-(2-{4-[2-(3,5-di-tert-butyl-4-methoxyphenyl)-1,3-thiazol-4-yl]piperidin-1-yl}-2-oxoethyl)-1,3-dihydro-2H-imidazol-2-one;
(9) Potassium_{—}[3-(2-{4-[2-(3,5-di-tert-butyl-4-methoxyphenyl)-1,3-thiazol-4-yl]piperidin-1-yl}-2-oxoethyl)-2-oxo-2,3-dihydro-1H-imidazol-1-yl]acetate;
(10) 1-(2-{4-[2-(3,5-di-tert-butyl-4-methoxyphenyl)-1,3-thiazol-4-yl]piperidin-1-yl}-2-oxoethyl)-3-phenylimidazolidin-2-one;
(11) 3-{2-[4-(2-{3-tert-butyl-5-[(trifluoromethyl)thio]phenyl}-1,3-thiazol-4-yl)piperidin-1-yl]-2-oxoethyl}-3H-imidazo[4,5-b]pyridine;
(12) 4-[2-(3,5-di-tert-butyl-4-methoxyphenyl)-1,3-thiazol-4-yl]-1H-[(4-methoxy-1-imidazol-1-yl)acetyl]piperidine;
(13) 4-[2-(8-tert-butyl-4,4-dimethyl-3,4-dihydro-2H-chromen-6-yl)-1,3-thiazol-4-yl]-1-[(2,4-dimethyl-1H-imidazol-1-yl)acetyl]piperidine;
(14) 1-[(4-chloro-2,5-dimethyl-1H-imidazol-1-yl)acetyl]-4-[2-(3,5-di-tert-butyl-4-methoxyphenyl)-1,3-thiazol-4-yl]piperidine;
(15) 3-(2-{4-[2-(3,5-di-tert-butylphenyl)-1,3-thiazol-4-yl]piperidin-1-yl}-2-oxoethyl)-3H-imidazo[4,5-c]pyridine;
(16) 1-(2-{4-[2-(3,5-di-tert-butylphenyl)-1,3-thiazol-4-yl]piperidin-1-yl}-2-oxoethyl)-1H-imidazo[4,5-c]pyridine;
(17) 3-(2-{4-[2-(3,5-di-tert-butylphenyl)-1,3-thiazol-4-yl]piperidin-1-yl}-2-oxoethyl)-3H-imidazo[4,5-b]pyridine1;
(18) 1-(2-{4-[2-(3,5-di-tert-butylphenyl)-1,3-thiazol-4-yl]piperidin-1-yl}-2-oxoethyl)-1H-imidazo[4,5-b]pyridine;
(19) 3-(2-{4-[2-(3,5-di-tert-butyl-4-methoxyphenyl)-1,3-thiazol-4-yl]piperidin-1-yl}-2-oxoethyl)-1-ethyl-4-methyl-1,3-dihydro-2H-imidazol-2-one;
(20) 1-(2-{4-[2-(3,5-di-tert-butylphenyl)-1,3-thiazol-4-yl]piperidin-1-yl}-2-oxoethyl)-3-ethyl-4-methyl-1,3-dihydro-2H-imidazol-2-one;
(21) 1-(2-{4-[2-(3,5-di-tert-butyl-4-methoxyphenyl)-1,3-thiazol-4-yl]piperidin-1-yl}-2-oxoethyl)-3-methyl-1,3-dihydro-2H-benzimidazol-2-one;
(22) 4-[2-(3,5-di-tert-butyl-4-methoxyphenyl)-1,3-thiazol-4-yl]-1-[(2-ethyl-4-methyl-1H-imidazol-1-yl)acetyl]piperidine;
(23) 2,6-di-tert-butyl-4-{4-[1-(1H-imidazo[4,5-c]pyridin-1-ylacetyl)piperidin-4-yl]-1,3-thiazol-2-yl}phenol;
(24) 2,6-di-tert-butyl-4-{4-[1-(3H-imidazo[4,5-c]pyridin-3-ylacetyl)piperidin-4-yl]-1,3-thiazol-2-yl}phenol;
(25) 2,6-di-tert-butyl-4-{4-[1-(3H-imidazo[4,5-b]pyridin-3-ylacetyl)piperidin-4-yl]-1,3-thiazol-2-yl}phenol;
(26) 2,6-di-tert-butyl-4-(4-{1-[(3,5-dimethyl-1H-1,2,4-triazol-1-yl)acetyl]piperidin-4-yl}-1,3-thiazol-2-yl)phenol;
(27) [1-(2-{4-[2-(3,5-di-tert-butyl-4-methoxyphenyl)-1,3-thiazol-4-yl]piperidin-1-yl}-2-oxoethyl)-4-methyl-1H-imidazol-5-yl]methanol;
(28) 4-[2-(3,5-di-tert-butyl-4-methoxyphenyl)-1,3-thiazol-4-yl]-1-{2-(methylthio)-1H-imidazol-1-yl]acetyl}piperidine;
(29) 4-[2-(3,5-di-tert-butyl-4-methoxyphenyl)-1,3-thiazol-4-yl]-1-{2-(methylsulfonyl)-1H-imidazol-1-yl]acetyl}piperidine;
(30) [1-(2-{4-[2-(3,5-di-tert-butyl-4-methoxyphenyl)-1,3-thiazol-4-yl]piperidin-1-yl}-2-oxoethyl) 5-methyl-1H-imidazol-4-yl]acetic dihydrochloride;
(31) [1-(2-{4-[2-(3,5-di-tert-butyl-4-methoxyphenyl)-1,3-thiazol-4-yl]piperidin-1-yl)-2-oxoethyl)-4-methyl-1H-imidazol-5-yl]acetic dihydrochloride;
(32) 4-[2-(3,5-di-tert-butyl-4-methoxyphenyl)-1,3-thiazol-4-yl]-1-[(3,5-dimethyl-1H-1,2,4-triazol-1-yl)acetyl]piperidine;
(33) 4-[2-(3,5-di-tert-butylphenyl)-1,3-thiazol-4-yl]-1-[(3,5-dimethyl-1H-1,2,4-triazol-1-yl)acetyl]piperidine;
(34) 4-[2(3,5-di-tert-butyl-4-methoxyphenyl)-1,3-thiazol-4-yl]-1-[(4-methyl-1-imidazol-1-yl)acetyl]piperidine;
(35) 4-[2-(3,5-di-tert-butyl-4-methoxyphenyl)-1,3-thiazol-4-yl]-1-[(5-methyl-1-imidazol-1-yl)acetyl]piperidine;
(36) 1-(2-{4-[2-(3,5-di-tert-butyl-4-methoxyphenyl)-1,3-thiazol-4-yl]piperidin-1-yl}-2-oxoethyl)-3-methylimidazolidin-2-one;
(37) 3-(2-{4-[2-(3,5-di-tert-butyl-4-methoxyphenyl)-1,3-thiazol-4-yl]piperidin-1-yl)-2-oxoethyl)-1,3-oxazolidin-2-one;
(38) 4-[2-(3,5-di-tert-butyl-4-methoxyphenyl)-1,3-thiazol-4-yl]-1-[(3,5-dimethyl-1H-pyrazol-1-yl)acetyl]piperidine;
(39) 4-[2-(3,5-di-tert-butylphenyl)-1,3-thiazol-4-yl]-1-[(3,5-dimethyl-1H-pyrazol-1-yl)acetyl]piperidine;
(40) 1-(2-{4-[2-(3,5-di-tert-butyl-4-methoxyphenyl)-1,3-thiazol-4-yl]piperidin-1-yl}-2-oxoethyl)-1H-pyrrolo[2,3-b]pyridine;
(41) 1-{2-[4-(2-{3-tert-butyl-5-[(trifluoromethyl)thio]phenyl}-1,3-thiazol-4-yl)piperidin-1-yl]-2-oxoethyl}-1H-pyrrolo[2,3-b]pyridine;
(42) 1-(2-{4-[2-(3,5-di-tert-butyl-4-methoxyphenyl)-1,3-thiazol-4-yl]piperidin-1-yl}-2-oxoethyl)-1H-benzimidazole;
(43) 1-(2-{4-[2-(3,5-di-tert-butyl-4-methoxyphenyl)-1,3-thiazol-4-yl]piperidin-1-yl}-2-oxoethyl)-2-methyl-1H-benzimidazole;
(44) 2-[4-(2-{3-tert-butyl-5-[(trifluoromethyl)thio]phenyl}-1,3-thiazol-4-yl)piperidin-1-yl]-2-oxoethyl}-1H-benzimidazole;
(45) 1-{2-[4-(2-{3-tert-butyl-5-[(trifluoromethyl)thio]phenyl}-1,3-thiazol-4-yl)piperidin-1-yl]-2-oxoethyl}-2-methyl-1H-benzimidazole;
(46) 4-[2-(3,5-di-tert-butylphenyl)-1,3-thiazol-4-yl]-1-{[5-methyl-3-(trifluoromethyl)-1H-pyrazol-1-yl]acetyl}piperidine;
(47) 1-{[3,5-bis(trifluoromethyl)-1H-pyrazol-1-yl]acetyl}-4-[2-(3,5-di-tert-butylphenyl)-1,3-thiazol-4-yl]piperidine;
(48) 4-bromo-3,5-dimethyl-1H-pyrazol-1-yl)acetyl]-4-[2-(3,5-di-tert-butylphenyl)-1,3-thiazol-4-yl]piperidine;
(49) 1-[(4-iodo-3,5-dimethyl-1H-pyrazol-1-yl)acetyl]-4-[2-(3,5-di-tert-butylphenyl)-1,3-thiazol-4-yl]piperidine;
(50) 1-(2-{4-[2-(3,5-di-tert-butyl-4-methoxyphenyl)-1,3-thiazol-4-yl]piperidin-1-yl)-2-oxoethyl)imidazolidin-2-one;
(51) [1-(2-{4-[2-(3,5-di-tert-butyl-4-methoxyphenyl)-1,3-thiazol-4-yl]piperidin-1-yl)-2-oxoethyl)-1H-imidazol-2-yl]metanol;
(52) [1-(2-{4-[2-(3,5-di-tert-butyl-4-methoxyphenyl)-1,3-thiazol-4-yl]piperidin-1-yl}-2-oxoethyl)-1H-benzimidazol-2-yl]metanol;
(53) 4-[2-(3,5-di-tert-butyl-4-methoxyphenyl)-1,3-thiazol-4-yl]-1-{[2-(trifluoromethyl)-1H-imidazol-1-yl]acetyl}piperidine;
(54) 4-[2-(3,5-di-tert-butyl-4-methoxyphenyl)-1,3-thiazol-4-yl]-1-{[2-methyl-1H-imidazol-1-yl]acetyl}piperidine;
(55) 4-[2-(3,5-di-tert-butyl-4-methoxyphenyl)-1,3-thiazol-4-yl]-1-(1H-imidazol-1-ylacetyl)piperidine;
(56) [1-(2-{4-[2-(3,5-di-tert-butylphenyl)-1,3-thiazol-4-yl]piperidin-1-yl}-2-oxoethyl)-5-methyl-1H-pyrazol-3-yl]acetic acid;
(57) Potassium (1-{2-[4-(2-{3-tert-butyl-5-[(trifluoromethyl)thio]phenyl}-1,3-thiazol-4-yl)piperidin-1-yl]-2-oxoethyl)-5-methyl-1H-pyrazol-3-yl)acetate;
(58) 4-[2-(3,5-di-tert-butylphenyl)-1,3-thiazol-4-yl]-1-[(2,4-dimethyl-1H-imidazol-1-yl)acetyl]piperidine;
(59) 1-(2-{4-[2-(3,5-di-tert-butylphenyl)-1,3-thiazol-4-yl]piperidin-1-yl}-2-oxoethyl)-3-ethyl-1,3-dihydro-2H-imidazol-2-one;
(60) 3-[2-(4-{2-[3,5-bis(trifluoromethyl)phenyl]-1,3-thiazol-4-yl}piperidin-1-yl)-2-oxoethyl]-3H-imidazo[4,5-b]pyridine;
(61) 4-{2-[3,5-bis(trifluoromethyl)phenyl]-1,3-thiazol-4-yl}-1-[(3,5-dimethyl-1H-1,2,4-triazol-1-yl)acetyl]piperidine;
(62) 3-[2-(4-{2-[3,5-bis(trifluoromethyl)phenyl]-1,3-thiazol-4-yl}piperidin-1-yl)-2-oxoethyl]-3H-imidazo[4,5-c]pyridine;
(63) 1-[2-(4-{2-[3,5-bis(trifluoromethyl)phenyl]-1,3-thiazol-4-yl}piperidin-1-yl)-2-oxoethyl]-1H-imidazo[4,5-c]pyridine;
(64) 1-[2-(4-{2-[3,5-bis(trifluoromethyl)phenyl]-1,3-thiazol-4-yl}piperidin-1-yl)-2-oxoethyl]-2-methyl-1H-imidazo[4,5-c]pyridine;
(65) 1-(2-{4-[2-(3,5-di-tert-butyl-4-methoxyphenyl)-1,3-thiazol-4-yl]piperidin-1-yl}-2-oxoethyl)-1H-imidazole-2-carboxylic acid dihydrochloride;
(66) 1-ethyl-3-{2-oxo-2-{4-(2-phenyl-1,3-thiazol-4-yl)piperidin-1-yl]ethyl}-1,3-dihydro-2H-imidazol-2-one;
(67) 1-ethyl-3-(2-{4-[2-(4-methoxyphenyl)-1,3-thiazol-4-yl]piperidin-1-yl}-2-oxoethyl)-1,3-dihydro-2H-imidazol-2-one;
(68) 1-(2-{4-[2-(4-tert-butylphenyl)-1,3-thiazol-4-yl]piperidin-1-yl}-2-oxoethyl)-3-ethyl-1,3-dihydro-2H-imidazol-2-one;
(69) 1-(2-{4-[2-(4-chlorophenyl)-1,3-thiazol-4-yl]piperidin-1-yl}-2-oxoethyl)-3-ethyl-1,3-dihydro-2H-imidazol-2-one;
(70) 1-(2-{4-[2-(4-bromophenyl)-1,3-thiazol-4-yl]piperidin-1-yl}-2-oxoethyl)-3-ethyl-1,3-dihydro-2H-imidazol-2-one;
(71) 1-(2-{4-[2-(4-trifluoromethylphenyl)-1,3-thiazol-4-yl]piperidin-1-yl}-2-oxoethyl)-3-ethyl-1,3-dihydro-2H-imidazol-2-one;
(72) 1-(2-{4-[2-(3-chlorophenyl)-1,3-thiazol-4-yl]piperidin-1-yl}-2-oxoethyl)-3-ethyl-1,3-dihydro-2H-imidazol-2-one;
(73) 1-(2-{4-[2-(3-bromophenyl)-1,3-thiazol-4-yl]piperidin-1-yl}-2-oxoethyl)-3-ethyl-1,3-dihydro-2H-imidazol-2-one;
(74) 1-(2-{4-[2-(3-trifluoromethylphenyl)-1,3-thiazol-4-yl]piperidin-1-yl}-2-oxoethyl)-3-ethyl-1,3-dihydro-2H-imidazol-2-one;
(75) 1-(2-{4-[2-(3,5-dichlorophenyl)-1,3-thiazol-4-yl]piperidin-1-yl}-2-oxoethyl)-3-ethyl-1,3-dihydro-2H-imidazol-2-one; and
(76) 1-ethyl-3-{2-oxo-2-[4-(2-pyridin-4-yl-1,3-thiazol-4-yl)piperidin-1-yl]ethyl}-1,3-dihydro-2H-imidazol-2-one.

15. A pharmaceutical composition comprising a compound according to claim 1 in combination with a pharmaceutically acceptable carrier.

16. A method for treating a disease or condition mediated by the CXCR3 chemokine receptor comprising administering to a patient in need of such treatment a therapeutically effective amount of a compound according to claim 1.

17. The method according to claim 16 wherein the disease or condition is selected from the group consisting of: acute and chronic transplant rejection, psoriasis, rheumatoid arthritis and multiple sclerosis.
